(19) United States
(12) Patent Application Publication
(10) Pub. No.: US 2004/0176393 A1
(43) Pub. Date: Sep. 9, 2004

(54) Statin-MMP inhibitor combinations

(76) Inventors: Roger Schofield Newton, Ann Arbor, MI (US); Bruce David Roth, Plymouth, MI (US)

Correspondence Address: Heidi M. Berven, Warner-Lambert Company LLC, 2800 Plymouth Road, Ann Arbor, MI, 48105, US

(21) Appl. No.: 10/420,441

(22) Filed: Apr. 22, 2003

Related U.S. Application Data
(63) Continuation of application No. 09/977,162, filed on Oct. 12, 2001, now abandoned.
Application No. 09/977,162 is a continuation of application No. 09/297,592, filed on May 3, 1999, now abandoned.
Application No. 09/297,592 is a 371 of international application No. PCT/US98/24681, filed on Nov. 20, 1998.
(60) Provisional application No. 60/078,265, filed on Mar. 17, 1998.

(51) Int. Cl. A61K 31/495; A61K 31/445; A61K 31/401; A61K 31/366; A61K 31/225

(52) U.S. Cl. 514/255.03; 514/317; 514/423; 514/460; 514/548

(57) ABSTRACT

The invention is a pharmaceutical composition comprising an MMP inhibitor and a statin, said composition being useful for treating vascular diseases.

### CROSS REFERENCE TO RELATED APPLICATIONS

This application U.S. Ser. No. 10/420,441 filed Apr. 22, 2003, is a continuation of U.S. Ser. No. 09/977,162 filed on Oct. 12,2001, which is a continuation of U.S. Ser. No. 09/297,592 filed on May 3, 1999 which claims benefit of priority from PCTI/US/98/24681 filed on Nov. 20, 1998 which is based on United States Provisional Application Serial Number 60/0788,265 filed on Mar. 17, 1998.

### FIELD OF THE INVENTION

This invention concerns a combination of a statin compound, which are known to cause a reduction in plasma levels of low-density lipoproteins (LDL) cholesterol, and an MMP inhibitor, which reduce the breakdown of connective tissues. The combination is useful for treating vascular disorders and preventing heart failure.

### BACKGROUND OF THE INVENTION

Several clinical studies have established that lowering certain forms of cholesterol in a mammal is an effective way to treat and prevent heart attacks, sudden death, and angina, both in subjects having higher than normal levels of circulating cholesterol, as well as those having normal levels of cholesterol. Lowering LDL, the bad form of cholesterol, is now one of the primary objectives of physicians treating patients who have, or who have a high risk of developing, cardiovascular diseases such as coronary heart disease, atherosclerosis, myocardial infarction, stroke, cerebral infarction, and even restenosis following balloon angioplasty. Many physicians are now utilizing cholesterol-lowering agents purely as a prophylactic treatment in healthy subjects whose cholesterol levels are normal, thereby guarding against development of cardiovascular diseases.

The most commonly used cholesterol lowering agents are the statins, which are compounds which inhibit the enzyme 3-hydroxy-3-methylglutaryl-coenzyme A (HMG-CoA) reductase, the enzyme responsible for catalyzing the conversion of HMG-CoA to mevalonate, which is an early and rate-limiting step in the cholesterol biosynthetic pathway.

Compounds which inhibit the enzymes that mediate the breakdown of connective tissues are useful for treating heart failure and associated ventricular dilatation. Such enzymes are known as native matrix metalloproteinases (MMP), which are classes of naturally occurring enzymes found in most mammals. They are zinc proteases that hydrolyze collagens, proteoglycans, and glycoproteins. The classes include gelatinase A and B, stromelysin-1 and -2, fibroblast collagenase, neutrophil collagenase, matrilysin, metalloelastase, and interstitial collagenase. These enzymes are implicated with a number of diseases which result from breakdown of connective tissues, such as rheumatoid arthritis, osteoarthritis, osteoporosis, multiple sclerosis, and even tumor metastasis. We have now discovered that treatment and prevention of vascular diseases can be effected by administering a combination of a statin with an MMP inhibitor.

### SUMMARY OF THE INVENTION

This invention provides a method of treating and preventing heart failure and other vascular diseases in a mammal comprising administering an effective amount of a matrix metalloproteinase inhibitor together with a statin. The invention also provides a method for treating and preventing ventricular dilatation comprising administering an effective amount of a matrix metalloproteinase inhibitor together with a statin.

The methods can be practiced by administering any statin in combination with any MMP inhibitor, e.g., any chemical compound that is effective in inhibiting the biological activity of a matrix metalloproteinase such as collagenase, stromelysin, gelatinase, or elastase. Numerous compounds are known to be matrix metalloproteinase inhibitors, and any of such compounds can be utilized in the method of this invention.

In a preferred embodiment, the matrix metalloproteinase inhibitor to be utilized is a substituted bicyclic compound of the formula

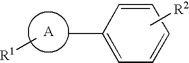

wherein:

A is phenyl or

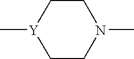

where Y is CH or N;

R¹ is a substituent such as alkyl, aryl, halo, amino, substituted and disubstituted amino, and alkoxy;

R² is carboxyalkyl ketone or oxime, or a carboxyalkyl sulfonamide such as

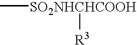

where R³ is alkyl, substituted alkyl, amino, substituted and disubstituted amino, and aryl. Preferred alkyl and alkoxy groups are C₁-C₁₀ alkyl and C₁-C₁₀ alkoxy, which can be straight chain or branched, and optionally substituted by halo, amino, nitro, carboxy, hydroxy, aryl, and heteroaryl.

A particularly preferred embodiment is a method of treating and preventing heart failure and ventricular dilatation by administering a statin together with a biphenylsulfonamide (compounds of the above fonnula when A is phenyl) such as

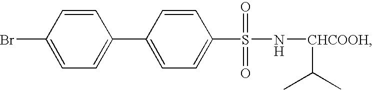

which is also known as CI-1026 and PD 166793.

In another embodiment, CHF and ventricular dilatation is treated or prevented by administering a statin together with a matrix metalloproteinase which is a substituted fused tricyclic compound of the formula

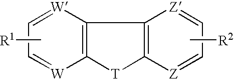

where R¹ and R² are as defined above, T is O, CH₂, SQ (O)_{0,1} or 2, C=O, NR³, or

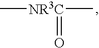

and W, W¹, Z, and Z¹ are each the same or different and each is CR³, where R³ is alkyl, halo, alkoxy, acyl, and aryl. A preferred method utilizes dibenzofurans and fluorenes of the above formula, for instance compounds such as

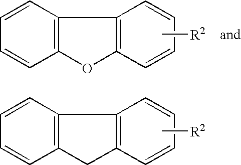

where R² is, for instance,

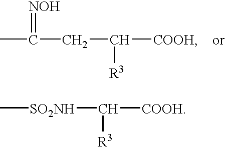

Especially preferred MB inhibitors to be utilized are (S)-2-(dibenzofuran-3-sulfonylamino) -3-methyl-butyric acid and (S)-2-(dibenzofuran-3-sulfonylamino) -succinic acid.

All of the matrix metalloproteinase inhibitors to be utilized in the method of this invention are either known or are readily available by common synthetic processes.

Typical statins to be employed in combination with the MMP inhibitor include atorvastatin, simvastatin, pravastatin, cerivastatin, mevastatin, velostatin, fluvastatin, lovastatin, dalvastatin, and fluindostatin. The statins can be employed as pharmaceutically acceptable salts.

A particularly preferred composition of this invention utilizes a biphenyl-sulfonamide MMP inhibitor together with a statin selected from atorvastatin calcium, pravastatin sodium, simvastatin, lovastatin, and cerivastatin. The most preferred composition employs atorvastatin calcium together with the MMP inhibitor 2-(4-bromobiphenyl4-sulfonylamino)-3-methylbutyric acid.

Also provided by the invention are methods for treating vascular diseases such as peripheral vascular disease, coronary heart disease, stroke, and restenosis.

### DETAILED DESCRIPTION OF THE INVENTION

We have discovered that combining a statin with an MMP inhibitor provides a surprisingly effective composition for treating and preventing vascular diseases. As noted above, the MMP inhibitors and statins are known in the art and are readily available. The compounds can be the free acid, a salt form, or the tetrazolyl or aldehyde analog.

The term “statin”, where used in the specification and the appendant claims, is synonymous with the terms “3-hydroxy-3-methylglutaryl-Coenzyme A reductase inhibitor” and “HMG-CoA reductase inhibitor.” These three terms are used interchangeably throughout the specification and appendant claims. As the synonyms suggest, statins are inhibitors of 3-hydroxy-3-methylglutaryl-Coenzyme A reductase and, as such, are effective in lowering the level of blood plasma cholesterol. Statins and pharmaceutically acceptable salts thereof are particularly useful in lowering low-density lipoprotein cholesterol (LDL-C) levels in mammals and particularly in humans.

The HMG-CoA reductase inhibitors suitable for use herein include, but are not limited to, simvastatin, pravastatin, rivastatin, mevastatin, fluindostatin, cerivastatin, velostatin, fluvastatin, dalvastatin, dihydrocompactin, compactin, or lovastatin; or a pharmaceutically acceptable salt of simvastatin, pravastatin, rivastatin, cerivastatin, mevastatin, fluindostatin, velostatin, fluvastatin, dalvastatin, dihydrocompactin, compactin, lovastatin, or pharmaceutically acceptable salts thereof. However, it is to be noted that atorvastatin calcium is a particularly preferred statin to be employed in the present combination. See U.S. Pat. No. 5,273,995 incorporated herein by reference.

The statins disclosed herein are prepared by methods well-known to those skilled in the art. Specifically, simvastatin may be prepared according to the method disclosed in U.S. Pat. No. 4,444,784, which is incorporated herein by reference. Pravastatin may be prepared according to the method disclosed in U.S. Pat. No. 4,346,227, which is incorporated herein by reference. Cerivastatin may be prepared according to the method disclosed in U.S. Pat. No. 5,502,199, which is incorporated herein by reference. Cerivastatin may alternatively be prepared according to the method disclosed in European Patent Application Publication No. EP617019. Mevastatin may be prepared according to the method disclosed in U.S. Pat. No. 3,983,140, which is incorporated herein by reference. Velostatin may be prepared according to the methods disclosed in U.S. Pat. No. 4,448,784 and U.S. Pat. No. 4,450,171, both of which are incorporated herein by reference. Fluvastatin may be prepared according to the method disclosed in U.S. Pat. No. 4,739,073, which is incorporated herein by reference. Compactin may be prepared according to the method disclosed in U.S. Pat. No. 4,804,770, which is incorporated herein by reference. Lovastatin may be prepared according to the method disclosed in U.S. Pat. No. 4,231,938, which is incorporated herein by reference. Dalvastatin maybe prepared according to the method disclosed in European Patent Application Publication No.738510 A2. Fluindostatin may be prepared according to the method disclosed in European Patent Application Publication No.363934 Al. Dihydrocompactin may be prepared according to the method disclosed in U.S. Pat. No. 4,450,171, which is incorporated herein by reference.

It will be recognized that certain of the above statins contain either a free carboxylic acid or a free amine group as part of the chemical structure. Further, certain statins within the scope of this invention contain lactone moieties, which exist in equilibrium with the free carboxylic acid form. These lactones can be maintained as carboxylates by preparing pharmaceutically acceptable salts of the lactone. Thus, this invention includes pharmaceutically acceptable salts of those carboxylic acids or amine groups. The expression “pharmaceutically acceptable salts” includes both pharmaceutically acceptable acid addition salts and pharmaceutically acceptable cationic salts. The expression “pharmaceutically acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl -1,3-propanediol) and procaine. The expression “pharmaceutically acceptable add addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

The pharmaceutically acceptable cationic salts of statins containing free carboxylic acids may be readily prepared by reacting the free acid fonn of the statin with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine, and tromethamine. The salt is isolated by concentration to dryness or by addition of a non-solvent. In many cases, salts are preferably prepared by mixing a solution of the acid with a solution of a different salt of the cation (sodium or potassium ethylhexanoate, magnesium oleate), employing a solvent (e.g., ethyl acetate) from which the desired cationic salt precipitates, or can be otherwise isolated by concentration and/or addition of a non-solvent.

The pharmaceutically acceptable acid addition salts of statins containing free amine groups may be readily prepared by reacting the free base form of the statin with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate), or the dihydrogen form of a tribasic acid (e.g., the dihydrogen phosphate, the citrate), at least one molar equivalent and usually a molar excess of the acid is employed. However, when such salts as the sulfate, the hemisuccinate, the hydrogen phosphate, or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

In addition, the MMP inhibitors and pharmaceutically acceptable acid addition salts thereof may occur as hydrates or solvates. Further, the statins of the instant invention and the pharmaceutically acceptable salts of the statins of the instant invention may also occur as hydrates or solvates. Said hydrates and solvates are also within the scope of the invention.

A “matrix metalloproteinase inhibitor” as used herein is any chemical compound that inhibits by at least five percent the hydrolytic activity of at least one matrix metalloproteinase enzyme that is naturally occurring in a mammal. Such compounds are also referred to as “MMP inhibitors”. Numerous matrix metalloproteinase inhibitors are known, and all are useful in the method of this invention. For example, 4-biarylbutyric and 5-biarylpentanoic acid derivatives are described in WO 96,15096, which is incorporated herein by reference. The compounds are defined generally as (T)ₓα-B-D-E-G. Over 400 specific compounds are named, and each is incorporated herein and can be employed in this invention. Especially preferred compounds to be utilized include the following:

[1,1′-Biphenyl]-4-butanoic acid, 4′-chloro-α-(2-methylpropyl)-γ-oxo-;

[1,1′-Biphenyl]-4-butanoic acid, 4′-chloro-α-(2-methylpropyl)-γ-oxo-, (S)-;

[1,1′-Biphenyl]-4-butanoic acid, 4′-chloro-α-(2-methylpropyl)-γ-oxo-, (R)-;

[1,1′-Biphenyl]-4-butanoic acid, 4′-chloro-β-(2-methylpropyl)-γ-oxo-, (S);.

[1,1′-Biphenyl]-4-butanoic acid, 4′-chloro-β-(2-methylpropyl)-γ-oxo-, (R)-;

[1,1′-Biphenyl]-4-butanoic acid, 4′-chloro-γ-oxo-;

[1,1′-Biphenyl]-4-butanoic acid, 4′-bromo-γ-oxo-;

[1,1′-Biphenyl]-4-butanoic acid, 4′-fluoro-γ-oxo-;

[1,1′-Biphenyl]-4-butanoic acid, 2′-fluoro-γ-oxo-;

[1,1′-Biphenyl]-4-butanoic acid, 2′-chloro-γ-oxo-;

[1,1′-Biphenyl]-4-butanoic acid, 2′,4′-difluoro-oxo-;

[1,1′-Biphenyl]-4-butanoic acid, 3′-chloro-γ-oxo-;

[1,1′-Biphenyl]-4-butanoic acid, α-(2-methyl-propyl)-γ-oxo-;

[1,1′-Biphenyl]-4-butanoic acid, 4′-bromo-α-(2-methylpropyl)-γ-oxo-;

[1,1′-Biphenyl]-4-butanoic acid, 4′-fluoro-α-(2-methylpropyl)-γ-oxo-;

[1,1′-Biphenyl]-4-butanoic acid, 4′-ethyl-α-(2-methylpropyl)-γ-oxo-;

[1,1′-Biphenyl]-4-butanoic acid, 2′-fluoro-α-(2-methylpropyl)-γ-oxo-;

[1,1′-Biphenyl]-4-butanoic acid, 2′-chloro-α-(2-methylpropyl)-γ-oxo-;

[1,1′-Biphenyl]-4-butanoic acid, 4′-methoxy-α-(2-methylpropyl)-γ-oxo-;

[1,1′-Biphenyl]-4-butanoic acid, 2′4′-difluoro-α-(2-methylpropyl)-γ-oxo-;

[1,1′-Biphenyl]-4-butanoic acid, 4′-methyl-α-(2-methylpropyl)-γ-oxo-;

[1,1′-Biphenyl]-4-butanoic acid, α-(2-methyl-propyl)-γ-oxo4′-pentyl-;

[1,1′-Biphenyl]-4-butanoic acid, 4′-chloro-α-methylene-γ-oxo-;

[1,1′-Biphenyl]-4-butanoic acid, 2′-chloro-α-methylene-γ-oxo-;

[1,1′-Biphenyl]-4-butanoic acid, 4′-chloro-α-methylγ-oxo-;

[1,1′-Biphenyl]-4-butanoic acid, 4′-chloro-γ-oxo-α-pentyl-;

Benzenebutanoic acid, 4-chloro-α-(2-methylpropyl)-γ-oxo-;

Benzenebutanoic acid, 4-methyl-α-methylene-γ-oxo-;

2-Butenoic acid, 4-(4′-chloro[1,1′-biphenyl]-4-yl)-4-oxo-, (E)-;

2-Butenoic acid, 4-[4-(4-chlorophenyoxy)-phenyl]-4-oxo, (E)-;

[1,1′-Biphenyl]-4-butanoic acid, 4′-hydroxy-α-(2-methylpropyl)-γ-oxo-;

[1,1′-Biphenyl]-4-butanoic acid, 4′-chloro-β-methylene-γ-oxo-;

[1,1′-Biphenyl]-4-butanoic acid, 4′-chloro-γ-hydroxy-α-(2-methylpropyl)-;

[1,1′-Biphenyl]-4-butanoic acid, 4′-chloro-γ-hydroxy-α-(2-methylpropyl)-;

2(3H)-Furanone, 5-(4′-chloro[1,1′-biphenyl]-4-yl)dihydro-3-(2-methylpropyl)-;

2(3H)-Furanone, 5-(4′-chloro[1,1′-biphenyl]-4-yl)dihydro-3-(2-methylpropyl)-;

[1,1′-Biphenyl]-4-butanoic acid, 3′,4′-dichloro-γ-oxo-α-(3-phenylpropyl)-;

[1,1′-Biphenyl]-4-butanoic acid, 3′,5′-dichloro-γ-oxo-α-(3-phenylpropyl)-;

[1,1′-Biphenyl]-4-butanoic acid, 4′-(acetyloxy)-γ-oxo-α-(3-phenylpropyl)-;

Benzenepentanoic acid, α-[2-[4-(5-chloro-2-thienyl)phenyl]-2-oxoethyl]-;

2-Furancarboxylic acid, 5-[4-(3-carboxy-1-oxo-6-phenylbexyl)phenyl]-;

Benzenepentanoic acid, α-[2-oxo-2-[4-(3-pyridinyl)phenyl]ethyl]-;

Benzenepentanoic acid, α-[2-oxo-2-[4-[6-(pentyloxy)-3-pyridinyl]phenyl]ethyl]-;

[1,1′-Biphenyl]-4-butanoic acid, γ-oxo-4′-(pentylthio)-α-(3-phenylpropyl)-;

[1,1′-Biphenyl]-4-butanoic acid, 4′-methoxy-γ-oxo-α-(3-phenylpropyl)-;

[1,1′-Biphenyl]-4-butanoic acid, 3′-chloro-4′-fluoro-γ-oxo-α-(3-phenylpropyl)-;

[1,1′-Biphenyl]-4-butanoic acid, 4′-ethoxy-γ-oxo-α-(3-phenylpropyl)-;

Benzenepentanoic acid, α-[2-oxo-2-[4(3-thienyl)phenyl]ethyl]-;

[1,1′-Biphenyl]-4-butanoic acid, 2′,4′-dichloro-γ-oxo-α-(3-phenylpropyl)-;

[1,1′-Biphenyl]-4-butanoic acid, 4′-foormyl-γ-oxo- α-(3-phenylpropyl)-;

[1,1′-Biphenyl]-4-butanoic acid, γ-oxo-α-(3-phenylpropyl)-3′,5′-bis(trifluoromethyl)-;

Benzenepentanoic acid, α-[2-oxo-2-[4-(2-thienyl)phenyl]ethyl]-;

[1,1′-Biphenyl]-4-butanoic acid, γ-oxo-α-(3-phenylpropyl)-3′-(trifluoromethyl)-;

[1,1′-Biphenyl]-4-butanoic acid, 2′-formyl-γ-oxo-α-(3-phenylpropyl)-;

[1,1′-Biphenyl]-4-butanoic acid, 4hydroxy-γ-oxo-α-(3-phenylpropyl)-;

[1,1′-Biphenyl]-4-butanoic acid, γ-oxo-α-(3-phenylpropyl)4′-propoxy-;

[1,1′-Biphenyl]-4-butanoic acid, γ-oxo-4′-(pentyloxy)-α-(3-phenylpropyl)-;

[1,1′-Biphenyl]-4-butanoic acid, γ-oxo-4′-(pentyloxy)-α-(3-phenylpropyl)-, (S)-;

[1,1′-Biphenyl]-4-butanoic acid, γ-oxo-4′-(pentyloxy)-α-(3-phenylpropyl)-, (R)-;

[1,1′-Biphenyl]-4-butanoic acid, 4′-(hexyloxy)-γ-oxo-α-(3-phenylpropyl)-;

[1,1′-Biphenyl]-4-butanoic acid, 4′-butoxy-γ-oxo-α-(3-phenylpropyl)-;

[1,1′-Biphenyl]-4-butanoic acid, γ-oxo-4′-(3-phenylpropoxy)-α-(3-phenylpropyl)-;

[1,1′-Biphenyl]-4-butanoic acid, 4′-(1-methylethoxy)-γ-oxo-α-(3-phenylpropyl)-;

[1,1′-Biphenyl]-4-butanoic acid, 4′-(heptyloxy)-γ-oxo-α-(3-phenylpropyl)-;

[1,1′-Biphenyl]-4-butanoic acid, 4′-(cyclohexyl-methoxy)-γ-oxo-α-(3-phenylpropyl)-;

[1,1′-Biphenyl]-4-butanoic acid, 4′-(2-methyl-propoxy)-γ-oxo-α-(3-phenylpropyl)-;

[1,1′-Biphenyl]-4-butanoic acid, γ-oxo-α-(3-phenylpropyl)-4′-(2-propenyloxy)-;

[1,1′-Biphenyl]-4-butanoic acid, 4′-chloro-α-heptyl-γ-oxo-;

[1,1′-Biphenyl]-4-butanoic acid, 4′-chloro-α-decyl-γ-oxo-;

[1,1′-Biphenyl]-4-butanoic acid, 4′-nitro-γ-oxo-α-(2-phenylethyl)-;

[1,1′-Biphenyl]-4-butanoic acid, 4′-cyano-γ-oxo-α-(2-phenylethyl)-;

[1,1′-Biphenyl]-4-butanoic acid, 4′-chloro-α-[2-(2-iodophenyl)ethyl]-γ-oxo-;

[1,1′-Biphenyl]-4-butanoic acid, 4′-chloro-α-[2-(3-iodophenyl)ethyl]-γ-oxo-;

[1,1′-Biphenyl]-4-butanoic acid, 4′-chloro-α-[2-(4-iodophenyl)ethyl]-γ-oxo-;

[1,1′-Biphenyl]-4-butanoic acid, 4′-chloro-α-[2-(3,5-dimethoxyphenyl)ethyl]-γ-oxo-;

[1,1′-Biphenyl]-4-butanoic acid, 4′-chloro-γ-oxo-α-phenyl-;

[1,1′-Biphenyl]-4-butanoic acid, 4′-chloro-γ-oxo-α-(phenylmethyl)-;

[1,1′-Biphenyl]-4-butanoic acid, 4′-chloro-γ-oxo-α-(2-phenylethyl)-;

[1,1′-Biphenyl]-4-butanoic acid, 4′-chloro-γ-oxo-α-[(trimethylsilyl)methyl]-;

[1,1′-Biphenyl]-4-butanoic acid, 4′-bromo-γ-oxo-α-(3-phenylpropyl)-;

[1,1′-Biphenyl]-4-butanoic acid, -γ-oxo-α-(3-phenylpropyl)-;

[1,1′-Biphenyl]-4-butanoic acid, 4′-amino-γ-oxo-α-(2-phenylethyl);

[1,1′-Biphenyl]-4-butanoic acid, γ-oxo-α-(2-phenylethyl)4′-[[(phenylmethoxy)carbonyl]amino]-;

[1,1′-Biphenyl]-4-butanoic acid, 4′-[[(1,1-dimethylethoxy)carbonyl]amino]-γ-oxo-α-(2-phenylethyl)-;

[1,1′-Biphenyl]-4-butanoic acid, 4′-(acetylamino)-γ-oxo-α-(2-phenylethyl)-;

[1,1′-Biphenyl]-4-butanoic acid, γ-oxo-4′-[(1-oxopentyl)amino]-α-(2-phenylethyl)-;

[1,1′-Biphenyl]-4-butanoic acid, 4′-[(3,3-dimethyl-1-oxobutyl)amino]-γ-oxo-α-(2-phenylethyl)-;

[1,1′-Biphenyl]-4-butanoic acid, 4′-chloro-α-[2-[2-(methoxycarbonyl)phenyl]-ethyl]-γ-oxo;

[1,1′-Biphenyl]-4-butanoic acid, α-[2-(2-carboxyphenyl)ethyl]-4′-chloro-γ-oxo-;

[1,1′-Biphenyl]-4-butanoic acid, 4′-chloro-α-[2-[2-[(diethylamino)carbonyl]phenyl]ethyl]-γ-oxo-;

[1,1′-Biphenyl]-4-butanoic acid, 4′-chloro-α-[2-[3-[(diethylamino)carbonyl]phenyl]ethyl]-γ-oxo-, (S)-;

[1,1′-Biphenyl]4-butanoic acid, 4′-chloro-α-[2-[3-[(diethylamino)carbonyl]phenyl]ethyl]-γ-oxo-, (R)-;

Cyclopentanecarboxylic acid, 2-[(4′-chloro[1,1′-biphenyl]4-yl)carbonyl]-5-[(phenylmethoxy)methyl]-, (1α,2β,5β)-;

Cyclopentanecarboxylic acid, 2-[(4′-chloro[1,1′-biphenyl]4-yl)carbonyl]-5-(phenoxymethyl)-, (1α,2β,5βp)-;

Cyclopentanecarboxylic acid, 2-[(benzoyloxy)- methyl]-5-[(4′-chloro[1,1′-biphenyl]4-yl)carbonyl]-, (1α,2β,5β)-;

1,2-Benzenedicarboxylic acid, 1-[[2-carboxy-3-[(4′-chloro[1,1′-biphenyl]-4-yl) carbonyl]cyclopentyl]- methyl]-2-methyl ester,(1α,2β,3α)-;

Cyclopentanecarboxylic acid, 2-[(4′-chloro[1,1′-biphenyl]4yl)carbonyl]-5-[(2-thienylthio)methyl]-, (1α,2β,5β)-;

Cyclopentanecarboxylic acid, 2-[(benzoylamino)methyl]-5-[(4′-chloro[1,1′-biphenyl]4yl)carbonyl]-, (1α,2β,5β)-;

Cyclopentanecarboxylic acid, 2-[(4′-chloro[1,1′-biphenyl]yl)carbonyl]-5-[[(2-methoxyethoxy)methoxy]methyl]-, (1β,2β,5β)-;

Cyclopentanecarboxylic acid, 2-[(4′-chloro[1,1′-biphenyl]4-yl)carbonyl]-5-[[(phenylmethyl)thio]methyl]-, (1α,2β,5β)-;

Cyclopentanecarboxylic acid, 2-[(4′-chloro[1,1′-biphenyl]yl)carbonyl]-5-[(phenylthio)methyl]-, (1α,2β,5β)-;

Cyclopentanecarboxylic acid, 2-[(4′-chloro[1,1′-biphenyl4yl)carbonyl]-5-[(propylthio)methyl]-, (1α,2β,5β)-;

Cyclopentanecarboxylic acid, 2-[(2-benzothiazolylthio)methyl]-5-[(4′-chloro[1,1′-biphenyl]4yl)carbonyl]-, (1α,2β,5β)-;

Benzoic acid, 2-[[[2-carboxy-3-[(4′-chloro[1,1′-biphenyl]yl)-carbonyl]cyclopentyl]methyl]thio]-, 1-methyl ester, (1α,2β,3α)-;

Cyclopentanecarboxylic acid, 2-[(4′-chloro[1,1′-biphenyl]yl)carbonyl]-5-[[[(phenylmethoxy)carbonyl]-amino]methyl]-, (1α,2β,5β)-;

Benzoic acid, 2-methyl-, [2-carboxy-3-[(4′-chloro[1,1′-biphenyl]-yl)carbonyl]cyclopentyl]methyl ester, (1α,2β,3α)-;

Benzoic acid, 3-methyl-, [2-carboxy-3-[(4′-chloro[1,1′-biphenyl]-4-yl)carbonyl]cyclopentyl]methyl ester, (1α,2β,3α)-;

Benzoic acid, 4-methyl-, [2-carboxy-3-[(4′-chloro[1,1′-biphenyl]-4-yl)carbonyl]cyclopentyl]methyl ester, (1α,2β,3α);

Benzoic acid, 2-methoxy-, [2-carboxy-3-[(4′-chloro[1,1′-biphenyl]-4-yl)carbonyl]cyclopentyl]methyl ester, (1α,2β,3α)-;

Benzoic acid, 3-methoxy-, [2-carboxy-3-[(4′-chloro[1,1′-biphenyl]-4-yl)carbonyl]cyclopentyl]methyl ester, (1α,2β,3α);

Benzoic acid, 4-methoxy-, [2-carboxy-3-[(4′-chloro[1,1′-biphenyl]-4-yl)carbonyl]cyclopentyl]methyl ester, (1α,2β,3α)-;

Cyclopentanecarboxylic acid, 2-[(2-benzoxazolylthio)methyl]-5-[(4′-chloro[1,1′-biphenyl]-4-yl)carbonyl]-, (1α,2β,5β)-;

Cyclopentanecarboxylic acid, 2-[(4′-chloro[1,1′-biphenyl]-4-yl)carbonyl]-5-[(1,3-dihydro4nitro-1,3-dioxo-2H-isoindol-2-yl)methyl]-, (1α,2β,5β)-;

Cyclopentanecarboxylic acid, 2-[(4′-chloro[1,1′-biphenyl]-4-yl)carbonyl]-5-[(1,3-dihydro-5-nitro-1,3-dioxo-2H-isoindol-2-yl)methyl]-, (1α,2β,5β)-;

2H-Benz[f]isoindole-2-butanoic acid, α-[2-(4′-ethoxy[1,1′-biphenyl]-4-yl) -2-oxoethyl]-1,3-dihydro-1,3-dioxo-;

[1,1′-Biphenyl]-4-butanoic acid, α-(acetylamino)-4′-chloro-γ-oxo-;

2H-Isoindole-2-hexanoic acid, α-[2-(4′-chloro[1,1′-biphenyl]4yl)-2-oxoethyl]-1,3-dihydro-1,3-dioxo-;

[1,1′-Biphenyl]-4-butanoic acid, 4′-chloro-α-[[[3-(methoxycarbonyl)phenyl]thio]methyl]-γ-oxo-;

[1,1′-Biphenyl]-4-butanoic acid, 4′-chloro-α-[[(2,6(dimethylphenyl)thio]-methyl]-γ-oxo-;

[1,1′-Biphenyl]-4-butanoic acid, 4′-chloro-α-[[[4fluoro-2-(methoxycarbonyl)phenyl]thio]methyl]-γ-oxo-;

[1,1′-Biphenyl]-4-butanoic acid, 4′-chloro-α-[[[3-[(diethylamino)carbonyl]phenyl]thio]methyl]-γ-oxo-;

[1,1′-Biphenyl]-4-butanoic acid, 4′-chloro-α-[[[2-[(dimethiylamino)carbonyl]phenyl]thio]methyl]-γ-oxo-;

[1,1′-Biphenyl]-4-butanoic acid, 4′-chloro-α-[[[3-[(dimethylamino)carbonyl]phenyl]thio]methyl-]γ-oxo-;

Bicyclo[2.2.1]hept-5-ene-2-carboxylic acid, 3-[[4′-(pentyloxy)[1, 1′-biphenyl]-4-yl]carbonyl]-, (2-endo,3-exo)-;

1-Cyclopentene-1-carboxylic acid, 5-[(4′-chloro[1,1′-biphenyl]-4-yl)-carbonyl]-;

Cyclopentanecarboxylic acid, 2-[(4′-chloro[1,1′-biphenyl]yl)carbonyl]-5-[(phenylmethyl)thio]-, (la,2p,5a)-;

Cyclopentanecarboxylic acid, 2-[(4′-chloro[1,1′-biphenyl4-yl)carbonyl]-5-[(phenylmethyl)thio]-, (la,2p,5p)-;

1-Cyclopentene-1-carboxylic acid, 5-[[4′-(pentyloxy)[1,1′-biphenyl]-4-yl]carbonyl]-;

1-Cyclopentene-1-carboxylic acid, 5-[[4′-(hexyloxy)[1,1′-biphenyl]-4 -yl)lcarbonyl]-;

[1,1′-Biphenyl]-4-butanoic acid, 4′-hydroxy-γ-oxo-α-[(phenylthio)methyl]-;

[1,1′-Biphenyl]-4-butanoic acid, α-[2-[2-[(butylamino)carbonyl]phenyl]ethyl]4′-chloro-γ-oxo-;

[1,1′-Biphenyl]-4- butanoic acid, α-[2-(3-carboxyphenyl)ethyl]-4′-chloro--oxo-;

[1,1′-Biphenyl]-4-butanoic acid, 4′-chloro-α-[2-[3-[(diethylamino)-carbonyl]phenyl]ethyl]-γ-oxo-;

[1,1′-Biphenyl]-4butanoic acid, α-[2-[3-[(butylamino)carbonyl]phenylmethyl]-4′-chloro-γ-oxo-;

[1,1′-Biphenyl]-4-butanoic acid, 4′-chloro-α-[2-[4-[(diethylamino)carbonyl]phenyl]ethyl]-γ-oxo-;

[1,1′-Biphenyl]4butanoic acid, α-[2-[4-[(butylamino)carbonyl]phenyl]ethyl]-4′-chloro-γ-oxo;

[1,1′-Biphenyl]-4-butanoic acid, α-[2-(4-carboxyphenyl)ethyl]4′-chloro-γ-oxo-;

[1,1′-Biphenyl]-4-butanoic acid, 4′-methoxy-γ-oxo-α-(2-phenylethyl)-;

[1,1′-Biphenyl]-4-butanoic acid, 4′-hydroxy-γ-oxo-α-(2-phenylethyl)-;

[1,1′-Biphenyl]-4-butanoic acid, 4′-ethoxy-γ-oxo-α-(2-phenylethyl)-;

[1,1′-Biphenyl]-4-butanoic acid, γ-oxo-α-(2-phenyIethyl)4′-propoxy-;

[1,1′-Biphenyl]-4-butanoic acid, γ-oxo-4′-(pentyloxy)-α-(2-phenylethyl)-;

[1,1′-Biphenyl]-4-butanoic acid, 4′-(hexyloxy)-γ-oxo-α-(2-phenylethyl)-;

[1,1′-Biphenyl]-4-butanoic acid, 4′-butoxy-γ-oxo-α-(2-phenylethyl)-;

[1,1′-Biphenyl]-4-butanoic acid, γ-oxo-α-(2-phenylethyl)-4′-(phenylmethoxy)-;

[1,1′-Biphenyl]-4-butanoic acid, α-[2-(3-iodophenyl)ethyl]-γ-oxo-4′-(pentyloxy)-;

[1,1′-Biphenyl]-4-butanoic acid, α-[2-(3-iodophenyl)ethyl]-γ-oxo-4′-(phenylmethoxy)-;

[1,1′-Biphenyl]-4-butanoic acid, α-[2-(3-[(diethylamino)carbonyl]-phenyl]ethyl]-γ-oxo-4′-(pentyloxy)-;

[1,1′-Biphenyl]-4-butanoic acid, α-[2-(3-[(diethylamino)carbonyl]-phenyl]ethyl]-γ-oxo-4′-(phenylmethoxy)-;

1,2-Pyrrolidinedicarboxylic acid, 3-[(4′-chloro[1,1′-biphenyl]-4-yl)carbonyl]-, 1-(phenylmethyl) ester, (2S-trans)-;

1,2-Pyrrolidinedicarboxylic acid, 3-[(4′-chloro[1,1′-biphenyl]4-yl)carbonyl]-, 1-(phenylmethyl) ester, (2′R-trans)-;

L-Proline, 3-[(4′-chloro[1,1′-biphenyl]4-yl)carbonyl]-1-[[(phenylmethyl)-amino]carbonyl]-, trans-;

L-Proline, 3-[(4′-chloro[1,1′-biphenyl]4-yl)carbonyl]-(1-oxo-3-phenylpropyl)-, trans-;

L-Proline, 3-[(4′-chloro[1,1′-biphenyl]4-yl)carbonyl]-1-(phenylacetyl)-, trans-;

L-Proline, 3-[(4′-chloro[(1,1′-biphenyl]4-yl)carbonyl]-1-(3,3-dimethyl-1-oxobutyl)-, trans-;

[1,1′-Biphenyl]-4-butanoic acid, 4′-chloro-α-heptyl-γ-oxo-;

[1,1′-Biphenyl]-4-butanoic acid, 4′-chloro-α-decyl-γ-oxo-;

[1,1′-Biphenyl]-4-butanoic acid, 4′-nitro-γ-oxo-α-(2-phenylethyl)-;

[1,1′-Biphenyl]-4-butanoic acid, 4′-cyano-γ-oxo-α-(2-phenylethyl)-;

[1,1′-Biphenyl]-4-butanoic acid, 4′-chloro-α-[2-(2-iodophenyl)ethyl]-γ-oxo-;

[1,1′-Biphenyl]-4-butanoic acid, 4′-chloro-α-[2-(3-iodophenyl)ethyl]-γ-oxo;

[1,1′-Biphenyl]-4-butanoic acid, 4′-chloro-α-[2-(4-iodophenyl)ethyl]-γ-oxo-;

[1,1′-Biphenyl]-4-butanoic acid, 4′-chloro-α-[2-(3,5-dimethoxyphenyl)ethyl]-γ-oxo-;

[1,1′-Biphenyl]-4-butanoic acid, 4′-chloro-γ-oxo-α-phenyl-;

[1,1′-Biphenyl]-4-butanoic acid, 4′-chloro-γ-oxo-α-(phenylmethyl)-;

[1,1′-Biphenyl]-4-butanoic acid, 4′-chloro-γ-oxoα-(2-phenylethyl)-;

[1,1′-Biphenyl]-4-butanoic acid, 4′-chloro-γ-oxo-α-[(trimethylsilyl)methyl]-;

[1,1′-Biphenyl]-4-butanoic acid, 4′-bromo-γ-oxoα-(3-phenylpropyl)-;

[1,1′-Biphenyl]-4-butanoic acid, γ-oxo-α-(3-phenylpropyl)-;

[1,1′-Biphenyl]-4-butanoic acid, 4′-amino-γ-oxoα-(2-phenylethyl)-;

[1,1′-Biphenyl]-4-butanoic acid, γ-oxo-α-(2-phenylethyl)-4′-[[(phenylmethoxy)carbonyl]amino]-;

[1,1′-Biphenyl]-4-butanoic acid, 4′-[[(1,1-dimethylethoxy)carbonyl]amino]-γ-oxo-α-(2-phenylethyl)-;

[1,1′-Biphenyl]-4-butanoic acid, 4′-(acetylamino)-γ-oxo-α-(2-phenylethyl)-;

[1,1′-Biphenyl]-4-butanoic acid, γ-oxo-4′-[(1-oxopentyl)amino]-α-(2-phenylethyl)-;

[1,1′-Biphenyl]-4-butanoic acid, 4′-[(3,3-dimethyl-1-oxobutyl)amino]-1-oxo-α-(2-phenylethyl)-;

[1,1′-Biphenyl]-4-butanoic acid, 4′-chloro-α-[2-[2-methoxycarbonyl)-phenyl]ethyl)-γ-oxo;

[1,1′-Biphenyl]butanoic acid, α-[2-(2-carboxyphenyl)ethyl]4chloro- γ-oxo-;

[1,1′-Biphenyl]-4-butanoic acid, 4′-chloro-α-[2-[2-[(diethylamino)carbonyi)phenyl]ethyl]-γ-oxo-;

[1,1′-Biphenyl]4-butanoic acid, 4′-chloro-α-[2-[3-[(diethylamino)carbonyl)phenyl]ethyl]-γ-oxo-, (S)-; and

[1,1′-Biphenyl]4-butanoic acid, 4′-chloro-α-[2-[3-[(diethylaminocarbonyl)phenyl]ethyl]-γ-oxo-, (R)-.

Fenbufen and compounds related to fenbufen can be utilized. Such compounds are described in U.S. Pat. No. 3,784,701 and by Child, et al., J. Pharm. Sci., 1977; 66:466476, and Arzneim-Forsch, 1980; 30(4A): 695-702, all of which are incorporated herein by reference. Preferred compounds from the fenbufen series to be utilized in this invention have the formula

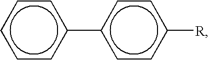

where R is

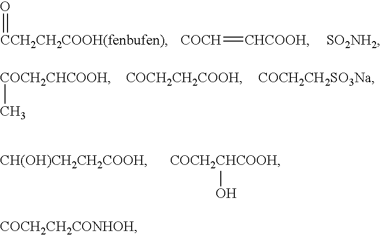

C(=NOH)CH₂CH₂COOH, and COCH₂SCH₂COOH.

Numerous peptides are known matrix metalloproteinase inhibitors. Typical of such peptides are those described in U.S. Pat. No. 5,300,501; 5,530,128; 5,455,258; 5,552,419; WO 95/13289; and WO 96111209, all of which are incorporated herein by reference. Such compounds are illustrated by the formula

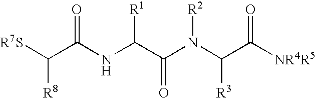

where each of the variable groups can include hydrogen alkyl, aryl, heteroaryl, alkenyl, alkynyl, carboxy, and the like. Preferred compounds from within this class which can be utilized in the method of this invention include the following:

N-[2,3-bis-Acetylmercaptopropanoyl]-L-leucyl-L-phenylalanine N-methylamide;

N-[2-acetylmercapto-3-methoxycarbonylpropanoyl]-L-leucyl-L-phenylalanine N-methylamide;

N-[2-acetylmercapto-4-methoxycarbonylbutanoyl]-L-leucyl-L-phenylalanine N-methylamide;

N-[2-acetylmercapto-5-methoxycarbonylpentanoyl]-L-leucyl-L-phenylalanine N-methylamide;

N-[2-acetylmercapto-6-methoxycarbonylhexanoyl]-L-leucyl-L-phenylalanine N-methylamide;

N-[2-acetylmercapto-4-phthalimidobutanoyl]-L-leucyl-L-phenylalanine N-methylamide;

N-[2-acetylmercapto-5-phthalimidopentanoyl]-L-leucyl-L-phenylaianine N-methylamide;

N-[2-acetylmercapto-6-phthalimidohexanoyl]-L-leucyl-L-phenylalanine N-methylamide;

N-[2,3-bis-mercaptopropanoyl]-L-leucyl-L-phenylalanine N-methylamide;

N-[2-mercapto-3-methoxycarbonylpropanoyl]-L-leucyl-L-phenylalanine N-methylamide;

N-[2-mercapto-4-methoxycarbonylbutanyol]-L-leucyl-L-phenylalanine N-methylamide;

N-[2-mercapto-4-methoxycarbonylpentanoyl]-L-leucyl-L-phenylalanine N-methylamnide;

N-[2-mercapto-6-methoxycarbonylhexanoyl]-L-leucyl-L-phenylalanine N-methylamide;

N-[2-mercapto-4-phthalimidobutanoyl]-L-leucyl-phenyl-alanine N-methylamide;

N-[2-mercapto-5-phthalimidopentanoyl]-L-leucyl-L-phenylalanine N-methylamide;

N-[2-mercapto-6-phthalimidohexanyoyl]-L-leucyl-L-phenylaianine N-methylamide;

N-[2-acetylmercapto-5-methoxycarbonylpentanoyl]-L-leucyl-L-phenylalanine N-methylamide;

N-[2-acetylmercapto-6-methoxycarbonylhexanyol]-L-leucyl-L-phenylalanine N-methylamide;

N-[2-acetylmercapto-6-methoxycarbonylhexanyol]-L-valinyl-L-phenylalanine N-methylamide;

N-[2-acetylmercapto-6-methoxycarbonylhexanyol]-L-leucyl-L-tryptophan N-methylamide;

N-[2-acetylmercapto-5-phthalimidopentanoyl]-L-leucyl-L-phenylalanine N-methylamide;

N-[2-acetylmercapto-5-phthalimidopentanoyl]-L-valinyl-L-phenylalanine N-methylamide;

N-[2-acetylmercapto-5-phthalimidopentanoyl]-L-leucyl-L-tryptophan N-methylamide;

N-[2-acetylmercapto-5-phthalimidopentanoyl]-L-leucyl-L-[β-(4-thiazolyl)]alaine N-methylamide;

N-[2-acetylmercapto-5-phthalimidopentanoyl]-L-leucyl-L-(β-(2-pyridyl)alanine N-methylamide;

N-[2-acetylmercapto-5-phthalimidopentanoyl]-L-leucyl-5-methyl-L-glutamicacid N-methylamide;

N-[2-acetylmercapto-6-phthalimidohexanoyl]-L-leucyl-L-phenylalanine N-methylamide;

N-[2-acetylmercapto-2-(3-phthalimido) phenylacetyl]-L-leucyl-L-phenylalanine N-methylamide;

N-[2-mercapto-5-methoxycarbonylpentanoyl]-L-phenylalanine N-methylamide;

N-[2-mercapto-6-methoxycarbonylhexanyol]-L-leucyl-Lphenylalanine N-methylamide;

N-[2-mercapto-6-methoxycarbonylhexanyol]-L-leucyl-L-trptophan N-methylamide;

N-[2-mercapto-5-phthalimidopentanoyl]-L-leucyl-L-phenylalanine N-methylamide;

N-[2-mercapto-5-phthalimidopentanoyl]-L-leucyl-L-tryptophan N-methylamide;

N-[2-mercapto-5-phthalimidopentanoyl]-L-leucyl-L-[β-(4 -thiazolyl)alanine N-methylamide;

N-[2-mercapto-5-phthalimidopentanoyl]-L-leucyl-L-[β-(2-pyridyl)]alanine N-methylamide;

N-[2-mercapto-5-phthalimidopentanoyl]-L-leucyl-5-methyl-L-glutamic acid N-methylamide;

N-[2-mercapto-6phthalimidohexanoyl]-L-leucyl-L-phenylalanine N-methylamide;

N-[N-mercaptoacetyl)-L-leucyl]-L-phenylalanine N-methylamide;

N-[acetomercaptoacyl)-L-leucyl-L-phenylalanine methylamide;

(RS)-2-(acetylthio)pentanoyl-L-leucyl-L-phenylalanine N-methylamide;

(RS)-2-(acetylthio)propanoyl-L-leucyl-L-phenylalanine N-methylamide;

(RS)-2-(acetylthio)-3-methylbutanoyl-L-leucyl-L-phenylalanine N-methylamide;

(RS)-2-(acetylthio)-2-phenylacetyl-L-leucyl-L-phenylalanine N-methylamide;

(RS)-2-(acetylthio)-3-phenylpropanoyl-L-leucyl-L-phenylalanine N-methylamide;

(RS)-2-(acetylthio)-4-phenylbutanoyi-L-leucyl-L-phenylaianine N-methylamide;

N-(acetylmercaptoacyl)-L-threonyl-L-phenylalanine methylamide;

N-(acetylmercaptoacyl)-L-leucyl-L-tryptophan methylamide;

(RS)-2-mercaptopentanoyl-L-leucyl-L-phenylalanine N-methylamide;

(RS)-2-mercaptopropanoyl-L-leucyl-L-phenylalanine N-methylamide;

(RS)-2-mercapto-3-methylbutanoyl-L-leucyl-L-phenylalanine N-methylamide;

(RS)-2-mercapto-2-phenylacetyl-L-leucyl-L-phenylaianine N-methylamide;

(RS)-2-mercapto-3-phenylpropanoyl-L-leucyl-L-phenylalanine N-methylamide;

(RS)-2-mercapto-4-phenylbutanoyl-L-leucyl-L-phenylalanine N-methylamide;

N-[N-(mercaptoacetyl)L-threonyl]-L-phenylalanine methylamide; and

N-[N-(mercaptoacetyl)-L-leucyl]-L-tryptophan methylamide.

Additional matrix metalloproteinase (MMP) inhibitors, which can be utilized to prevent and treat heart failure and ventricular dilatation, include the following:

[4-(N-Hydroxyamino)-2(R)-cyclohexylmethylsuccinyl]-L-β-cyclohexylalanine -N-(2-phenylethyl)amide;

[4-N-(Hydroxyamino)-2R-isobutylsuccinyl]-L-β-cyclohexylalanine-N-(2-phenylethyl)amide;

[4-(N-hydroxyamino)-2R-phenylpropylsuccinyl]-L-β-cyclohexylalanine-N-(2-phenylethyl)amide;

[4-(N-Hydroxyamino)-2R-phenylpropylsuccinyl]-L-β-cyclohexylalanine-N-(2-(N,N-dimethylamino]ethyl)amide;

[4-(N-Hydroxyamino)-2R-phenylpropylsuccinyl]-L-β-cyclohexylalanine-N-[2-(p-sulphonamidophenyl)ethyl)amide;

[4-(N-Hydroxyamino)-2R-phenylpropylsuccinyl]-L-β-cyclohexylalanine-N -(2-(p-sulphonylphenyl)ethyl)amide;

[4-(N-Hydroxyamino)-2R-phenylpropylsuccinyl]-L-β-cyclohexylalanine-N-[2(2-pyridyl)ethyl]amide;

[4-(N-Hydroxyamino)-2R-pentylsuccinyl]-L-β-cyclohexylalanine-N-(2-phenylethyl)amide;

[4-(N-Hydroxyamino)-2R-isoamylsuccinyl]-L-β-cyclohexylalanine-N-(2-phenylethyl)amide;

[4-(N-Hydroxyamino)-2R-phenylbutylsuccinyl]-L-β-cyclohexylalanine-N-(2-phenylethyl)amide;

[4-(N-Hydroxyamino)-2R-phenylpropylsuccinyl]-L-β-cyclohexylalanine-N-[3-(4morpholinyl)propyl]amide;

[4-(N-Hydroxyamino)-2R-phenylpropylsuccinyl]-L-β-cyclohexylalanine-N-[β-alanine]amide;

[4-(N-Hydroxyamino)-2R-isobutylsuccinyl]-L-β-cyclohexylalanine amide;

[4-(N-Hydroxyamino)-2R-(3-phenylpropyl)succinyl]-L-β-cyclohexylalanine amide;

[4-(N-Hydroxyamino)-2R-(3-phenylbutyl)succinyl]-L-β-cyclohexylalanine amide;

[4-N-(Hydroxyamino)-2R-phenylethylsuccinyl]-L-leucine-N-(2-phenylethyl)amide;

[4-(N-Hydroxyamino)-2R-phenylpropylsuccinyl]-L-leucine-N-(2-phenylethyl)amide;

[4-(N-Hydroxyamino)-2(R)-isobutylsuccinyl]-L-tryptophan amide;

[4-(N-Hydroxyamino)-2(R)isobutylsuccinyl]-L-valine amide;

[3-Phosphono-2R,S-phenylpropyl-1-oxopropyl]-L-β-cyclohexylalanine-N-(2-phenylethyl)amide, dimethylester;

[3-Phosphono-2R-phenylpropyl-1-oxopropyl]-L-β-cyclohexylalanine-N-(2-phenylethyl)amide;

[3-Phosphono-2S-phenylpropyl-1-oxopropyl]-L-β-cyclohexylalanine-β-alanine;

[3-Phosphono-2R-phenylpropyl-l-oxopropyl]-L-β-cyclohexylalanine;

[3-Phosphono-2S-phenylpropyl-1-oxopropyl]-L-γ-cyclohexylalanine-β-alanine, methyl ester;

[3-Phosphono-2R,S-phenylpropyl-1-oxopropyl]-L-β-cyclohexylalanine-N-[4(3-aminopropyl)morpholine]amide, bromine salt;

[3-Phosphono2R,S-(4-methylphenyl)propyl-1-oxopropyl]-L-β-cyclohexylalanine -N-(2-phenylethyl)amide, diethylester;

[3-Phosphono-2R,S-(4-methylphenyl)propyl-1-oxopropyl]-L-β-cyclohexylalanine -N-(2-phenylethyl)amide;

4-t-Butoxy-2(R)-[3-(2-phenoxyethyl)succinyl]-L-β-clohexylaianine-N-(2-phenylethyl)amnide;

4-Hydroxy-2(R)-[3-(2-phenoxyethyl)succinyl]-L-β-cyclohexylalanine-N-(2-phenylethyl)amide;

4-(N-Hydroxyamino-2(R)-[3-(2-phenoxyethyl)succinyl]-L-β-cyclohexylalanine -N-(2-phenylethyl)amide;

{4Hydroxy-2(R)-[3-(4pyridinium)propyllsuccinyl }-L-β-cyclohexylalanine -N-(2-phenylethyl)amide;

{4(N-Hydroxyamino)-2(R)-[3-(4pyridinium)propyl]succinyl )-L-β-cyclohexylalanine -N-(2-phenylethyl)amide;

{4-(N-Hydroxyamino)2(R)-[3-(N-methyl4-pyridinium)propyl]succinyl}-L-β-cyclohexylalanine-N-(2-phenylethyl)amide;

{4-Hydroxy-2-(R)-[3-(4-methylphenyl)propyl]succinyl}-L-β-cyclohexylalanine-N-[(2-morpholine-sulphonylamino)ethyl]amide;

{4-(N-Hydroxyamino)-2-(R)-[3-(4-methylphenyl)propyl]succinyl}-L-β-cyclohexylalanine-N-[(2-morpholinesulphonylamino)ethyl]amide;

{4-(N-Hydroxyamino)-2-(R)-[3-(4chlorophenyl)propyl]succinyl}-L-β-cyclohexylalanine-N-[(2-morpholinesulphonylamino)ethyl]amide;

{4-N-Hydroxyamino)-2-(R)-[3-(4-methylphenyl)propyl]succinyl}-L-β-cyclohexylalanine-N-[(2dimethylsulphonylamino)propyl]amide;

[4-(N-Hydroxyamino)-2(R)-[3-(4-chlorophenyl)propyl]succinyl]-L-[S-(methyl)penicillamine]-N-methylamide;

[4(N-Hydroxyanino)-2(R)-[3-(4-chlorophenyI)propyl]succinyl]-L-[S-(methyl)penicillamine]amide;

[4(N-Hydroxyamino)-2(R)-[3-(4-chlorophenyl)propyl]succinyl]-L-penicillamine]amide;

{4-(N-Hydroxyamino)-2(R)-[3-(4-chlorophenyl)propyl]succinyl}-L-[S-(methyl)penicillamninesulphone]-N-methylamide;

{4-(N-Hydroxyamino)-2(R)-[3-(4chlorophenyl)propyl]succinyl}-L-[S-(methyl)penicillaminesulphoxide]-N-methylamide;

{4-(N-Hydroxyamino)-2(R)-[3-(4chlorophenyl)propyl]succinyl}-L-penicillamine-N-methylamide;

[4(N-Hydroxyamino)-2(R)-3-(2-methylpropyl)succinyl]-L-[S-methyl)penicillamine]-N-methylamide;

N⁴⁻Hydroxy-N¹⁻-(1-(S)-carbamoyl-2,2-dimethylpropyl)-2-(R)4-(chlorophenylpropyl)succinamide;

N⁴⁻Hydroxy-N¹⁻-(1-(S)-carbamoyl-2,2-dimethylpropyl)-2-(R)-(4-methylphenylpropyl)succinamnide;

N⁴⁻Hydroxy-N¹⁻-(1-(S)-carbamoyl-2,2-dimethylpropyl)-2-(R)-(4-methoxyphenylpropyl)succinamide;

N⁴⁻Hydroxy-N¹⁻-(1-(S)-carbamoyl-2,2-dimethylpropyl)-2-(R)-(4-trifluoromethylphenylpropyl)succinamide;

N⁴⁻Hydroxy-N¹⁻-(1-(S)-carbamoyl-2,2-dimethylpropyl)-2-(R)-(4-chloromethylphenylpropyl)succinamide;

N-[N-(Mercaptoacetyl)-L-leucyl]-L-phenylalanine methylamide;

N-(Acetomercaptoacyl)-L-leucyl]-L-phenylalanine methylamide;

(RS)-2-(Acetylthio)pentanoyl-L-leucyl-L-phenylalanine N-methylamide;

(RS)-2-(Acetylthio)propanoyl-L-leucyl-L-phenylalanine N-methylamide;

(RS)-2-(Acetylthio)-3-methylbutanoyl-L-leucyl-L-phenylalanine N-methylamide;

(RS)-2-(Acetylthio)-2-phenylacetyl-L-leucyl-L-phenylalanine N-methylamide;

(RS)-2-(Acetylthio)-3-phenylpropanoyi-L-leucyl-L-phenylaianine N-methylamide;

(RS)-2-(Acetylthio)4phenylbutanoyl-L-leucyl-L-phenylalanine N-methylamide;

N-(Acetylmercaptoacyl)-L-threonyl-L-phenylalanine methylamide;

N-(Acetylmercaptoacyl)-L-leucyl-L-tryptophan methylamide;

(RS)-2-Mercaptopentanoyl-L-leucyl-L-phenylalanine N-methylamide;

(RS)-2-Mercaptopropanoyl-L-leucyl-L-phenylalanine N-methylamide;

(RS)-2-Mercapto-3-methylbutanoyi-L-leucyl-L-phenylalanine N-methylamide;

(RS)-2-Mercapto-2-phenylacetyl-L-leucyl-L-phenylalanine N-methylamide;

(RS)-2-Mercapto3-phenylpropanoyl-L-leucyl-L-phenylalanine N-methylamide;

(RS)-2-Mercapto4-phenylbutanoyl-L-leucyl-L-phenylalanine N-methylamide;

N-[N-(Mercaptoacetyl)-L-threonyl]-L-phenylalanine methylamide;

N-[N-(Mercaptoacetyl)-L-leucyl]-L-tryptophan methylamide;

N-[2,3-bis-Acetylmercaptopropanoyl]-L-leucyl-L-phenylalanine N-methylamide;

N-[2-Acetylmercapto-3-methoxycarbonylpropanoyl]-L-leucyl-L-phenylalanine N-methylamide;

N-[2-Acetylmercapto-4-methoxycarbonylbutanoyl]-L-leucyl-L-phenylalanine N-methylamide;

N-[2-Acetylmercapto-5-methoxycarbonylpentanoyl]-L-leucyl-L-phenylalanine N-methylamide;

N-[2-Acetylmercapto-6-methoxycarbonylhexanoyl]-L-leucyl-L-phenylalanine N-methylamide;

N-[2-Acetylmercapto-4-phthalimidobutanoyl]-L-leucyl-L-phenylaianine N-methylamnide;

N-[2-Acetylmercapto-5-phthalimidopentanoyl]-L-leucyl-L-phenylalanine N-methylamide;

N-[2-Acetylmercapto-6-phthalimidohexanoyl]-L-leucyl-L-phenylalanine N-methylamide;

N-[2,3-bis-Mercaptopropanoyl]-L-leucyl-L-phenylalanine N-methylamide;

N-[2-Mercapto-3-methoxycarbonylpropanoyl]-L-leucyl-L-phenylaianine N-methylamide;

N-[2-Mercapto-4-methoxycarbonylbutanoyl]-L-leucyl-L-phenylalanine N-methylamide;

N-[2-Mercapto-5-methoxycarbonylpentanoyl]-L-leucyl-L-phenylalanine N-methylamide;

N-[2-Mercapto-6-methoxycarbonylhexanoyl]-L-leucyl-L-phenylalanine N-methylamide;

N-[2-Mercapto-4-phthalimidobutanoyl]-Leucyl-L-phenylalanine N-methylamide;

N-[2-Mercapto-5-phthalimidopentanoyl]-L-leucyl-L-phenylalanine N-methylamide;

N-[2-Mercapto-6-phthalimidohexanoyl]-L-leucyl-L-phenylalanine N-methylamide;

N-[2-Acetylmercapto-5-methoxycarbonylpentanoyl]-L-leucyl-L-phenylalanine N-methylamide;

N-[2-Acetylmercapto-6-methoxycarbonylhexanoyl]-L-leucyl-L-phenylalanine N-methylamide;

N-[2-Acetylmercapto-6-methoxycarbonylhexanoyl]-L-valinyl-L-phenylalanine N-methylamide;

N-[2-Acetylmercapto-6-methoxycarbonylhexanoyl]-L-leucyl-L-tryptophan N-methylamide;

N-[2-Acetylmercapto-5-phthalimidopentanoyl]-L-leucyl-L-phenylalanine N-methylamide;

N-[2-Acetylmercapto-5-phthalimidopentanoyl]-L-valinyl-L-phenylalanine N-methylamide;

N-[2-Acetylmercapto-5-phthalimidopentanoyl]-L-leucyl-L-tryptophan N-methylamide;

N-[2-Acetylmercapto-5-phthalimidopentanoyl]-L-leucyl-L-[β-(4-thiazolyl)]alanine N-methylamide;

N-[2-Acetylmercapto-5-phthalimidopentanoyl]-L-leucyl-L-[β-(2-pyridyl)]alanine N-methylamide;

N-[2-Acetylmercapto-5-phthalimidopentanoyl]-L-leucyl-5-methyl-L-glutamnic acid N-methylamide;

N-[2-Acetylmercapto-6-phthalimidohexanoyl]-L-leucyl-L-phenylalanine N-methylamide;

N-[2-Acetylmercapto-2-(3-phthalimido)phenylacetyl]-L-leucyl-L-phenylalanine N-methylamide;

N-[2-Mercapto-5-methoxycarbonylpentanoyl]-L-leucyl-L-phenylalanine N-methylamide;

N-[2-Mercapto-6-methoxycarbonylhexanoyl]-L-leucyl-L-phenylalanine N-methylamide;

N-[2-Mercapto-6-methoxycarbonylhexanoyl]-L-leucyl-L-tryptophan N-methylamide;

N-[2-Mercapto-5-phthalimidopentanoyl]-L-leucyl-L-phenylalanine N-methylamide;

N-[2-Mercapto-5-phthalimidopentanoyl]-L-leucyl-L-tryptophan N-methylamide;

N-[2-Mercapto-5-phthalimidopentanoyl]-L-leucyl-L[β-(4-thiazolyl)alanine N-methylamide;

N-[2-Mercapto-5-phthalimidopentanoyl]-L-leucyl-L[β-(2-pyridyl)]alanine N-methylamide;

N-[2-Mercapto-5-phthalimidopentanoyl]-L-leucyl-5-methyl-L-glutamic acid N-methylamide;

N-[2-Mercapto4phthalimidohexanoyl]-L-leucyl-L-phenylalanine N-methylamide;

N-Hydroxy-2(R)-[[4-methoxybenzenesulfonyl]-(3-picolyl)amino]-3-methylbutanamide;

N-Hydroxy-2(R)-[[4-methoxybenzenesulfonyl]-3-picolyl)amino]-2-cyclohexylacetamide;

N-Hydroxy-2(R)-[[4-methoxybenzenesulfonyl]-(benzyl)amino]4-methylpentanamide;

N-Hydroxy-2(R)-[[4-methoxybenzenesulfonyl]-(benzyl)amino]-6[(N,N-dimethylglycyl)amino]hexanamide hydrochloride;

N-Hydroxy-2(R)-[[4-methoxybenzenesulfonyl]-(3-picolyl)amino]-3-methylbutanamide;

N-Hydroxy-2(R)-[[4-methoxybenzenesulfonyl]-(4picolyl)amino]-2-cyclohexylacetamide;

N-Hydroxy-2(R)-[(4-methoxybenzenesulfonyl]-(4picolyl)amino]-2-(2-tetrahydrofuranyl)acetamide;

N-Hydroxy-2(R)-[[4-methoxybenzenesulfonyl]-(3-picolyl)amino]-3-methylbutanamide;

[4-(N-Hydroxyamino)-2R-isobutyl-3S-methylsuccinyl]-N²⁻-(S)-piperazic acid N-methyl amide;

[4-01-Hydroxyamino)-2R-isobutyl-3S-benzylsuccinyl]-N²⁻-(S)-piperazic acid N-methyl amide;

[4-(N-Hydroxyamino)-2R-isobutyl-3S-methoxyphenylsuccinyl]-N²⁻-(S)-piperazic acid N-methyl amide;

[4-(N-Hydroxyamino)-2R-isobutyl-3S-methoxybenzylsuccinyl]-N²⁻-(S)-piperazic acid N-methyl amide;

[4-(N-Hydroxyamino)-2R-isobutyl-3S-methyl-thiophenylsuccinyl]-N²⁻-(S)-piperazic acid N-methyl amide;

[4-(N-Hydroxyamino)-2R-isobutyl-3S-methyl-thiobenzylsuccinyl]-N²⁻-(S)-piperazic acid N-methyl amide;

[4-(N-Hydroxyamino)-2R-isobutyl-3S-(methylthio-2-thienyl)succinyl]-N²⁻-(S)-piperazic acid N-methyl amide;

[4-(N-Hydroxyamino)-2R-isobutyl-3S-methylacetate]-N²⁻-(S)-piperazic acid N-methyl amide;

[4-(N-Hydroxyamino)-2R-isobutyl-3S-methyl-isopropanoate]-N²⁻-(S)-piperazic acid N-methyl amide;

[4-(N-Hydroxyamino)-2R-isobutyl-3S-methyl-tert-butanoate]-N²⁻-(S)-piperazic acid N-methyl amide;

[4-(N-Hydroxyamino)-2R-isobutyl-3S-methyl-thioacetate]-N²⁻-(S)-piperazic acid N-methyl amide;

[4-(N-Hydroxyamino)-2R-isobutyl-3S-methyl-thioisopropanoate]-N²⁻-(S)-piperazic acid N-methyl amide;

[4-(N-Hydroxyamino)-2R-isobutyl-3S-methyl-(2-pyridyl)]-N²⁻-(S)-piperazic acid N-methyl amide;

[4-(N-Hydroxyamino)-2R-isobutyl-3S-methyl-(3-pyridyl)]-N²⁻-(S)-piperazic acid N-methyl amide;

[4-(N-Hydroxyamino)-2R-isobutyl-3S-methyl-(4-pyridyl)]-N²⁻-(S)-piperazic acid N-methyl amide;

[4-(N-Hydroxyamino)-2R-isobutyl-3S-methyl thio-tert-butanoate]-N²⁻-(S)-piperazic acid N-methyl amide;

[4-(N-Hydroxyamino)-2R-hexyl-3S-methylsuccinyl]-N²⁻-(S)-piperazic acid N-methyl amide;

[4-(N-Hydroxyamino)-2R-hexyl-3S-benzylsuccinyl]-N²⁻-(S)-piperazic acid N-methyl amide;

[4-(N-Hydroxyamino)-2R-hexyl-3S-methoxyphenylsuccinyl]-N²⁻-(S)-piperazic acid N-methyl amide;

[4-(N-Hydroxyamino)-2R-hexyl-3S-methoxybenzylsuccinyl]-N²⁻-(S)-piperazic acid N-methyl amide;

[4-(N-Hydroxyamino)-2R-hexyl-3S-methylthiophenylsuccinyl]-N²⁻-(S)-piperazic acid N-methyl amide;

[4-(N-Hydroxyamino)-2R-hexyl-3S-methylthiobenzylsuccinyl]-N²⁻-(S)-piperazic acid N-methyl amide;

[4-(N-Hydroxyamino)-2R-hexyl-3S-(methylthio-2-thienyl)succinyl]-N²⁻-(S)-piperazic acid N-methyl amide;

[4-(N-Hydroxyamino)-2R-hexyl-3S-benzylsuccinyl]-N²⁻-(S)-piperazic acid N-methyl amide;

[4-(N-Hydroxyamino)-2R-hexyl-3S-methyl acetate]-N²⁻-(S)-piperazic acid N-methyl amide;

[4-(N-Hydroxyamino)-2R-hexyl-3S-methylisopropanoate]-N²⁻-(S)-piperazic acid N-methyl amide;

[4-(N-Hydroxyamino)-2R-hexyl-3S-methyl tert-butanoate]-N²⁻-(S)-piperazic acid N-methyl amide;

[4-(N-Hydroxyamino)-2R-hexyl-3S-methylthioacetate]-N²⁻-(S)-piperazic acid N-methyl amide;

[4-(N-Hydroxyamino)-2R-hexyl-3S-methylthioisopropanoate]-N²⁻-(S)-piperazic acid N-methyl amide;

[4-(N-Hydroxyamino)-2R-hexyl-3S-methylthio-tert-butanoate]-N²⁻-(S)-piperazic acid N-methyl amide;

[4-(N-Hydroxyamino)-2R-hexyl-3S-methyl-(2-pyridyl)]-N²⁻-(S)-piperazic acid N-methyl amide;

[4-(N-Hydroxyamino)-2R-hexyl-3S-methyl-(3-pyridyl)]-N²⁻-(S)piperazic acid N-methyl amide;

[4-(N-Hydroxyamino)-2R-hexyl-3S-methyl-(4pyridyl)]-N²⁻-(S)piperazic acid N-methyl amide;

[4-(N-Hydroxyamino)-2R-ethylphenyl-3S-methylsuccinyl]-N²⁻-(S)-piperazic acid N-methyl amide;

[4-(N-Hydroxyamino)-2R-ethylphenyl-3S-benzylsuccinyl]-N²⁻-(S)-piperazic acid N-methyl amide;

[4-(N-Hydroxyamino)-2R-ethylphenyl-3S-methoxyphenylsuccinyl]-N²⁻-(S)-piperazic acid N-methyl amide;

[4-(N-Hydroxyamino)-2R-ethylphenyl-3S-methoxybenzylsuccinyl]-N²-(S)-piperazic acid N-methyl amide;

[4-(N-Hydroxyamino)-2R-ethylphenyl-3S-methylthiophenylsuccinyl]-N²⁻-(S)-piperazic acid N-methyl amide;

[4-(N-Hydroxyamino)-2R-ethylphenyl-3S-methylthiobenzylsuccinyl]-N²⁻-(S)-piperazic acid N-methyl amide;

[4-(N-Hydroxyamino)-2R-ethylphenyl-3S-(methylthio-2-thienyl)succinyl]-N²⁻-(S)-piperazic acid N-methyl amide;

[4-(N-Hydroxyamino)-2R-ethylphenyl-3S-benzylsuccinyl]-N²⁻-(S)-piperazic acid N-methyl amide;

[4-(N-Hydroxyamino)-2R-ethylphenyl-3S-methyl acetate]-N²⁻-(S)-piperazic acid N-methyl amide;

[4-(N-Hydroxyamino)-2R-ethylphenyl-3S-methylisopropanoate]-N²⁻-(S)-piperazic acid N-methyl amide;

[4-(N-Hydroxyamino)-2R-ethylphenyl-3S-methyl-tert-butanoate]-N²⁻-(S)-piperazic acid N-methyl amide;

[4-(N-Hydroxyamino)-2R-ethylphenyl-3S-methylthioacetate]-N²⁻-(S)-piperazic acid N-methyl amide;

[4-(N-Hydroxyamino)-2R-ethylphenyl-3S-methylthioisopropanoate]-N²⁻-(S)-piperazic acid N-methyl amide;

[4-(N-Hydroxyamino)-2R-ethylphenyl-3S-methylthio-tert-butanoate]-N²⁻-(S)-piperazic acid N-methyl amide;

[4(N-Hydroxyamino)-2R-octyl-3S-methylsuccinyl]-N²⁻-(S)-piperazic acid N-methyl amide;

[4(N-Hydroxyamino)-2R-octyl-3S-methylthiophenylsuccinyl]-N²⁻-(S)-piperazic acid N-methyl amide;

[4(N-Hydroxyamino)-2R-octyl-3S-methylthiobenzylsuccinyl]-N²⁻-(S)-piperazic acid N-methyl amide;

[4(N-Hydroxyamino)-2R-octyl-3S-methylthio-2-thienyl)succinyl]-N¹⁻-(S)-piperazic acid N-methyl amide;

[4(N-Hydroxyamino)-2R-octyl-3S-methyl acetate]-N²-(S)piperazic acid N-methyl amide;

[4-(N-Hydroxyamino)-2R-octyl-3S-methylisopropanoate]-N²⁻-(S)-piperazic acid N-methyl amide;

[4-(N-Hydroxyamino)-2R-octyl-3S-methyl tert-butanoate]-N²⁻-(S)-piperazic acid N-methyl amide;

[4-(N-Hydroxyamino)-2R-octyl-3S-methylthioacetate]-N²⁻-(S)-piperazic acid N-methyl amide;

[4-(N-Hydroxyamino)-2R-octyl-3S-methylthioisopropanoate]-N²⁻-(S)-piperazic acid N-methyl amide;

[4-(N-Hydroxyamino)-2R-octyl-3S-methylthio-tert-butanoate]-N²⁻-(S)-piperazic acid N-methyl amide;

[4-(N-Hydroxyamino)-2R-octyl-3S-methyl-(2-pyridyl)]-N²⁻-(S)-piperazic acid N-methyl amide;

[4-(N-Hydroxyamino)-2R-octyl-3S-methyl-(3-pyridyl)]-N²⁻-(S)-piperazic acid N-methyl amide;

[4-(N-Hydroxyamino)-2R-octyl-3S-methyl-(4pyridyl)]-N²⁻-(S)-piperazic acid N-methyl amide;

[4-(N-Hydroxyamino)-2R-isobutyl-3S-methylsuccinyl]-N²⁻-(S)4′(S/R)-benzylpiperazic acid N-methyl amide;

[4-(N-Hydroxyamino)-2R-isobutyl-3S-methylsuccinyl]-N²-(S)-5′(S/R)-benzylpiperazic acid N-methyl amide;

[4-(N-Hydroxyamino)-2R-isobutyl-3S-methylsuccinyl]-N²-(S)-6′(S/R)-benzylpiperazic acid N-methyl amide;

[4-(N-Hydroxyamino)-2R-isobutyl-3S-methylsuccinyl]-N²-(S)-[5′,6′]benzopiperazic acid N-methyl amide;

N-[1(R)-Carboxy-ethyl]-α-(S)-isobutylglycine-(S)-N²⁻-piperazic acid methyl amide;

N-[1(R)-Carboxy-ethyl]-α-(S)-hexylglycine-(S)-N²⁻-piperazic acid methyl amide;

N-[1(R)-Carboxy-ethyl]-α-(S)-heptylglycine-(S)-N²⁻-piperazic acid methyl amide;

N-[1(R)-Carboxy-ethyl]-α-(S)-octylglycine-(S)-N²⁻-piperazic acid methyl amide;

N-[1(R)-Carboxy-ethyl]-α-(S)-ethylphenylglycine-(S)-N²⁻-piperazic acid methyl amide;

N-[1(R)-Carboxy-ethyl]-α-(S)-propylphenylclycine-(S)-N²⁻-piperazic acid methyl amide;

N-[1(R)-Carboxy-ethylthiobenzyl]-α-(S)-isobutylglycine-(S)-N²⁻-piperazic acid methyl amide;

N-[1(R)-Carboxy-ethylthiobenzyl]-α-(S)-hexylglycine-(S)-N²⁻-piperazic acid methyl amide;

N-[1(R)-Carboxy-ethylthiobenzyl]-α-(S)-ethylphenylglycine-(S)-N²⁻piperazic acid methyl amide;

N-[1(R)-Carboxy-ethylthiobenzyl]-α-(S)-propylphenylglycine-(S)-N²⁻piperazic acid methyl amide;

N-[1(R)-Carboxy-ethyloxybenzyl]-α-(S)-isobutylglycine(S)-N²⁻piperazic acid methyl amide;

N-[1(R)-Carboxy-ethyloxybenzyl]-α-(S)-hexylglycine-(S)-N²⁻-piperazic acid methyl amide;

N-[1(R)-Carboxy-ethyloxybenzyl]-α-(S)-ethylphenylglycine-(S)-N²⁻piperazic acid methyl amide;

N-[1(R)-Carboxy-ethyloxybenzyl]-α-(S)-propylphenylglycine-(S)-N²⁻piperazic acid methyl amide;

N-[1(R)-Carboxy4(p-toluenesulfonyl)butyl]-α-(S)-phenethylglycyl-(S)-N²⁻piperazic acid methyl amide;

N-[1(R)-Carboxyethyl]-α-[2-(4-phenylphenoxy)ethyl]-glycyl-(S)-N2-piperazic acid methyl amide;

2-[2(R)-[2-[1,1′-Biphenyl)yl]ethyl]-4-butyl-4(S)carboxy-1-oxobutyl]-3(S)-methylaminocarbonyl-hexahydropyridazine;

2-[2(R)-[2-[1,1′-Biphenyl)yl]ethyl]-4-methyl-4(S)carboxy-1-oxobutyl]-3(S)-methylaminocarbonyl-hexahydropyridazine;

2-[2(R)-[2-[1,1′-Biphenyl)yl]propyl]-4-butyl-4(S)-carboxy-1-oxobutyl]-3(S)-methylaminocarbonyl-hexahydropyridazine;

2-[2(R)-[2-(4-Propylphenyl)ethyl]-4-butyl-4(S)-carboxy-1-oxobutyl]-3(S)-methylaminocarbonyl-hexahydropyridazine;

2-[2(R)-[2-(4-Butylphenyl)ethyl]-4-butyl-4(S)-carboxy-1-oxobutyl]-3(S)-methylaminocarbonyl-hexahydropyridazine;

2-[2(R)-[2-(4-t-Butylphenylethyl]-4-butyl4(S)-carboxy-1-oxobutyl]-3(S)-methylaminocarbonyl-hexahydropyridazine;

2-[2(R)-[2-[4-(4-Fluorophenyl)phenyl]ethyl]-4-butyl-4(S)carboxy-1-oxobutyl]-3(S)-methylaminocarbonyl-hexahydropyridazine;

2-[2(R)-[2-[4-(4-Fluorophenyl)phenyl]ethyl]-1-methyl-4(S)-carboxy-1-oxobutyl]-3(S)methylaminocarbonyl-hexahydropyridazine;

2-[2(R)-[2-n-Octyl-1-methyl-4(S)-carboxy-1-oxobutyl]-3(S)-methylaminocarbonyl-hexahydropyridazine;

2-[2(R)-[2-[(4-Thiazolyl)phenylethyl]4butyl-4(S)carboxy-l-oxobutyl]-3(S)-methylaminocarbonyl-hexahydropyridazine;

2-[2(R)-[2-[(4-Thiazolyl)phenyl]ethyl]-4-methyl4(S)-carboxy-1-oxobutyl]-3(S)-methylaminocarbonyl-hexahydropyridazine;

2-[2(R)-[2-[(4Thiazolyl)phenyl]ethyl]-4-[3-(phenylsulfonyl)propyl-4(S)-carboxy-1-oxobutyl]-3(S)-methylaminocarbonyl-hexahydropyridazine;

2-[2(R)-[2-[(4-Thiazolyl)phenyl]ethyl]-4-(3-phenylpropyl)-4-(S)carboxy-1-oxobutyl]-3(S)-methylaminocarbonyl-hexahydropyridazine;

2-[2(R)-[2-[(4-Oxazolyl)phenyl]ethyl]-4-butyl-4(S)-carboxy-1-oxobutyl]-3(S)-methylaminocarbonyl-hexahydropyridazine;

2-[2(R)-[2-[(4-Oxazolyl)phenyl]ethyl]-4-methyl-4(S)-carboxy-1-oxobutyl]-3(S)-methylaminocarbonyl-hexahydropyridazine;

2-[2(R)-[2-[(4-Oxazolyl)phenyl]ethyl]-4-[3-(phenylsulfonyl)propyl-4(S)-carboxy-1-oxobutyl]-3(S)-methylaminocarbonyl-hexahydropyridazine;

2-12(R)-[2-[(4-Oxazolyl)phenyl]ethyl]-4-(3-phenylpropyl)-4(S)carboxy-1-oxobutyl]-3(S)-methylaminocarbonyl-hexahydropyridazine;

2-[2(R)-[2-[4-(Dimethylamino)methylphenyl]ethyl]-4-butyl-4(S)carboxy-1-oxobutyl]-3(S)-methylaminocarbonyl-hexahydropyridazine;

2-[2(R)-[2-[4-(Dimethylamino)methylphenyl]ethyl]-4-methyl-4(S)-carboxy-1-oxobutyl]-3(S)-methylamninocarbonyl-hexahydropyridazine;

2-[2(R)-[2-[4-(Dimethylamino)methylphenyl]ethyl]-4-[3-(phenylsulfonyl)propyl -4(S)carboxy-1-oxobutyl]-3(S)-methylaminocarbonyl-hexahydropyridazine;

2-[2(R)-[2-[4-(Dimethylamino)methylphenyl]ethyl]-4-(3-phenylpropyl)-4(S)carboxy-1-oxobutyl]-3(S)-methylaminocarbonyl-hexahydropyridazine;

2-[2(R)-[2-[(4-Imidazolyl)phenyl]ethyl]-4-butyl-4(S)-carboxy-1-oxobutyl]-3(S)-methylamninocarbonyl-hexahydropyridazine;

2-[2(R)-[2-[(4-Imidazolyl)phenyl]ethyl]-4-methyl-4(S)carboxy-1-oxobutyl]-3(S)-methylamninocarbonyl-hexahydropyridazine;

2-[2(R)-[2-[(4-Imidazolyl)phenyl]ethyl]-4-[3-(phenylsulfonyl)propyl-4(S)-carboxy-1-oxobutyl]-3(S)-methylaminocarbonyl-hexahydropyridazine;

2-[2(R)-[2-[(4Imidazolyl)phenyl]ethyl]-4-[3-(phenylpropyl)-4(S)-carboxy-1-oxobutyl]-3(S)-methylaminocarbonyl-hexahydropyridazine;

HS(CH₂)₂(S-D-Leu)-Phe-NHMe;

HS(S)CHMeCH₂₋(S-D-Leu)-Phe-NEMe;

HS(S)CH(PhtNBu)CH₂₋(S-D-Leu)Phe-NEMe;

HS(S)CH(PhtNEt)CH₂₋(S-D-Leu)-Phe-NEMe;

HS(1,2-Cyclopentyl)(S-D-Leu)-Phe-NHMe

Me-S(NH)₂₋(CH₂₋DL-Leu)-Trp-NHBn;

n-Bu-S(NH)₂₋(CH₂₋DL-Leu)-Trp-NHBn;

n-Bu-S(NH)₂₋(CH₂₋DL-TyrOCH₃)-Trp-NHBn;

Me-RS-SO(NH)-(CH₂₋L-Leu)-Phe-Ala-NH₂;

n-Bu-RS-SO(NH)-(CH₂L-Leu)-Phe-Ala -NH₂;

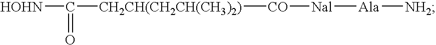

HO-NH-CO-CH₂₋CH-(CH₂₋CH(CH₃)₂₋CO-Nal-Pro-NH₂;

HO-NH-CO-CH(CH₃₋CH(CH₂₋)-CH(CH₃)₂)-CO-Nal-Ala-NH₂;

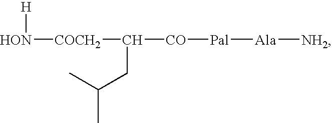

wherein Pal is 3-pyridylalanine;

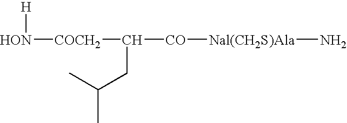

HO-NH-CO-CH₂₋CH(CH₂CH(CH₃)₂)-CONal-(CH₂NH)-Ala-NH₂,

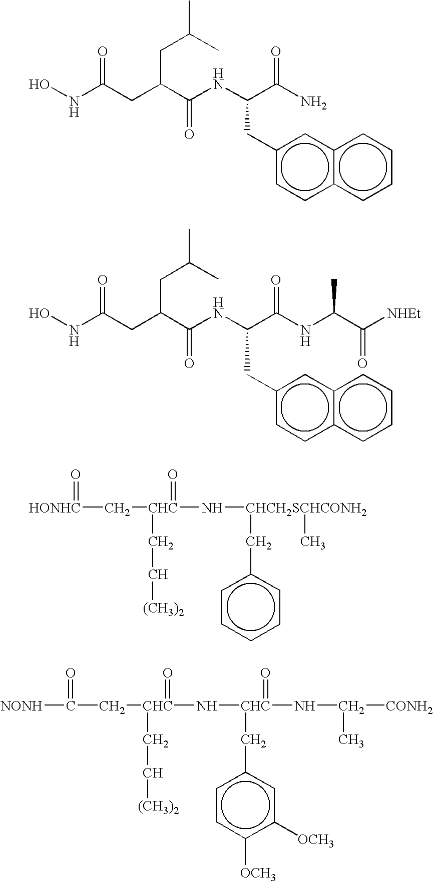

4-(1,3-Dihydro-1,3-dioxo-2H-benz[f]isoindol-2-yl)-2-(R)-[[3-methyl-1-(S) -[[(2-morpholin-4-ylethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[methylamino]carbonyl]butyl]amino]-butanoic acid;

4-(1,3-Dihydro-1,3-dioxo-2H-benz[f]isoindol-2-yl)-2-(R)-[[3-methyl-1-(S) -[[(1H-imidazol-2-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(1H-tetrazol-5-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[[(2-(phenyl)ethyl]amino]carbonyl)butyl]amino]-butanoic acid;

4-(1,3-Dihydro-1,3-dioxo-2H-benz[f]isoindol-2-yl)-2-(R)-[[3-methyl-1-(S) -[[(pyridin-3-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(2-methyl-2H-tetrazo-5-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(4hydroxy-2-methyl-pyrimidin-5-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[2-(2-pyridin-3-yl)ethyl]amino]carbonyl]butyl]amino]-butanoic acid;

4-(1,3-Dihydro-1,3-dioxo-2H-benz[f]isoindol-2-yl)-2-(R)-[[3-methyl-1-(S) -[[l-(1H-tetrazol-5-yl)ethyl]amino]carbonyl]butyl]amino]-butanoic acid;

4-(1,3-Dihydro-1,3-dioxo-2H-benz[f]isoindol-2-yl)-2-(R)-[[3-methyl-1-(S) -[[(5-amino4H-[1,2,4]-triazol-3-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[[1-(6oxo-1,6dihydro-pyridazin-3-yl)ethyl]amino]carbonyl]butyl]amino]-butanoic acid;

4-(1,3-Dihydro-1,3-dioxo-2H-benz[f]isoindol-2-yl)-2-(R)-[[3-methyl-1-(S) -[[(phenyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(benzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(pyridin-4-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[[2-(1H-imidazol4-yl)ethyl]amino]carbonyl]butyl]amino]-butanoic acid;

4-(1,3-Dihydro-1,3-dioxo-2H-benz[f]isoindol-2-yl)-2-(R)-[[3-methyl-1-(S) -[[(pyridin-2-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(4-sulfamoyl-phenyl)amino]carbonyl]butyllamino]-butanoic acid;

4-(1,3-Dihydro-1,3-dioxo-2H-benz[f]isoindol-2-yl)-2-(R)-[[3-methyl-1-(S) -[[(3-sulfamoyl-phenyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(4dimethylamino-benzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[[l-(S)-phenyl-ethyl]amino]carbonyl]butyl]amino]-butanoic acid;

4-(1,3-Dihydro-1,3-dioxo-2H-benz[f]isoindol-2-yl)-2-(R)-[[3-methyl-1-(S) -[[(1,1-dioxo-tetrahydrothiophen-3-yl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(4-sulfamoyl-benzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[[1-(R)-phenyl-ethyl]amino]carbonyl]butyl]amino]-butanoic acid;

4-(1,3-Dihydro-1,3-dioxo-2H-benz[f]isoindol-2-yl)-2-(R)-[[3-methyl-1-(S) -[[(3-fluorobenzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(furan-2-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(1-methyl-1H-tetrazol-5-ylmethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(1,2,3,4tetrahydro-naphthalen-1-yl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(2,4-difluoro-benzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(3-nitrobenzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(4-nitrobenzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(4methanesulfonylamino-benzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(3-methanesulfonylamino-benzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(3,4difluoro-benzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(3-trifluoromethyl-benzyl)amino]carbonyl]butyl]amino]-butanoic acid;

4-[2-(S)-[1-(R)-Carboxy-3-(1,3-dioxo-1,3-dihydro-benzo[f]isoindol-2-yl)-propylamino]4-methyl-pentanoylamino-methyl)-benzoic acid;

4-(1,3-Dihydro-1,3-dioxo-2H-benz[f]isoindol-2-yl)-2-(R)-[[3-methyl-1-(S) -[[(2-hydroxy-1,1-bis-hydroxymethyl-ethyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(3,5-difluoro-benzyl)amino]carbonyl]butyllamino]-butanoic acid;

4-(1,3-Dihydro-1,3-dioxo-2H-benz[f]isoindol-2-yl)-2-(R)-[[3-methyl-1-(S) -[[benzylmethyl-amino]carbonyl]butyl]amino]-butanoic acid;

4-(1,3-Dihydro-1,3-dioxo-2H-benz[f]isoindol-2-yl)-2-(R)-[[3-methyl-1-(S) -[[(2-dimethylaminoethyl)-methyl-amino]carbonyl]butyl]amino]-butanoic acid;

4-(1,3-Dihydro-1,3-dioxo-2H-benz[f]isoindol-2-yl)-2-(R)-[[3-methyl-1-(S) -[[(1-azabicyclo[2.2.2]-oct-3(R)-amino]carbonyl]butyl]amino]-butanoic acid;

4-(1,3-Dihydro-1,3-dioxo-2H-benz[f]isoindol-2-yl)-2-(R)-[[3-methyl-1-(S) -[[(1-azabicyclo[2.2.2]oct-3-(S)-yl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(3-(R)4(S)-5-(R)-6-tetrahydrox-tetrahydra-pyran-2-(R)-ylmethyl)amino]-carbonyl]butyl]amino]-butanoic acid;

4-(1,3-Dihydro-1,3-dioxo-2H-benz[f]isoindol-2-yl)-2-(R)-[[3-methyl-1-(S) -[[(N,N′-dimethyl-hydrazino)carbonyl]butyl]amino]-butanoic acid;

4-(1,3-Dihydro-1,3-dioxo-2H-benz[f]isoindol-2-yl)-2-(R)-[[3-methyl-1-(S) -[[(methylmethoxy)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(dimethyl)amino]carbonyl]butyllamino]-butanoic acid;

4-(1,3-Dihydro-1,3-dioxo-2H-benz[f]isoindol-2-yl)-2-(R)-[[3-methyl-1-(S) -[[(2-oxo-tetrahydro-thiophen-3-(R)-yl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(2-oxo-tetrahydrothiophen-3-(S)-yl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(3-(R)-acetylamino-4-(S)-5-(S)-dihydroxy-6-(R)-hydroxymethyl-tetrahydro-pyran -2-yl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[[benzyl(2-hydroxyethyl)amino]carbonyl]butyl]amino3-butanoic acid;

4-(1,3-Dihydro-1,3-dioxo-2H-benz[f]isoindol-2-yl)-2-(R)-[[3-methyl-1-(S) -[3,4dihydro-1H-isoquinoline-2-carbonyl]butyl]amino]-butanoic acid;

4-(1,3-Dihydro-1,3-dioxo-2H-benz[f]isoindol-2-yl)-2-(R)-[[3-methyl-1-(S) -[4-methylpiperazine-1-carbonyl]butyl]amino]-butanoic acid;

4-(1,3-Dihydro-1,3-dioxo-2H-benz[f]isoindol-2-yl)-2-(R)-[[3-methyl-1-(S) -[1-oxo-[1,4]thiazinane4-carbonyl]butyl]amino]-butanoic acid;

4-(1,3-Dihydro-1,3-dioxo-2H-benz[f]isoindol-2-yl)-2-(R)-[[3-methyl-1-(S) -[morpholine4-carbonyl]butyl]amino]-butanoic acid;

4-(1,3-Dihydro-1,3-dioxo-2H-benz[f]isoindol-2-yl)-2-(R)-[[3-methyl-1-(S) -[4-(2-3hydroxy-propyl)-piperazine-1-carbonyl]butyl]amino]-butanoic acid;

4-(1,3-Dihydro-1,3-dioxo-2H-benz[f]isoindol-2-yl)-2-(R)-[[3-methyl-1-(S) -[3,4,5,6-tetrahydro-H-[2,3]bipyridinyl-1]carbonyl]butyl]amino]-butanoic acid;

4-(1,3-Dihydro-1,3-dioxo-2H-benz[f]isoindol-2-yl)-2-(R)-[[3-methyl-1-(S) -[[(1-methyl-8-oxo-1,7-diazacyclotridec-9-yl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[methyl-1-methyl-piperidin4-yl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(4-hydroxy-1,1-dioxo-tetrahydro-thiophen-3-yl)amino]carbonyl]butyl]-amino]-butanoic acid;

4-(1,3-Dihydro-1,3-dioxo-2H-benz[f]isoindol-2-yl)-2-(R)-[[3-methyl-1-(S) -(4-ethoxycarbonylmethyl-piperazine-1-carbonyl)butyl]amino]-butanoic acid;

4-(1,3-Dihydro-1,3-dioxo-2H-benz[f]isoindol-2-yl)-2-(R)-[[3-methyl-1-(S) -[[(1,1-dioxo-tetrahydro-thiophen-3-yl)-methyl-amino]carbonyl]butyl]amino]-butanoic acid;

4-(1,3-Dihydro-1,3-dioxo-2H-benz[f]isoindol-2-yl)-2-(R)-[[3-methyl-1-(S) -[2-(R)-pyridin-3-yl)-pyrrolidinecarbonyl]butyl]amino]-butanoic acid;

4-(1,3-Dihydro-1,3-dioxo-2H-benz[f]isoindol-2-yl)-2-(R)-[[3-methyl-1-(S) -[2-(S)-(pyridin-3-yl)-pyrrolidinecarbonyl]butyl]amino]-butanoic acid;

4-(1,3-Dihydro-1,3-dioxo-2H-benz[f]isoindol-2-yl)-2-(R)-[[3-methyl-1-(S) -[3-oxo-2-(R)-phenyl-piperazine-1-carbonyl]butyl]amino]-butanoic acid;

4-(1,3-Dihydro-1,3-dioxo-2H-benz[f]isoindol-2-yl)-2-(R)-[[3-methyl-1-(S) -[3-oxo-2-(S)-phenyl-piperazine-1-carbonyl]butyl]amino]-butanoic acid;

4-(1,3-Dihydro-1,3-dioxo-2H-benz[f]isoindol-2-yl)-2-(R)-[[3-methyl-1-(S) -[(pyridine-3-carbonyl-hydrazino)carbonyl]butyl]amino]-butanoic acid;

4-(1,3-Dihydro-1,3-dioxo-2H-benz[f]isoindol-2-yl)-2-(R)-[[3-methyl-1-(S) -[[(benzenesulfonyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[(3-aminobenzyl)amino]carbonyl]butyl]amino]-butanoic acid;

4-(1,3-Dihydro-1,3-dioxo-2H-benz[f]isoindol-2-yl)-2-(R)-[[3-methyl-1-(S) -[[[4-(trifluoro-methanesulfonylamino)benzyl]amino]carbonyl]butyl]amino]-butanoic acid;

4-(1,3-Dihydro-1,3-dioxo-2H-benz[f]isoindol-2-yl)-2-(R)-[[3-methyl-1-(S) -[[[2-hydroxy-(R)-bicyclo[4.3.0]nona-3,6(1)-diene]amino]carbonyl]butyl]-amino]-butanoic acid;

4-(1,3-Dihydro-1,3-dioxo-2H-benz[f]isoindol-2-yl)-2-(R)-[[3-methyl-1-(S) -[[[2-hydroxy-(S)-bicyclo[4.3.0]nona-3,6(1)-diene]amino]carbonyl]butyl]-amino]-butanoic acid;

4-(1,3-Dihydro-1,3-dioxo-2H-benz[f]isoindol-2-yl)-2-(R)-[[3-methyl-1-(S) -[[(N-methyl-pyrrolidine)-methyl-amino]carbonyl]butyl]amino]-butanoic acid;

4-(1,3-Dihydro-1,3-dioxo-2H-benz[f]isoindol-2-yl)-2-(R)-[[3-methyl-1-(S) -[(N-ethoxycarbonylmethyl-piperazine)-1-carbonyl]butyl]amino]-butanoic acid;

2-(R)-[1-(S)-(Benzylamino)carbonyl-3-methylbutylamino]-4-(5-bromo-1,3-dioxo-1,3-dihydro-isoindol-2-yl)-butanoic acid;

2-(R)-[1-(S)-(Benzylamino)carbonyl-3-methylbutylamino]-4-(5-propoxy-1,3-dioxo-1,3-dihydro-isoindol-2-yl)-butanoic acid;

2-(R)-[1-(S)-(Benzylamino)carbonyl-3-methylbutylamino]-4-(5-nitro-1,3-dioxo-1,3-dihydro-isoindol-2-yl)-butanoic acid;

2-(R)-[1-(S)-(Benzylamino)carbonyl-3-methylbutylamino]-4-(5-amino-1,3-dioxo-1,3-dihydro-isoindol-2-yl)-butanoic acid;

2-(R)-[1-(S)-(Benzylamino)carbonyl-3-methylbutylamino]-4-(5-methyl-1,3-dioxo-1,3-dihydro-isoindol-2-yl)-butanoic acid;

2-(R)-[1-(S)-(Benzylamino)carbonyl-3-methylbutylamino]-4-(5-methoxy-1,3-dioxo-1,3-dihydro-isoindol-2-yl)-butanoic acid;

2-(R)-[1-(S)-(Benzylamino)carbonyl-3-methylbutylamino]-4-(5-benzyloxy-1,3-dioxo-1,3-dihydro-isoindol-2-yl)-butanoic acid;

2-(R)-[1-(S)-(Benzylamino)carbonyl-3-methylbutylamino]-4-(5-phenyl-1,3-dioxo-1,3-dihydro-isoindol-2-yl)-butanoic acid;

2-(R)-[1-(S)-(Benzylamino)carbonyl-3-methylbutylamino]-4-(1,3-dioxo-1,3-dihydro-isoindol-2-yl)-butanoic acid;

2-(R)-[1-(S)-(Benzylamino)carbonyl-3-methylbutylamino]-4-(5-methanesulfonylamino1,3-dioxo-1,3dihydro-isoindol-2-yl)-butanoic acid;

2-(R)-[1-(S)-(Benzylamino)carbonyl-3-methylbutylamino]-4-(5-benzenesulfonylamino-1,3-dioxo1,3-dihydro-isoindol-2-yl)-butanoic acid;

2-(R)-[1-(S)-(Benzylamino)carbonyl-3-methylbutylamino]-4-(5-hydroxy-1,3dioxo1,3-dihydroisoindol-2-yl)-butanoic acid;

2-(R)-[[3-Methyl-1-(S)-[[(pyridin-3-ylmethyl)amino]carbonyl]-butyl]amino]4-(1,3,5,7-tetraoxo3,5,6-tetrahydro1H-pyrolo[3,4-f]isoindol-2-yl)butanoic acid;

EtONHCONMe-CH₂CH(iBu)-CO-L-Trp-NHEt;

EtCONOH-CH₂CH(iBu)-CO-L-Trp-NBEt;

n-PrCONOEt-CH₂CH(iBu)-CO-L-Trp-NHEt;

EtNHCONOMe-CH₂CH(iBu)-CO-L-Trp-NHEt;

MeNHCONOH-CH₂CH(iBu)-CO-L-Trp-NHEt;

EtONHCONMe-CH₂CH(iBu)-CO-L-Ala(2-naphthyl)-NHEt;

EtCONOH-CH₂CH(iBu)-CO-L-Ala(2-naphthyl)-NHEt;

n-PrCONOEt-CH₂CH(iBu)-CO-L-Ala(2-naphthyl)-NHEt;

EtNHCONOMe-CH₂CH(iBu)-CO-L-Ala(2-naphthyl)-NHEt;

MeNHCONOH-CH₂CH(iBu)-CO-L-Ala(2-naphthyl)-NHEt;

HONHCONHCH₂CH(iBu)-CO-L-TrpNHMe;

HONHCONHCH₂CH₂CH(iBu)-CO-L-TrpNHMe;

HONHCONHCH(iBu)-CO-L-TrpNHMe;

H₂NCON(OH)CH(iBu)-CO-L-TrpNHMe;

N(OH)CH₂CH(iBu)-CO-L-TrpNEMe;

H₂NCON(OH)CH₂CH₂CH(iBu)-CO-L-TrpNHMe;

CH₃CON(OH)CH(iBu)-CO-L-TrpNHMe;

CH₃CON(OH)CH₂CH(iBu)-CO-L-TrpNHMe;

CH₃CON(OH)CH₂CH₂CH(iBu)-CO-L-TipNHMe;

NHOHCOCH₂CH(i-Bu)CO-L-Trp-NHMe;

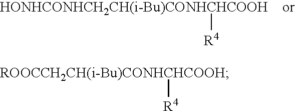

N-{D,L-2-(Hydroxyaminocarbonyl)methyl-4-methylpentanoyl )-L-3-(2′-naphthyl)alanyl-L-alanine, 2-(amino)ethyl amide;

N-{D,L-2-(Hydroxyaminocarbonyl)methyl-4-methylpentanoyl )-L-3-amino-2-dimethylbutanoyl-L-alanine, 2-(amino)ethyl amide;

4(S)-[3-Hydroxyaminocarbonyl-2(R)-(2-methylpropyl)propanoyl]amino-1,2,3,4,5-tetrahydro-3H-2-benzazepin-3-one;

[-4(N-Hydroxyamino)-(2R)-isobutyl-3-methylsuccinyl]-L-phenylglycine-N-methylamide;

4(S)-[2(R)-[l(R)-Hydroxycarbamoyl-2-morpholinoethyl]-4-methylvaleryl]amino-1,2,4,5-tetrahydro-3H-2-benzazepine-3-one;

(1R,4S)-4-[(2R)-Hydroxycarbamoylmethyl-4-methylvaleryl]amino-3-oxo-1,2,4,5-tetrahydro-3H-2-benzazepine-1-carboxylic acid;

3-[2-(N-Methylcarbamoyl)ethylsulfinyl]-5-methylhexanohydroxamic acid;

N-[(2-Thenoylmercapto-3-methyl)-butanoyl]-homocysteine thiolactone;

N-[1(R)-Carboxy-ethyl]-α-(S)-(2-phenyl-ethyl)glycine-(L)-leucine, N-phenylamide;

N-[1(R)-Carboxy-ethyl]-α-(S)-(2-phenyl-ethyl)glycine-(L)-isoleucine, N-phenylamide;

N-[1(R)-Carboxy-ethyl]-α-(S)-(2-phenyl-ethyl)glycine-(L)-alanine, N-phenylamide;

N-[1(R)-Carboxy-ethyl]-α-(S)-(2-pheny1ethyl)glycine-(L)-phenylaianine, N-phenylamide;

N-[1(R)-Carboxy-ethyl]-α-(S)-(2-phenyl-ethyl)glycine-(L)-serine-O-benzyl ether, N-phenylamide;

N-[1(R)-Carboxy-ethyl]-α-(S)-(2-phenylethyl)glycine-(L)-trytophan, N-phenylamide;

N-[1(R)-Carboxy-ethyl]-α-(S)-(2-phenyl-ethyl)glycine-α-(S)-(2-phenyl-ethyl)glycine, N-phenylamide;

N-[1(R)-Carboxy-ethyl]-α-(S)-(2-phenyl-ethyl)glycine-(L)-norleucine, N-phenylamide;

N-[1(R)-Carboxy-ethyl]-α-(S)-(2-phenyl-ethyl)glycine-(L)-valine, N-phenylamide;

N-[1(R)-Carboxy-ethyl]-α-(S)-(2-phenyl-ethyl)glycine-(L)-serine, N-phenylamide hydrochloride;

N-[1(R)-Carboxy-ethyl]-α-(S)-(2-phenyl-ethyl)glycine-(L)-asparagine, N-phenylamide;

N-[1(R)-Carboxy-ethyl]-α-(S)-(2-phenyl-ethyl)glycine-(L)-threonine, N-phenylamide hydrochloride;

N-[1(R)-Carboxy-ethyl]-α-(S)-(2-phenyl-ethyl)glycine-(lysine, N-phenylamide;

N-[1(R)-Carboxy-ethyl]-α-(S)-(2-phenyl-ethyl)glycine-(L)-glutamic acid, N-phenylamide;

N-[1(R)-Carboxy-ethyl]-α-(S)-(2-phenyl-ethyl)glycine-(L)-tyrosine, N-phenylamide hydrochloride;

N-[1(R)-Carboxy-5-(1,3-dioxo-isoindolin-2-yl)pentyl]-α-(S)-(2-phenyl-ethyl)glycine -(L)-leucine, N-phenylamide;

N-[1(R)-Carboxy-5-(1-oxo-isoindolin-2-yl)pentyl]-α-(S)-(2-phenyl-ethyl)-glycine-(S)-leucine, N-phenylamide hydrochloride;

N-[1(R)-Carboxy-5-(1-oxo-isoindolin-2-yl)pentyl,]-α-(S)-(2-phenyl-ethyl)-glycine-(S)-arginine, N-phenylamide;

N-[1(R)-Carboxy-ethyl]-α-(S)-(2-(3-hydroxyphenyl)-ethyl)glycine-(S)-leucine, N-phenylamide hydrochloride;

N-[1(R)-Carboxy-ethyl]-α-(S)-(2-(4-methylphenyl)ethyl)glycine-(S)-leucine, N-phenylamide hydrochloride;

N-[1(R)-Carboxy-ethyl]-α-(S)-(2-(2′-thienyl)ethyl)glycine-(L)-leucine, N-phenylamide;

N-[1(R)-Carboxy-ethyl]-α-(S)-(2-(4ethylphenyl)ethyl)glycine-(L)-leucine, N-phenylamide;

N-[1(R)-Carboxy-5-(1-oxo-isoindolin-2-yl)pentyl]-α-(S)-(2-(4-propylphenyl)ethyl)glycine -(L)-leucine, N-phenylamide;

N-[1(R)-Carboxy-ethyl]-α-(S)-(2-(4chlorophenyl)ethyl)glycine-(L)-leucine, N-phenylamide;

N-[1(R)-Carboxy-ethyl]-α-(S)-(2-phenyl-ethyl)glycine-α-(S)-(2-cyclohexyl-ethyl)glycine, N-phenylamide;

N-[1(R)-Carboxy-ethyl]-α-(S)-(2-phenyl-ethyl)glycine-α-(S)-(cyclohexyl)-glycine, N-phenylamide;

N-[1(R)-Carboxy-ethyl]-α-(S)-(2-phenyl-ethyl)glycine-α-(S)-(cyclohexyl-methyl)glycine, N-phenylamide;

N-[1(R)-Carboxy-ethyl]-α-(S)-(2-phenyl-ethyl)glycine-(L)-β-naphthylalanine, N-phenylamide;

N-[1(R)-Carboxy-ethyl]-α-(S)-(2-phenyl-ethyl)glycine-(L)-α-naphthylalanine, N-phenylamide;

N-[1(R)-Carboxy-ethyl]-α-(S)-(2-phenyl-ethyl)glycine-[(L)-glutamic acid, α,δ-bis-N-phenylamide;

N-[1(R)-Carboxy-ethyl]-α-(S)-(2-phenyl-ethyl)glycine-(L)-leucine, N-cyclohexylamide;

N-[(1(R)-Carboxy-ethyl)]-α-(S)-(2-phenyl-ethyl)glycine-α-(S)-(4-hydroxyphenylethyl)glycine, N-phenylamide;

N-[1(R)-Carboxy-ethyl]-α-(S)-(2-phenyl-ethyl)glycine-(L)-phenylglycine, N-phenylamide;

N-[1(R)-Carboxy-ethyl]-α-(S)-(2-phenyl-ethyl)glycine-(L)-glutamic acid, N_{δ}-benzylamide, N_{α}-phenylamide;

N-[1(R)-Carboxy-ethyl]-α-(S)-(2-phenyl-ethyl)glycine-(L)-ornithine, N-phenylamide;

N-[1(R)-Carboxy-ethyl]-α-(S)-(2-phenyl-ethyl)glycine-(L)-arginine, N-phenylamide;

N-[1(R)-Carboxy-ethyl]-α-(S)-(2-phenyl-ethyl)glycine-α-(S)-(3-phenylpropyl)glycine, N-phenylamide;

N-[1(R)-Carboxy-ethyl]-α-(S)-(2-phenylethyl)glycine-α-(S)-n-octylglycine, N-phenylamide;

N-[1(R)-Carboxy-ethyl]-α-(S)-(2-phenyl-ethyl)glycine-(L)-leucine, N-(4-carboxyphenyl)amide;

N-[1(R)-Carboxy-ethyl]-α-(S)-(2-phenyl-ethyl)glycine-(L)-leucine, N-(4-trifluoromethylphenyl)amide;

N-[1(R)-Carboxy-ethyl]-α-(S)-(2-phenyl-ethyl)glycine-(L)-leucine, N-(3-pyridyl)amide;

N-[1(R)-Carboxy-ethyl]-α-(S)-(2-phenyl-ethyl)glycine-(L)-leucine, N-(benzothiazol-2-yl)amide;

N-[1(R)-Carboxy-ethyl]-α-(S)-(2-(4n-propylphenyl)ethyl)glycine-(L)-leucine, N-phenylamide;

N-[1(R)-Carboxy-ethyl]-α-(S)-(2-4-propylphenylethyl)glycine-(L)-arginine, N-phenylamide;

N-[1(R)-Carboxy-ethyl]-α-(S)-(2-(3,4-dimethylphenyl-ethyl)glycine-(L)-leucine, N-phenylamide;

(2-(((4(1,3-Dihydro-1,3-dioxo-2H-isoindol-2-yl)butyl)hydroxyphosphinyl)methyl)4-phenylbutanoyl)-L-leucine, N-phenylamide;

(2-(((4(1,3-Dihydro-1-oxo-2H-isoindol-2-yl)butyl)hydroxyphosphinyl)-methyl) -4-phenylbutanoyl)-L-leucine, N-phenylamide;

(2-(((4(1,3-Dihydro-1-oxo-2H-isoindol-2-yl)butyl)(2-methyl-1-(1-oxopropoxy) propoxy)phosphinyl)methyl)-4phenylbutanoyl)-L-leucine, N-phenylamide;

(2-((Hydroxy(methyl)phosphinyl)methyl)-4-phenylbutanoyl)-L-leucine, N-phenylamide;

[[Hydroxy[1(R)-[N-(N-acetyl-L-propyl-L-alanyl)-amino]-ethyl]-phosphinyl]-methyl]-4-phenyl-butanoyl-L-leucyl, N-phenylamide;

[Hydroxy-[N-(N-(benzoyl)-L-propyl)aminobutyl]phosphinyl]methyl]-4-phenyl-butanoyl-L-leucine, N-phenylamide;

[Hydroxy-[2-Methylpropyloxycarbonyl-aminobutyl]-phosphinyl]methyl]-4-phenylbutanoyl-L-leucine, N-phenylamide;

[Hydroxy-[1-Methylethylaminocarbonyl-aminobutyl]-phosphinyl]methyl]-4-phenylbutanoyl-L-leucine, N-phenylamide;

N-(2-Thiomethyl-4-phenylbutanoyl)-(L)-leucinamide;

N-(2-Thiomethyl-4-phenylbutanoyl)-(L)-leucine, N-phenylamide;

N-(2-Thiomethyl-4-phenylbutanoyl)-(L)-leucine, N-benzylamide;

N-(2-Thiomethyl-4-phenylbutanoyl)-(L)-leucine, N-(2-phenylethyl)amide;

N-(2-Thiomethyl-4-phenylbutanoyl)-(L)-phenylalaninamide;

N-(2-Thiomethyl-4-phenylbutanoyl)-(L)-phenylalanine N-phenylamide;

N-(2-Thiomethyl-4-phenylbutanoyl)-(L)-phenylalanine N-benzylamide;

N-(2-Thiomethyl-4-phenylbutanoyl)-(L)-phenylalanine-b-alanine;

2(R)-(2-(4-(1-n-Propyl)phenyl)ethyl)-1,5-pentanedioic acid 1-(L-leucine, N-phenylamide)amide;

2(R)-(2-(4-(1-n-Propyl)phenyl)ethyl)-1,5-pentanedioic acid 1-(2(S)-t-butyl)glycine, N-phenylamide)amide;

2(R)-(2-(4-(1-n-Propyl)phenyl)ethyl)-1,5-pentanedioic acid 1-(2(S)-t-butyl)glycine, N-(4-pyridylamide)amide;

2(R)-(2-(4-(1-n-Propyl)phenyl)ethyl)-1,5-pentanedioic acid 1-(L-arginine, N-methylamide)amide;

2(R)-(2-(4-(1-n-Propyl)phenyl)ethyl)-4-methyl-1,5-pentanedioic acid 1-(L-leucine, N-phenylamide)amide;

2(R)-(2-(4-(1-n-Propyl)phenyl)ethyl)-4-methyl-1,5-pentanedioic acid 1-(2(S)-t-butyl)glycine, N-phenylamide)amide;

2(R)-(2-(4(-1-n-Propyl)phenyl)ethyl)-4-methyl-1,5-pentanedioic acid 1-(2(S)-(4-thiazolylmethyl)glycine, N-phenylamide)amide;

2(R)-(2-(4-(1-n-Propyl)phenyl)ethyl)-4-methyl-1,5-pentanedioic acid 1-2(S)-(3-pyridylmethyl)glycine, N-phenylamide)amide;

2(R)-(2-(4-(1-n-Propyl)phenyl)ethyl)-4-methyl-1,5-pentanedioic acid 1-(L-leucine, N-(4-pyridyl)amide)amide;

2(R)-(2-(4-(1-n-Propyl)phenyl)ethyl)-4-methyl-1,5-pentanedioic acid 1-(2(S)-(2-pyridylmethyl)glycine, N-phenylamide)amide;

2(R)-(2-(4(1-n-Propyl)phenyl)ethyl)4methyl-1,5-pentanedioic acid 1-(L-arginine, N-phenylamide)amide;

2(R)-(2-(4-(1-n-Propyl)phenyl)ethyl)-4-methyl-1,5-pentanedioic acid 1-(L-phenylalanine, N-4-pyridylamide)amide;

2(R)-(2-(4-(1-n-Propyl)phenyl)ethyl)-4-(1-(4-(N-(2-oxoisoindolinyl))-butyl)) -1,5-pentanedioic acid 1-(L-leucine, N-phenylamide)amide;

2(R)-(2-(4-(1-n-Propyl)phenyl)ethyl)-4-(1-(4-(N-(2-oxoisoindolinyl))-but-2-enyl)) -1,5-pentanedioic acid 1-(L-leucine, N-phenylamide)amide;

2(R)-(2-(4-(4-Fluorophenyl)phenyl)ethyl)-4methyl-1,5-pentanedioic acid 1-(L-leucine, N-phenylamide)amide;

2(R)-(2-(4-(Phenyl)phenyl)ethyl)-4-methyl-1,5-pentanedioic acid 1-(L-leucine, N-phenylamide)amide;

2(R)-(2-(4-(4-Methoxyphenyl)phenyl)ethyl)-4-methyl-1,5-pentanedioic acid 1-(L-leucine, phenylamide)amide;

2(R)-(2-(4-(4-Methylphenyl)phenyl)ethyl)-4-methyl-1,5-pentanedioic acid 1-(L-leucine, phenylamide)amide;

2(R)-(2-(4-(4Hydroxy-n-butyl)-phenyl)-ethyl)-4-methylpentanedioic acid 1-(S-leucine, phenylamide)amide;

2(R),4(S)-(2-(4(3-Hydroxy-n-propyl)phenyl)ethyl)-4methyl-1,5-pentanedioic acid 1-(L-leucine, N-phenylamide)amide;

2(R)-(2-Phenylethyl)-4-methyl-1,5-pentanedioic acid 1-(L-leucine, N-phenylamide)amide;

2(R)-(2-(4-(1-n-Propyl)phenyl)ethyl)-1,5-pentanedioic acid 1-(L-leucine, N-ethylamide)amide;

2(R)-(2-(4-(1-n-Propyl)phenyl)ethyl)-1,5-pentanedioic acid 1-(L-leucine, N-isopropylamide)amide;

2(R)-(2-(4-(1-n-Propyl)phenyl)propyl)-1,5-pentanedioic acid 1-(2(S)-tert-butyl-glycine, N-pyridyl)amide)amide;

2(R)-(3-(4-(1-n-Propyl)phenyl)propyl)-1,3-pentanedioic acid 1-(L-leucine, N-phenylamide)amide;

2(R)-(2-(4-(1-n-Propyl)phenyl)ethyl)-4-hexyl-1,5-pentanedioic acid 1-(L-leucine, N-phenylamide)amide;

2(R)-(2-(4-(1-n-Propyl)phenyl)ethyl)-4-butyl-1,5-pentanedioic acid 1-(L-leucine, N-phenylamide)amide;

2(R)-(2-(4-(1-n-Propyl)phenyl)ethyl)-4-(3-methylbenzyl)-1,5-pentanedioic acid 1-(L-leucine, N-phenylamide)amide;

2(R)-(2-(4-(1-n-Propyl)phenyl)ethyl)-4-(4(2-benzimidazolyl)butyl)-1,5-pentanedioic acid 1-(L-leucine, N-phenylamide)amide;

2(R) -(2-(4(1-n-Propyl)phenyl)ethyl)-4-(4-(2-benzthiazolyl)butyl)-1,5-pentanedioic acid 1L-leucine, N-phenylamide)amide;

2(R)-(2-(4-(1-n-Propyl)phenyl)ethyl)4(4(2-benzoxazolyl)butyl)-1,5-pentanedioic acid 1-(L-leucine, N-phenylamide)amide;

2(R)-(2-(4-(1-n-Propyl)phenyl)ethyl)4carboxy-1,9-nonanedioic acid 1-(L-leucine, N-phenylamide)amide 1,9-piperidineamide;

2(R)-(2-(4(1-Propyl)phenyl)ethyl)4-carboxy-1,9-nonanedioic acid 1-(L-leucine, N-methylamide)amide 1,9-phenylamide;

2(R-(2-(4(1-n-Propyl)phenyl)ethyl)4carboxy-1,9-nonanedioic acid 1-(L-leucine, N-methylamide)amide 1,9-tert-butylamide;

2(R)-(2-(4(1-n-Propyl)phenyl)ethyl)4carboxy-1,9-nonanedioic acid 1-(L-leucine, N-methylamide)amide 1,9-benzylamide;

2(R)-(2-(4(1-n-Propyl)phenyl)ethyl)4carboxy-1,9-nonanedioic acid 1-(L-leucine, N-methylamide)amide 1,9-morpholineamide;

2(R)-(2-(4(1-n-Propyl)phenyl)ethyl)4carboxy-1,9-nonanedioic acid 1-(L-leucine, N-methylamide)amide 9-(l(R)-phenylethyl)amide;

2(R)-(L-(2-(4-(1-n-Propyl)phenylethyl)4carboxy-1,9-nonanedioic acid 1-(Lleucine, N-methylamide)amide 1,9-(l(S)-phenylethyl)amide;

2(R-(2-(4-(1-n-Propyl)phenyl)ethyl)4carboxy-1,9-nonanedioic acid 1-(Lleucine, N-methylamide)amide 9-(N-methyl-N-phenyl)amide;

2(L)-(2-(4(l-n-Propyl)phenyl)ethyl)4-carboxy-1,9-nonanedioic acid 1-(LIleucine, N-methylamide)amide 9-(N′-methylpiperazine)amide trifluoroacetic acid salt;

2(R)-(2-(4-(1-n-Propyl)phenyl)ethyyl)carboxy-1,9-nonanedioic acid 1-(Lleucine, N-methylamide)amide 9-(3-pyridyl)amide;

2(t)-(2-(4-(1-Propyl)phenyl)ethyl)4carboxy-1,9-nonanedioic acid l-(L-leucine, N-methylamide)amide;

2UR -(2-(4-(1-Propyl)phenyl)ethyl)-1,5-pentanedioic acid 1-((-(S-p-methoxybenzyl)penicillamine, N-phenylamide)amide;

2(R)-(2-(4-(1-Propyl)phenyl)ethyl)-1,5-pentanedioic acid I-((R-(S-p-methoxybenzyl)penicillamine sulfone, N-phenylamide)amide;

2-(2-(4-(1-Propyl)phenyl)ethyl)-4-(1-(4(2-phthalimido))butyl)-1,5- pentandioic acid l-(L-leucine, N-methylamide)amide;

2(L)-(2-(4-(1-n-Propyl)phenyl)ethyl)4(4-benzoylamino-1-butyl)-1,5-pentandioic acid 1-(L leucine, N-methylamide)amide;

2(L)-(2-(4-(1-n-Propyl)phenyl)ethyl)A(4pivaloylamino-1-butyl)-1,5-pentandioic acid I-(L-leucine, N-methylamide)amide;

2(g-(2-(4-(1-n-Propyl)phenyl)ethyl)4-(4-phenylsulfonylamino-1-butyl)-1,5-pentandioic acid 1-(L-leucine, N-methylamide)amide;

2(L-(2- (4-(1-n-Propyl)phenyl)ethyl)4(4 ′-phenylureido)-1-butyl)-1,5-pentandioic acid I-(L-leucine, N-methylamide)amide;

2(R)-(2-(4-(1-n-Propyl)phenyl)ethyl)4(4phenyloxycarbonylamino-1-butyl)-1,5-pentandioic acid 1-(L-leucine, N-methylamide)amide;

2(R)-(2-(4-(1-n-Propyl)phenyl)ethyl)4-(4-N′-benzyloxycarbonylamino-L -propylamino)-1-butyl)-1,5-pentandioic acid 1-(L-leucine, N-methylamide)amide;

2(R)-(2-(4-(1-n-Propyl)phenyl)ethyl)-(4-(4-cyclopentylamino-1-butyl)-1,5-pentandioic acid 1-(L-leucine, N-methylamide)amide;

2(R)-(2-(4(1-n-Propyl)phenyl)ethyl)4(4-(2-carboxybenzoylanino)-1-butyl)-1,5-pentandioic acid 1-(L-leucine, N-methylamide)amide;

2(R)-(2-(4-(1-n-Propyl)phenyl)ethyl)-4(4 -cyano -1-butyl)-1,5-pentandioic acid 1-(L-leucine, N-phenylamide)amide;

N-[1(R)-Carboxyethyl]-α-(S)-(9-amino-nonyl)]glycine-(L)-leucine, N-phenylamide;

N-[1(R)-Carboxyethyl]-α-(S)-(n-octyl)]glycine-(L)-leucine, N-phenylamide;

N-[1(R)-Carboxyethyl]-α-(S)-(n-octyl)]glycine-(L)-arginine, N-phenylamide;

N-[I(R)-Carboxyethyl]-α-(S)-(9-amino-n-nonyl)]glycine-(L)-arginine, N-phenylamide;

N-[1(R)-Carboxyethyl]-α-(S)-(n-decyl)]glycine-(L)-leucine, N-phenylamide;

1-(2-(4-Propylphenyl1thyl)cyclopentane-1,3-dicarboxylic acid 1-(L-leucine, N-phenylamide)amide;

1-(2-(4-Propylphenyl1ethyl)cyclohexane-1,3-dicarboxylic acid 1-(L-leucine, N-phenylamide)amide;

N-[1(R)-Carboxyethyl]-α-(S)-2-(4-fluorobiphenyl)-glycyl-(S)-2-(tert- butyl)glycine, N-phenylamide;

3S-[4(N-Hydroxyamino)-2R-isobutylsuccinyl]amino-1-methoxy-3,4-dihydrocarbostyril;

3S-[4-(N-Hydroxyamino)-2R-isobutyl-3S-acetylthio-methylsuccinyl]amino-3,4dihydrocarbostyril;

3S-[s(N-Hydroxyamino)-2R-isobutyl-3S-methylsuccinyl]amino-1-methoxy-3,4dihydrocarbostyril;

3S-[4(-Hydroxyamino)2R-isobutylsuccinyl]amino-l-methoxymethyl-3,dihydrocarbostyril;

1-Carboxymethyl-3S-[4N-hydroxyamino)-2R-isobutyl-3S-methylsuccinyl]amino-3,4dihydrocarbostyril;

3S-[4-(N-Hydroxyamino)-2R-isobutylsuccinyl]amino-1-methoxyethoxymethyl-3,4-dihydrocarbostyril;

3S-[4(N-Hydroxyamino)-2R-heptylsuccinyl]amino-l-methoxy-3,4-dihydrocarbostyril;

7Chloro-3S-[(N-hydroxyamino)-2R-isobutylsuccinyl]amino1-methoxymethyl-3,4-dihydrocarbostyril;

3S-[4-(N-Hydroxyamino)-2R-isobutylsuccinyl]amino-l-methoxyethyl- 3,4-dihydrocarbostyril;

3S-[4(N-Hydroxyamino)-2R-isobutylsuccinyl]amino-1-methoxyethyl-6,7-methylenedioxy-3,4dihydrocarbostyril;

3R-[s(N-Hydroxyamino)2R-isobutylsuccinyl]amino-1-methoxyethyl-6,7-methylenedioxy-3,4-dihydrocarbostyril;

2-(R)-N-Hydroxy-2-[(4-methoxybenzenesulfonyl) (3-morpholin4-yl-3-oxopropyl)amino]-3-methyl-butyramide;

2-(R)-2-[(2-Benzylcarbamoylethyl)(4-methoxy-benzenesulfonyl)amino]-N-hydroxy-3-methylbutyramide;

2-(R)-N-Hydroxy-2-((4-methoxybenzenesulfonyl) (2-[(pyridin-3-ylmethyl)carbamoyl]ethyl)amino)-3-methylbutyramide;

2-(R)-N-Hydroxy-2-([4-methoxybenzenesulfonyl]-[2-(methylpyridin-3-ylmethylcarbamoyl)ethyl]amino)-3-methylbutyramide;

4-(3-[1-(R)-1-Hydroxycarbamoyl-2-methylpropyl)(4-methoxybenzenesulfonyl)amino]propionyl)piperazine-1carboxylic acid, tert-butyl ester;

2-(R)-N-Hydroxy-2-[(4-methoxybenzenesulfonyl)(3-oxo-3-piperazin-1-ylpropyl)amino)-3-methylbutyramide hydrochloride;

2-(R)-2-[(Benzylcarbamoylethyl)(4-methoxy-benzenesulfonyl)amino]-N-hydroxy-3-methylbutyramide;

2-(R)-N-Hydroxy-2-[(4-methoxybenzenesulfonyll[(2-morphoin-4-ylethylcarbamoyl)methyl]amino]-3-methylbutyramide;

2-(R)-N-Hydroxy-2-((4-methoxybenzenesulfonyl) ([(pyridin-3-ylmethyl)carbamoyl]methyl)amino)-3-methylbutyramide;

2-(R3,3,3,-Trifluoro-N-hydroxy-2-[(methoxy-benzenesulfonyl)(3- morpholinfyl-3-oxopropyl)amino]propionamide;

2-(RN-Hydroxy-2-((4-phenoxybenzenesulfonyl)[2-methylpyridin ylmethylcarbamoyl)ether]amino)-3-methylbutyramide;

4- [4Methoxybenzenesulfonyl)(3-morpholin4-yl-3-oxopropyl)amino]-1-methylpiperidene4carboxylic acid hydroxyamide;

2-(R)-N-Hydroxy-2-((4-methoxybenzenesulfonyl)-[3-(4-methylpiperazin- 1-yl)-3-oxopropyl]amino)-3-methylbutyramide;

2-(R)-2-[(2-Carboxyethyl)(4methoxybenzene-sulfonyl)amino]-N-hydroxy-3-methylbutyramide;

[(2-Carboxyethyl)(3,4-dimethoxybenzene-sulfonyl)amino]-N-hydroxy-

2-(R)-2-[(2-Carbamoylethyl)(4-methoxybenzene-sulfonyl)amino]-N-hydroxy-3-methylbutyramide;

2-(R), 3-(R)-3, N-Dihydroxy-2-[(4-methoxybenzenesulfonyl)(3-oxo-3-piperidin-1-ylpropyl)amino]-butyramide;

2-(R)-N-Hydroxy-2-((4-methoxybenzenesulfonyl)[3-(methylpyridin-3-ylmethylcarbamoyl)propyl]amino)-3-methylbutyramide;

2-(R)-N-Hydroxy-2-((4-methoxybenzenesulfonyl)[2-(methylcarboxymethylcarbamoylethyl]amino)-3-methyl-butyramide;

2-(R)-N-Hydroxy-2-((4-methoxybenzenesulfonyl)[(1-methylpiperidin4-ylcarbamoyl)methyl]amino)-3-methylbutyramide;

2-(R)-N-Cyclohexyl-N-hydroxy-2-((4methoxy-benzenesulfonyl)-[3-(4-methylpiperazin-1-yl)-3-oxopropyl]amino)-acetamide;

2-(R)-N-Hydroxy-2-[(methoxybenzenesulfonyl)(3-morpholin4yl-[3-oxopropyl)amino]4(morpholinyl)butyramide;

[4-N-Benzyloxyamino)-2(R)-isobutylsuccinyl]-L-leucyl-L-alanine ethyl ester;

[4N-Benzyloxyamino)-2(R)-isobutylsuccinyl]-3(RS)-aminolaurolactam;

Na-[4(N-Benzyloxyamino)-2(R)-isobutylsuccinyl]-Ne-N- benzyloxycarbonylglycyl)-L-lysyl-L-alanine ethyl ester;

[4(N-Hydroxyamino)-2(RS)-isobutylsuccinyl]-L-leucylglycine ethyl ester, [4-(N-Hydroxyawino)-2(RS)-isobutylsuccinyl]-L-leucylglycine isopentylamide;

[4-(N-Hydroxyamino)-2(RS)-isobutylsuccinyl]-L-valylglycine ethylamide;

[4-(N-Hydroxyamino)-2(RS)-isobutylsuccinyl]-L-leucylglycine ethylamide;

Na-[4-(N-Hydroxyamino)-2(RS)-isobutylsuccinyl]-Ne-tert butoxycarbonyl-L-lysylglycine ethylamide;

[4(N-Hydroxyamino)-2(RS)-isobutylsuccinyl]-O-methyl-L-tyrosinylglycine ethyl ester;

[(N-Hydroxyamino)-2(RS)isobutylsuccinyl]-O-methyl-L-tyrosinylglycine ethylamide;

[s(N-Hydroxyamino)-2(RS)-isobutylsuccinyl]-L-leucyl-L-alanine ethyl ester;

[4(N-Hydroxyamino)-2(RS)-isobutylsuccinyl]-L-leucylglycine isopentyl ester;

[4(N-Hydroxyamino)-2(R)-propylsuccinyl]-L-leucylglycine ethyl ester;

[s(N-Hydroxyamino)-2(RS)-sec.butylsuccinyl]-L-leucylglycine ethyl ester;

[4(N-Hydroxyamino)-2(R)-isobutylsuccinyl]-L-leucyl-L-alanine;

[4(N-Hydroxyamino)-2(RS)-isobutylsuccinyl]-L-leucylglycine methyl ester;

[4-(N-Hydroxyamino)2(RS)-isobutylsuccinyl]-L-leucylsarconsine ethyl ester;

[4-(N-Hydroxyamino)-2(RS)-isobutylsuccinyl]-L-leucyl-L-proline ethyl ester;

[4-(N-Hydroxyamino)-2(RS)-isobutylsuccinyl]-L-leucine-L-alanine isopropyl ester;

[4N-Hydroxyamino)-2(RS)-isobutylsuccinyl]-L-leucine-2-oxopropylamide;

[4-(N-Hydroxyamino)-2(RS)-isobutylsuccinyl]-L-leucine-2-methoxyethylamide;

[4-(N-Hydroxyamino)-2(RS)-isobutylsuccinyl]-Lleucine-2,2-dimethoxyethylamide;

Na-[4-(N-Hydroxyamino)2(R)isobutylsuccinyl]-Ne-glycyl-L-lysine methylamide;

Na-[4-(N-Hydroxyamino)-2(R)-isobutylsuccinyl]-Ne-(4-carboxybenzoyl)-L-lysl-L-alanine ethyl ester, Na-[4-(N-Hydroxyamino)-2(R)-isobutylsuccinyl]-Ne-(4carboxybenzoyl)-L-lysyl-L-aline;

[s(N-Hydroxyamino)-2(R)isobutylsuccinyl]-3(RS)-aminooctahydro-2H-azonin-2-one;

[4-(N-Hydroxyamino)-3(S)-methyl-2(R)-isobutyl-succinyl]-L-leucylglycine ethyl ester;

[(3-Aminophthalimido)methyl][(RS)4methyl-2-[[(S)3-methyl-1- (methylcarbamoyl)butyl]carbamoyl]pentyl]phosphinic acid;

[(RS)4-Methyl-2-[[(S)-3-methyl-l-(methyl-carbamoyl)butyl]carbamoyl]pentyl](1,8-naphthalenedi-carboximidomethyl)phosphinic acid;

[(R or S)4Methyl-2-[[(R or S)-2-oxo-3-azacyclotridecyljcarbamoyljpentyl)(1 .8-naphthalenedicarboximidomethyl)phosphinic acid;

N-[N-[(R or S)-2f[[[[N-[l-(Benzyloxy)carbonyl]-L-propyl]-L- leucyl]amino]methyl]hydroxyphosphinyl]-methyl]methylvaleryl]-L-leucyl]-L-alanine;

[[1,4-Dihydro-2,4-dioxo-3(2H)-quinazolinyl]-methyl][[(R or S)4 methyl-2-[[(R or S)-2-oxo-3-azacyclotridecyl]carbamoyl]pentyl]phosphinic acid;

N²-[(R)-Hydroxycarbamoylmethyl]4methylvaleryl]-N¹,3-dimethyl-L-valinamide;

N²-[2(R or S)-[[[(5-Bromo-2,3-dihydro-6hydroxy)-1,3-dioxo-1H-benz[dse]isoquinol-2-yl)methyl]-[(hydroxy)phosphinyl]methyl]4-methylvaleryl]-N1,3-dimethyl-L-valinamide;

N2-[(R or S)-[[(R)-(Amino)[(5-bromo-2,3-dihydro-6hydroxy-1,3-dioxo-1H-benz[d,e]isoquinol-2-yl)methyl](hydroxy)phosphinyl]methyl]) methylvaleryl]-N³,1-dimethyl-L-valinamide hydrobromide;

N²-[2(R or S)-[I(S)-(Hydroxycarbamoyl)ethyl-4-methylvaleryl]-N¹,3- dimethylvalinamide;

N²-[2(R)-(l(R or S)-(Hydroxycarbamoyl)-2-phthalimidoethyl]4methylvaleryl]-N¹,3-dimethyl-L-valinamide;

N²-[2(R)-[1(R or S)-(Hydroxycarbamoyl)4-(methoxyarbonyl)butyl]l methylvaleryl]-N¹,3-dimethyl-L-valinamide;

M²-[2(R)-[1(R or S(Hydroxycarbamoyl)-4-phenyl-butyl]l methylvaleryl]-N¹,3-dimethyl-L-valinamide;

N²-[2(R)-[1(R or S)-(Hydroxycarbamoyl)-2-succinimidoethyl]4-methylvaleryl]-N¹,3-dimethyl-L-valinamide;

4-[2(R)-[I(R or S)(Hydroxycarbamoyl)-2-phthalimidoethyl]4-methylvaleryl]morpholine;

4[2(R)-[1(R or S)-(Hydroxycarbamoyl)-2-phthalimidoethyl]4 methylvaleryl]tetrahydro-1,4thiazine;

1-[2(R)-[1(R or S)-(Hydroxycarbamoyl)-2-phthalimidoethyl]4 methylvaleryl]piperidinol;

1-[2(R)-[1(R or S)-(Hydroxycarbamoyl)-2-(1,2-dimethyl-3,5-dioxo-1,2,4 triazolidin4-yl)ethyl]A4-methylvaleryl]piperidine;

4-[2(R)-[1(R or S)-(Hydroxycarbamoyl)-2-(3-methyl-2,5-dioxo-1- imidazolidinyl)ethyl]methylvaleryl]tetrahydro-1,4-thiazine;

Hexahydro-2-[2(R)-[l(R or S)-(hydroxycarbamoyl)-2-phthalimidoethyl]-4-methylvaleryl]-N-methyl-3(S)-pyridazinecarboxamide;

1-[2(R)-(R or S)-(Hydroxycarbamoyl)-2-(3,4,4-trimethyl-2,5-dioxo-1- imidazolidinyl)ethyl]4-methylvaleryl]+piperidinol;

[4-(N-Hydroxyamino)-2(R or S)-heptylsuccinyl]-L-leucyl-L-leucine ethylamide;

[4-(N-Hydroxyamino)-2(R or S)-nonylsuccinyl]-L-leucyl-L-leucine ethylamide;

[4-(N-Hydroxyamino)-2(R or S)-heptyl-3(S)-methylsuccinyl]-L-leucyl-L-leucine ethylamide;

[4(N-Hydroxyamino)-2(R)-heptyl-3(R or S)-(phthalimidomethyl)succinyl]-L-leucyl-L-leucine ethylamide;

[4-(N-Hydroxyamino)-2(RS)-nonylsuccinyl]-L-tert.butylglycine methylamide;

[4(N-Hydroxyamino)-2(RS)-heptylsuccinyl]-L-phenylalanine methylamide;

[4(N-Hydroxyamino)-2(R)heptyl-3(R or S)-phthalimidomethyl)succinyl]-L-tert.butylglycine methylamide;

[4-(N-Hydroxyamino)-2(R)-heptyl-3(R or S)-(3-phenylpropyl)-succinyl]-L-leucyl-Lleucine ethylamide;

[4-(N-Hydroxyamino)-2(RS)-heptylsuccinyl]-L-leucine methylamide;

[4-(N-Hydroxyamino)-2(RS)-heptylsuccinyl]-L-leucine neopentylamide;

[4(N-Hydroxyamino)-2(RS)-heptylsuccinyl]-L-alanyl-L-leucine ethylamide;

[4(N-Hydroxyamino)-2(RS)-heptylsuccinyl-L-(Ne-phthaloyl)-lysyl-L leucine ethylamide;

[4(N-Hydroxyamino)-2(RS)-undecylsuccinyl]-L-leucyl-L-leucine ethylamide;

[s(N-Hydroxyamino)-2(RS)-heptylsuccinyl]-L-phenylatanyl-L-leucine ethylamide;

[4-(N-Hydroxyamino)-2(RS)-heptylsuccinyl]-L-nonalyl-L-leucine ethylamide;

[4-(N-Hydroxyamino)-2(RS)-heptylsuccinyl]-phenylalanine tert.butylamide;

[4(N-Hydroxyamino)-2(RS)-heptylsuccinyl]-L-tertbutylglycine methylamide;

[4-(N-Hydroxyamino)-2(RS)-heptylsuccinyl]-L-neopentylglycine methyl amide;

[4(N-Hydroxyamino)-2(RS)-heptylsuccinyl]-L-homophenylalanyl-L- leucine ethylamide;

[4(N-Hydroxyamino)-2(RS)-heptylsuccinyl]-L-cyclohexylalanine methylamide;

[4-(N-Hydroxyamino)-2(RS)-isooctylsuccinyl]-L-phenylalanine methylamide;

[4-(N-Hydroxyamino)-2(R)-heptylsuccinyl]-L-neonpentylglycine methylamide;

[4(N-Hydroxyamino)-2(R)-heptylsuccinyl]-(D or L)-P,>dimethylphenylalanine methylamide;

[4-(N-Hydroxyamino)-2(R)-heptylsuccinyl]-(D or L)-threo-p- methylphenylalanine methylamide;

[4-(N-Hydroxyamino)-2(R)-heptylsuccinyl]-DL-erthro->methylphenylalanine methylamide;

[4(N-Hydroxyamino)-2(R)-heptyl-3(R or S)-[(3-methyl-2,5-dioxo-1- imidazolidinyl)methyl]succinyl]-L-leucyl-L-leucine ethylamide;

N2-[3-Cyclobutyl-2(R or S)-[(hydroxycarbamoyl)-methyl]-propionyl]-N1,3-dimethyl-L-valinamide;

N2-[3-Cyclopropyl-2(R or S)-[(hydroxycarbamoyl)-methyl]-propionyl]-N1,3dimethyl-L-valinamide;

N2-[3-Cyclopentyl-2(R or S)-[(hydroxycarbamoyl)-methyl]-propionyl]-N1,3-dimethyl-L-valinamide;

N2-[3-Cyclopropyl-2(R)-[1(R or S)-[(hydroxy-carbamoyl)-2-(3,4,4-trimethyl-2,5-dioxo-1-imidazolidinyl)ethyl]propionyl1-N1,3-dimethyl-L-valinamide;

N2-[3-Cyclopropyl-2(R)-[l(R or S)-[(hydroxy-carbamoyl)4 phenylbutyl)]propionyl]-N1,3-dimethyl-L-valinamide;

N2-[3-Cyclobutyl-2(R)-[l(R or S)-(hydroxy-carbamoyl)4- phenylbutyl]propionyl]-N1,3-dimethyl-L-valinamide;

N2-[3-Cyclopentyl-2(R)-[l(R or S)-(hydroxycarbamoyl)-4- phenylbutyl]propionyl]-N1,3-dimethyl-L-valinamide;

1-[3-Cyclopropyl-2(R)-[I(R or S)-(hydroxy-carbamoyl)-2-(3,4,4-trimethyl-2,5-dioxo-1-imidazolidinyl)ethyl]propionyl]piperidine;

1-[3-Cyclopropyl-2(R)-[I(R or S)-(hydroxy-carbamoyl)2-(3,4,4 trimethyl-2,5-dioxo-1-imidazolidinyl1thyl]propionyl]4-piperidinol;

1-[3-Cyclobutyl-2(R)-[l(R or S(hydroxy-carbamoyl)-2-(3,4,4-trimethyl-2,5-dioxo-1-imidazolidinyl)ethyl]propionyl]piperidine;

1-[3-Cyclobutyl-2(R)-[1(R or S)-(hydroxy-carbamoyl)-2-(3,4,4trimethyl-2,5-dioxo-1-imidazolidinyl)ethyl]propionyl])piperidinol;

1-[3-Cyclopentyl-2(R)-[l(R or S)-(hydroxy-carbamoyl)-2-(3,4,4 trimethyl-2,5-dioxo-1-imidazolidinyl)ethyl)propionyl]4-piperidinol;

1-[3-Cyclopentyl-2(R)-[l(R or S)-(hydroxy-carbamoyl)-2-(3,4,4- trihethyl-2,5-dioxo-1-imidazolidinyl)ethyl]propionyl]piperidine;

3-[3-Cyclobutyl-2(R)-[l(R or S(hydroxycarbamoyl)-2-(3,4,4trimethyl- 2,5-dioxo-1-imidazolidinyl)ethyl]propionyl]-3-azabicyclo[3.2.2]nonane;

3-[3-Cyclopropyl-2(R)-[I(R or S)-(hydroxy-carbamoyl)-2-(3,4,4- trimethyl-2,5-dioxo-1-imidazolidinyl)ethyl]propionyl]-3-azabicyclo[3.2.2]nonane;

3-[3-Cyclopentyl-2(R)-[I(R or S)-(hydroxy-carbamoyl)-2-(3,4,4-trimethyl-2,5-dioxo-l-imidazolidinylethyl]propionyl]-3-azabicyclo[3.2.2]nonane;

1-[3-Cyclohexyl-2(R)-[l(R or S)-(hydroxycarbamoyl)-2-(3,4,4-trimethyl- 2,5-dioxo-1-imidazolidinyl)ethyl]propionyl]piperidine;

4-[3-Cyclopentyl-2(R)[1(R or S)-(hydroxycarbamoyl)2-(3,4,4-trimethyl-2,5-dioxo-1-imidazolidinyl)ethyl]propionyl]tetrahydro-1 ,4-thiazine;

4-[3-Cyclopentyl-2(R)[1(R or S)-(hydroxycarbamoyl)-2-(3,4,4-trimethyl- 2,5-dioxo-1-imidazolidinyl)ethyl]propionyl]tetrahydro-1,4thiazine S,S-dioxide;

4-[3-Cyclobutyl-2(R)-[I(R or S(hydroxycarbamoyl)-2-(3,4,4trimethyl- 2,5-dioxo-1-imidazolidinyl)ethyl]propionyl]tetrahydro-1,4-thiazine;

3-[3-Cyclopentyl-2(R)-[l(R or S)-(hydroxycarbamoyl)-2-(3,4,4-trimethyl-2,5-dioxo-1-imidazolidinyl)ethyl]propionyl]-5,5-dimethyl-N-propyl-[4(R)-thiazolidinecarboxamide;

4-[3-Cyclopentyl-2(R)-[I(R or S)-(hydroxycarbamoyl)-2-(3,4,4-trimethyl- 2,5-dioxo-1-imidazolidinyl)ethyl]propionyl]morpholine;

3-[3-Cyclopentyl-2(R)-[l(R or S)-(hydroxy-carbamoyl)-2-(3,4,4- trimethyl-2,5-dioxo-1-imidazolidinyl)ethyl]propionyl]-N,5,5-trimethyl-4(R)- thiazolidinecarboxamide;

4-[3-Cyclobutyl-2(R)-[l(R or S)-(hydroxy-carbamoyl)-2-(3,4,4-trimethyl-2,5-dioxo-1-imidazolidinyl)ethyl]propionyl]4phenylpiperazine;

4-[3-Cyclobutyl-2(R)-[1(R or S)-(hydroxyarbamoyl)-2-(3,4,4-trimethyl-2,5-dioxo-1-imidazolidinyl)ethyl]propionyl]morpholine;

1-[3-Cyclobutyl-2(R)-[I(R or S)-(hydroxy-carbamoyl)2-(3,4,4-trimethyl-2,5-dioxo-1-imidazolidinyl)ethyljpropionyl]pyrrolidine;

8-[3-Cyclobutyl-2(R)-[l(R or S)(hydroxycarbamoyl)-2-(3,4,4-trimethyl- 2,5-dioxo-1-imidazolidinyl)ethylpropionyl]-1,4dioxa-8-azaspiro[4,5]decane;

1-[3-Cyclobutyl-2(R)[1(R or S)-(hydroxycarbamoyl)-2-(3,4,4-trimethyl- 2,5-dioxo-1-imidazolidinyl)ethyl]propionyl]4methoxypiperidine;

1-[3-Cyclobutyl-2(R)-[I(R or S)-(hydroxycarbamoyl)-2-(3,4,4-trimethyl-2,5-dioxo-1-imidazolidinyl)ethyl]propionylloctahydroazocine;

1-[3-Cyclobutyl-2(R)-[l(R or S)-(hydroxycarbamoyl)-2-(5,5dimethyl-2,4- dioxo-3-oxazolidinyl)ethyl]propionyl]piperidine;

1-[3-Cyclobutyl-2(R)-[l(R or S)-(hydroxycarbamoyl)-2-(3,4,4-trimethyl-2,5-dioxo-1-imidazolidinyl)ethyl]propionyl]hexahydroazepine;

1-[3-Cyclobutyl-2(R)-[2-(hexahydro1,3-dioxopyrazolo[1,2-a][1,2,4]triazol-2-yl)-l(R or S)-(hydroxycarbamoyl)ethyl]propionyl]piperidine;

1-[3-Cyclobutyl-2(R)[l(R or S)-(hydroxycarbamoyl)-2-phthalimidoethyl]propionyl]piperidine;

2-[2(R)-[1(R or S)-(Hydroxycarbamoyl)4phenylbutyl]nonanoyl]-hexahydro-N-methyl-3(S)-pyridazinecarboxarnide;

N-Cyclohexyl-hexahydro-2-[2(R)1[(RS)-(hydroxycarbamoyl)X-phenylbutyl]nonanoyl]-3(S)-pyridazinecarboxamide;

Hexihydto-2- [2(R)- [l1(RS)(hydroxycarbamoyl)+phenylbutyl]nonanoyl]- N-(2,2,6,6tetramethyl4-piperidinyl)-3(S)-pyridazinecarboxamide;

1-[2(R)-[1(R or S)-Hydroxycarbamoyl)4-phenylbutyllnonanoyl]piperidine;

N2-[2(R)-[I(RS)-(Hydroxycarbwnoyl)2-(3,4,4-trimethyl-2,5-dioxo-l-imidazolidinyl)ethyl)nonanoyl]-N1-methyl-L-prolinamide;

1-[2(R)1I(R or S)(Hydroxycarbamoyl)-2-(3,4,4-trimethyl-²,5-dioxol-- imidazolidinyl)ethyl3nonanoyl]piperidine;

Hexahydro-2-[2(R)-I(R or S)-(hydroxycarbamoyl)-2-(3,4,4-trimethyl-²,5- dioxo1-imidazolidinyl)ethyl]nonanoyl]-N-methyl-3(S)-pyridazinecarboxide;

Hexahydro-2-[2(R or S)-[I(S)-(hydroxycarbamoyl)-3- phenylpropyl]undecanoyl3-N-methyl-³(S)-pyridazinecarboxamnide;

Hexahydro-2-[2(R or S)-[I(S)-(hydroxycarbamoyl)-3-phenylpropyllundecanoyll-N-methoxy-N-methyl -3(S)-pyridazinecarboxamide;

Hexahydro-2-[2(R or S)-[(I(S)-(hydroxycarbamoyl)-3-phenylpropyl]-undecanoyl]-N-(l,2,2,6,6pentamethyl4-piperidinyl)-3(S)-pyridazine-carboxamide;

Hexahydro-2-[2(R or S)-[I(S)-(hydroxycarbamoyl)ethyl]undecanoyl)-N-methyl-3(S)-pyridazinecarboxamide;

Hexahydro2-[2(R or S)-[I(S)(hydroxycarbamoyl)-3-phenylpropyljnonanoyl]-N-methyl-3(S)-pyridazinecarboxamide;

Hexahydro-2-[2(R or S)-[l(S)-(hydroxycarbamoyl)ethyl]nonanoyl]-N-methyl-3(S)-pyridazinecarboxamide;

1-[2(R or S)[I(S)-(Hydroxycarbamoylethyl]undecanoyl]piperidine;

1-[2-(R or S)-[1(S)-(hydroxycarbamoyl)3-phenylpropyl]undecanoylpiperidine;

Hexahydro-2-[2(R or S)-[I(S)-(hydroxycarbamoyl)-3-phenylpropyl]-undecanoyl]-N-(2,2,6,6tetramety14-piperidinyl)3(S)-pyrdazinecarboxamide;

Hexahydro-2-[2(R or S)-[I(S)-(hydroxycarbamoyl1ethyl]undecanoyl)-N-(₂,₂,6,6tetramethyl1piperidinyl)-3(S)-pyridazinecarboxamide;

1-[2(R or S)-t[(S)-(hydroxycarbamoyl)4-phenylbutyl]undecanoyl]-pipenidine;

4-[2(R or S)-[1(S)-(hydroxycarbamoyl)4phenylbutyl]undecanoyl]-morpholine;

1-(Benzyloxycarbonyl)-hexahydro-2-[2(R)-[(R or S)-(hydroxycarbamoyl)-4phenylbutyl]nonanoyl]-N-(a(S)-methylbenzyl)3(S)-pyridazinecarboxamide;

N-[(2R)-2-[2′-(Hydroxyamino)-2′-(oxoethyl]-5(carboxy)pentanoyl]-L-phenylalanine N-methylamide;

N-[(2R)-2-[2′-(Hydroxyamino)-2′-(oxoethyl]-6-(phenylmethoxy)hexanoyl]-Lphenylalanine N-methylamide;

N-[(2R)-2-[2′-(Hydroxyamino)-2′-(oxo)ethyl]-6(propylamino)-6-(oxo)hexanoyl]-L-phenylalanine N-methylamide;

N-[(2R)-2-[2′-(Hydroxyamino)-2″-(oxo)ethyl]-(6RS)-6-(hydroxy)heptanoyl]-L-phenylalanine N-methylamide;

(2S)-N-2-[(2′R)-2′-[2″-(Hydroxyamino)-2″-(oxo)ethyl]6′-(hydroxy)hexanoyl]amino-3,3-dimethylbutanoic acid N-methylamide;

(2S)-N-2-[(2′R)-2′-[2″-(Hydroxyamino)-2″-(oxo)ethyl]-6′-(phenylmethoxy)hexanoyl]amino-3,3-dimethylbutanoic acid N-methylamide;

N-[(2′R)-2-[2′-(Hydroxyamino)-2′-(oxo)ethyl]-6-(4′-oxobutylamino)hexanoyl]-L-phenylalanine N-methylamide;

2(S)-N-2-[(2′R)-2′-[2″-(Hydroxyamino)-2″-(oxo)ethyl]-6′-(oxo)-6′-(propylamino)hexanoyl]amino-3,3-dimethylbutanoic acid N-methylamide;

N-[(2R)-2-[(1 ′S)-1 ′-(Methyl)-2′-(hydroxyamino)-2′-(oxo)ethyl]-6-(phenylmethoxy)hexanoyl]-L-phenylalanine N-methylamide;

N-[(2R)-2-[(1′S)-1 ′-(Methyl)-2′-(hydroxyamino)2′-(oxo ethyl]-6(oxo)-6-(propylamino)hexanoyl]-L-phenylalanine N-methylamide;

(2S)-N-2[(2′R)-[(I″R)1″-(1,3-Dihydro-1,3-dioxo2H-isoindol-2-yl)methyl-2″-(hydroxyamino)-2″-(oxoethyl]-6′-(phenylmethoxy)hexanoyl]amino-3,3-dimethylbutanoic acid N-methylamide;

N-[(2R)-2-[2′-(Hydroxyamino)-2′-(oxo)ethyl]-6-(oxo)-6-(propylamino)hexanoyl]-L-phenylalanine N-2-phenylethylamide

(2S)-N-2-[(2′R)-2′-[(I″S)-l″-(Methyl)2″-(hydroxyamino)-2″-(oxoethyl]-6-(phenylmethoxy)hexanoyl]amino-3,3-dimethylbutanoic acid N-2-phenylethylamide;

(2S)-N-2-[(2′R)-2′-[(1″S)l″-(Methyl)2″- (hyroxyamino)-2″-(oxo)ethyl]-6′-(oxo)-6′-(propylamnino)hexanoyl]amino-3,3-dimethylbutanoic acid N-2-phenylethylamide;

(2S)-N-2-[(2′)-2′-[(I′S)l″-(Methyl)2Δ-(hydroxyamino)-2″-(oxo)ethyl]-6′-(oxo)-6′-(propylamino)hexanoyl]amino-3,3-dimethylbutanoic acid N-2-(4′-sulfamoyl)phenylethylamide;

(2S)-N-[(2′R)-2′-[2″-(Hydroxyamino)-2″-(oxo)ethyl]-6′-(phenylmethoxy)hexanoyl]amino-3-cyclohexylpropionic acid N-2-(4′-sulfamoyl)-phenylethylamide;

N-[2R)-2-[2′-(Hydroxyamino)-2′-(oxo)ethyl]-6′-(phenyl-methoxy)hexanoyl]-(3,5-dimethyl)phenylalanine N-2-(4′-sulfamoyl)phenylethylamide;

(2S)-N-2′-[(2′R)-2′-[2″-(Hydroxyamino)T2″-(oxo)ethyl]-6′[(4-methoxy)phenoxy]hexanoyl]amino-3,3-dimethylbutanoic acid N-2-(4′- sulfamoyl)phenylethylamide;

(2S)-N-2′[(2′R)-2′-[2′″-(Hydroxyamino)2″-(oxo)ethyl]-6′-[(4-methyl)phenoxy]hexanoyl]amino-3,3dimethylbutanoic acid N-2-(4′-sulfamoyl) phenylethylamide;

(2S)-N-2′-[(2′R)-2′-[2″-(Hydroxyamino)-2″-(oxo)ethyl]-6′-[(1-oxo)butylamino]hexanoyl]amino-3-cyclohexylpropionic acid N-2-(4′-sulfamoyl) phenylethylamide;

(2S)-N-2-[(2′R)-2′-[(1″S)-″-(Methyl)2″-(hydroxyamino2″-(oxoethyl]-6(phenylmethoxy)hexanoyl]amino-3,3-dimethylbutanoic acid N- methylamide;

(2S)-N-2-[(2′R)-2′-[(I″S)-1″-(2-Methylpropyl)-2″-(hydroxyamino)-2″-(oxoethyl]-6-(phenylmethoxy)hexanoyl1amino-3,3-dimethylbutanoic acid N-methylamide; N-[(2R)-2-[2′-(Hydroxyamino)-2′-(oxo)ethyl]-6-(phenoxy)-hexanoyl]-L-phenylalanine N-methylamide;

N-[(2R)-2-[2′-(Hydroxyamino)2′-(oxo)ethyl]-7-(phenoxy)heptanoyl] -L-phenylalanine N-methylamide;

(2S)-N-2′-[(2′R)-2′-[2″-(Hydroxyamino)-2″-(oxo)ethyl]-6′-(phenylmethoxy)hexanoyl]amino-3,3dimethylbutanoic acid N-2-phenylethylamide;

(2S)-N-2′-[(2′R)-2′-[2″-(Hydroxyamino)-2″-(oxo)ethyl]-6′-(phenylmethoxy)hexanoyl]amino-3,3-dimethylbutanoic acid N-2-(4′-sulfamoyl)-phenylethylamide;

N-[(2R)-2-[2′-(Hydroxyamino)-2′-(oxo)ethyl]-5-(phenylmethoxy)pentanoyl]-L-phenylalanine N-methylamide;

N-[(2R)-2-[2′-(Hydroxyamino)-2′-(oxoethyl]-7-(phenylmethoxy)heptanoyl]-L-phenylalanine N-methylamide;

N-[(2R)-2-[2′-(Hydroxyamino)-2′-(oxowethyl]-6-(phenyloxy)hexanoyl]-L-phenylalanine N-methylamide;

N-[(2R)-2-[2′-(Hydroxyamino)-2′-(oxo)ethyl]-7-[(phenyloxy)heptanoyl]-L-phenylalanine N-methylamide;

(2S)-N-2′-[(2′R)-2′-[2″-(Hydroxyamino)-2″-(oxoethyl]-6′-[(2-phenethylamino)-6′-(oxo)hexanoyl]amino-3,3-dimethylbutanoic acid N- methylamide;

(2SN-2′-[(2′R2′-[2″-(Hydroxyamino)-2″-(oxo)ethyl]-6′-[(4 methylphenoxy)hexanoyl]amino-3,3-dimethylbutanoic acid N-methylamide;

(2S)-N-2′-[(2′R)-2′-[2″-(Hydroxyamino)-2″-(oxo)ethyl]-6′-[(4chlorophenoxy)hexanoyl]amino-3,3 dimethylbutanoic acid N-methylamide;

(2S)-N-2′-[(2′R)-2′-[2″-(Hydroxyamino)-2″-(oxo)ethyl]-6′-[(3-methylphenoxy)hexanoyl]amino-3,3-dimethylbutanoic acid N-methylamide;

(2S)-N-2′-[(2′R2′-(carboxymethyl)-6′-(3-methylphenoxy)hexanoyl]amino-3,3dimethylbutanoic acid N-methylamide;

N-[(2R)-2-[2′-(Hydroxyamino)-2′7-(oxo)ethyl]-5-(carboxy)pentanoyl]-L phenylalanine N-methylamide;

N-[(2R2-[2′-(Hydroxyamino)-2′-(oxo)ethyl]-6-(phenylmethoxy)hexanoyl]-Lphenylalanine N-methylamide;

N-[(2R2-[2′-(Hydroxyamino)-2′-(oxo)ethyl]-6-(propylamino)-6-(oxo)hexanoyl]-L-phenylalanine N-methylamide;

N-[(2R)-2-[2′-(Hydroxyamino)-2′-(oxo)ethyl]-(6RS)-6-(hydroxy)heptanoyl]-L-phenylalanine N-methylamide;

(2S)-N-2-[(2′R)-2′-[2″-(Hydroxyamino)-2″-(oxo)ethyl]-6′-(hydroxy)hexanoyl]amino-3,3-dimethylbutanoic acid N-methylamide;

-64(2S)-N-2-[(2′R)-2′-[2″-(Hydroxyamino)-2″-(oxo)ethyl]-6′-(phenylmethoxy)hexanoyl]amino-3,3dimethylbutanoic acid N-methylamide;

N-[(2′R)-2-[2′-(Hydroxyamino)-2′-(oxo)ethyl]-6-(4′-oxobutylamino)hexanoyl]-L-phenylalanine N-methylamide;

2(S)-N-2-[(2′R)-2′-[2′-(Hydroxyamino)-2″-(oxo)ethyl]-6′-(oxo)-6′-(propylamino)hexanoyl]amino-3,3-dimethylbutanoic acid N-methylamide;

N-[(2R)-2-[(I′S)-1′-(Methyl)-2′-(hydroxyamino)-2′-(oxo)ethyl]-6-(phenylmethoxy)hexanoyl]-L-phenylalanine N-methylamide;

N-[(2R)-2-[(I′S)-1 ′-(Methyl)-2′-(hydroxyamino)-2′-(oxo)ethyl]-6-(oxo)-6-(propylamino)hexanoyl]-L-phenylalanine N-methylamide;

(2S)-N-2[(2′R)-[(1″R)-1″-(1,3-dihydro-1,3-dioxo-2H-isoindol-2-yl)methyl-2″-(hydroxyamino)-2″-(oxoethyl]-6′-(phenylmethoxy)hexanoyl]amino-3,3dimethylbutanoic acid N-methylamide;

N-[(2R)-2-[2′-(Hydroxyamino)-2′-(oxo)ethyl]-6-(oxo)-6-(propylamino)hexanoyl]-L-phenylalanine N-2-phenylethylamide;

(2S)-N-2-[(2′R)-2′-[(1″S)-l″-(Methyl)-2″-(hydroxyamino)-2″-(oxo)ethyl]-6-(phenylmethoxy)hexanoyl]amino-3,3-dimethylbutanoic acid N-2-phenylethylamide;

(2S)-N-2-[(2′R)-2′-[(l″S)-1″-(Methyl)-2″-(hydroxyamino)2″- (oxo)ethyl]-6′-(oxo)-6′-(propylamino)hexanoyl]amino-3,3-dimethylbutanoic acid N-2-phenylethylamide;

(2S)-N-2-[(2′R)-2′-[(l″S)-1″-(Methyl)-2′-(hydroxyamino)-2′- (oxo)ethyl]-6′-(oxo)-6′-(propylamino)hexanoyl]amino-3,3-dimethylbutanoic acid N-2-(4′-sulfamoyl)phenylethylamide;

(2S)-N-[(2′R)-2′-[2″-(Hydroxyamino)-2″-(oxo)ethyl]′-(phenylmethoxy)hexanoyl]amino-3-cyclohexylpropionic acid N-2-(4″-sulfamoyl)-phenylethylamide;

N-[(2R2-[2′-(1Hydroxyamino)-2′-(oxo)ethyl]-6′-(phenylmethoxy)hexanoyl]-L-(3,5dimethyl)phenylalanine N-2-(4′-sulfamoyl)phenylethylamide;

(2S)-N-2′-[(2′R)-2′-[2″-(ydroxyamino)-2″-(oxoethyI]-6′-[(4- methoxy)phenoxy]hexanoyl]amino-3,3-dimethylbutanoic acid N-2-(4′-sulfamoyl)phenylethylamide;

(2S)-N-2′-[(2′R)-2′-[2″-(Hydroxyamino)-2″-(oxo)ethyl]-6′-[(4-methyl)phenoxy]hexanoyl]amino-3,3methylbutanoic acid N-2-(4′-sulfamoyl)-phenylethylamide;

(2S)-N-2′-[(2′R)-2′-[2″-(Hydroxyamino)-2″-(oxo)ethyl]-6′-[(1-oxo)butylaminolhexanoyl]amino-3-cyclohexylpropionic acid N-2-(4′-sulfamoyl)-phenylethylamide;

(2S)-N-2-[(2′R)-2′-[(1″S)-1″-(Methyl)-2″-(hydroxyamino)2″-(oxoethyl]-6-(phenylmethoxy)hexanoyl]amino-3,3dimethylbutanoic acid N-methylamide;

(2S)-N-2-[(2′R)-2′-[(I″S)1″-(2-Methylpropyl)-2″-(hydroxyamino)-2″-(oxo)ethyl]-6-(phenylmethoxy)hexanoyl]amino-3,3-dimethylbutanoic acid N-methylamide;

N-[(2R2-[2′-(Hydroxyamino)-2′-(oxo)ethyi]-6-(phenoxy)hexanoyl]-L-phenylalanine N-methylamide;

N-[(2R)-2-[2′-(Hydroxyamino)-2′-(oxo)ethyl]-7-(phenoxy)heptanoyl]-L-phenylalanine N-methylamide;

(2S)-N-2′-((2′R)-2′-2″-((Hydroxyamino)-2″-(oxo)ethyl]-6′-(phenylmethoxy)hexanoyl]amino-3,3-dimethylbutanoic acid N-2-phenylethylamide;

(2S)-N-2′-[(2′R)-2′-[2″-(Hydroxyamino)-2″-(oxo)ethyl]-6′-(phenylmethoxy)hexanoylamino-3,3-dimethylbutanoic acid N-2-(4′-sulfamoyl) phenylethylamide;

N-C(2R)-2-[2′-(Hydroxyamino)-2′-(oxoethyl]-5-(phenylmethoxy)pentanoyl]-L-phenylalanine N-methylamide;

N-[(2R)-2-(2′-(Hydroxyamino)-2′-(oxo)ethyl]-7-(phenylmethoxy)heptanoyl]-L-phenylaIanine N-methylamide;

N-[(2R)-2-[2′-(Hydroxyamino)-2′-(oxoethyl1-6-(phenyloxy)hexanoyl]-phenylelanine N-methylamide;

N-[(2R)-2-12′-(Hydroxyamino)-2′-(oxo)ethyl]-7-[(phenyloxy)heptanoyl]-L-phenylalanine N-methylamide;

(2S)-N-2′-[(2′R)-2′-[2″-(Hydroxyamino)-2″-(oxo)ethyl]-6′-[(2-phenethylamino)-6′-(oxo)hexanoyl]amino-3,34dimethylbutanoic acid N-methylamide;

(2S)-N-2′-[(2′R)-2′-[2″-(Hydroxyamino)-2″-(oxoethyl]-6′-[(4-methylphenoxy)hexanoyl]amino-3,3-dimethylbutanoic acid N-methylamide;

(2S)-N-2′-[(2′R)-2′-[2″-(Hydroxyamino)-2″-(oxo)ethyl]-6′-[(4 chlorophenoxy)hexanoyl]amino-3,3-dimethylbutanoic acid N-methylamide;

(2S)-N-2′-[(2′R)-2′-[2″-(Hydroxyamino)2″-(oxo)ethyl]-6′-[(3-methylphenoxy)hexanoyl]amino-3,3-dimethylbutanoic acid N-methylamide;

(2S)-N-2′-[(2′R)-2′-(Carboxymethyl)-6′-(3-methylphenoxy)hexanoyl]amino-3,3-dimethylbutanoic acid N-methylamide;

(3R,10S)-5-Methyl-3-(9-oxo-1,8-diazatricyclo[106.1.0)nonadeca- 12(19),13(18),14,16-tetraen-10-ylcarbamoyl)hexanoic acid;

(3R,10S)-N-Hydroxy-5-methyl-3-(9-oxo-1,8- diazatricyclo[10.6.1.0]nonadeca-12(19),13(18),14,16tetraen-10- ylcarbamoyl)hexanamide;

(3R,11S)N-Hydroxy-5-methyl-3-(I-oxo-1,9iazatricyclo[I 1.6.1.0]eicosa- 13(20),14(19),15,17-tetraen-1 1-ylcarbamoyl)hexanamide;

(3R,9S)-5-Methyl-3-(8-oxo-1,7-diazatricyclo[9.6.1 .0]octadeca- 11(18),12(17),13,15-tetraen-9-ylcarbamoyl)hexanoic acid;

(3R,9S)-N-Hydroxy-5-methyl-3-(8-oxo-1,7-diazatricyclo[9.6.1.0]octadeca- 11(18),12(17),13,15-tetraen-9-ylcarbamoyl)hexanamide;

(10S)-[4-Methyl-2-(9-oxo-1,84iazatricyclo[10.6.1.0]nonadeca- 12(19),13(18),14,16-tetraen-10-ylcarbamoyl)pentyl]-(quinolin-2- ylthiomethyl)phosphinic acid;

(3R,10S)-N-Hydroxy-5-methyl-2-methoxycarbonyl-3- (9-oxo1,8- diazatricyclo[lO.6. 1.0)nonadeca-12(19),13(18),14,16-tetraen-10- ylcarbamoyl)hexanamide;

N-(4Methyl-2-carboxymethylpentanoyl)-L-leucine-N′-(4 methoxycarbonylphenyl)carboxamide;

N-(4-Methyl-2-(N″′-hydroxycarbamoyl)methylpentanoyl)-L-leucine-N′-(4- methoxycarbonylphenyl)carboxamide;

N-(4Methyl-2-(N″-hydroxycarbamoyl)methylpentanoyl)-L-leucine-N′-(4- carboxyphenyl)carboxamide;, N-(4Methyl-2-(N″-hydroxycarbamoyl)methylpentanoyl)-L-tryptophan- N′-(carboxyphenyl)carboxamide;

N-(4-Methyl-2-(N″-hydroxycarbamoyl)methylpentanoyl)-L cyclohexylglycine-N′-(4-methoxycarbonylphenyl)carboxamide;

N-(4Methyl-2-(N″-hydroxycarbamoyl)methylpentanoyl)-L--leucine-N.′- (4methoxycarbonylphenyl)carboxamide;

(3R,10S)-6-Biphenyl4-yl)-3-(9-oxo-1,8diazatricyclo[10.6.1.0]onadeca- 12(19),13(18),14,16-tetraen-10-ylcarbamoyl)hexanoic acid;

(3R,10S)-3-(9-Oxo-1,8-diazatricyclo[10.6.1.0]nonadeca- 12(19),13(18),14,16-tetraen-10-ylcarbamoyl)-5-(thiophen-2-yl)pentanoic acid;

(3R,10S)-3-Cyclopentyl-3-(9-oxo-1,8-diazatricyclo[10.6.1.0]nonadeca- 12(19),13(18),14,16-tetraen-10-ylcarbamoyl)propionic acid;

(3R,10S)4Cyclopentyl-3-(9-oxo-1,8-diazatricyclo[10.6. 1.0]nonadeca- 12(19),13(18),14,16-tetraen-10-ylcarbamoyl)butanoic acid;

(3R,10S)-4-Cyclopropyl-3-(9-oxo-1,8-diazatricyclo[10.6.1.0]nonadeca- 12(19),13(18),14,16-tetraen-10-ylcarbamoyl)butanoic acid;

(3R,10S)-5-Methyl-3-(9-oxo-1,8-diazatricyclo[10.6.1.0]nonadeca- 12(19),13(18),14,16-tetraen-10ylcarbamoyl)hexanoic acid;

(3R,10S)-N-Hydroxy-5-methyl-3-(9-oxo1,8- diazatricyclo[10.6.1.0]nonadeca-12(19),13(18),14,16-tetraen-10- ylcarbamoyl)hexanamide;

(3R, I1S)-N-Hydroxy-5-methyl-3-(l 0oxo-1,9- diazatricyclo[1 1 .6.1.0]eicosa-13(20),14(19),15,17-tetraen-1 1- ylcarbamoyl)hexanamide;

(3R,9S)-N-5-Methyl-3-(8-oxo1,7-diazatricyclo[9.6.1.0]octadeca- 11(18),12(17),13,15-tetraen-9-ylcarbamoyl)hexanoic acid;

(3R,9S)-N-Hydroxy-5-methyl-3-(8-oxo-1,7diazatricyclo[9.6.1.0]octadeca- 11(18),12(17),13,15-tetraen-9-ylcarbamoyl)hexanamide;

(10S)-2-Mercaptomethyl-1methyl-N-(9-oxo-1,8- diazatricyclo[10.6.1.0]nonadeca-12(19),13(18),14,16-tetraen-10- ylcarbamoyl)pentanamide;

(10S)-2-Acetylthiomethylmethyl-N-(9-oxo-1,8- diazatricyclo[10.6.1.0]nonadeca-12(19),13(18),14,16-tetraen-10- ylcarbamoyl)pentanamide;

(3R,10S)-2-(Methanesulfonamidomethyl)5-methyl-3-(9-oxo-1,8- diazatricyclo[10.6. 1.0lnonadeca-12(19),13(18),14,16-tetraen-10- ylcarbamoyl)hexanoic acid;

(3R,10S)-2-(3-Ethylureidomethyl)-5-methyl-3-(9-oxo-1,8- diazatricyclo[l0.6.1.O]nonadeca-12(19),13(18),14,16-tetraen-10- ylcarbamoyl)hexanoic acid;

(3R,9S)-N-Hydroxy-2-hydroxy-5-methyl-3-(8-oxo-1,7- diazatricyclo[9.6.1.0]octadeca-1 1(18),12(17),14,16-tetraen-9- ylcarbamoyl)hexanamide or its (2S,3R,9S) stereoisomer;

(3R,10S)-N-Hydroxy-5-methyl-2-methoxycarbonyl-3-(9-oxo-1,8- diazatricyclo[I0.6.1.0]nonadeca-12(19),13(18),14,16-tetraen-10-ylcarbamoyl)- hexanamide;

(3R,9S)-5-Methyl-3-(8-oxo4-oxa- 1,7-diazatricyclo[9.6. 1.0]octadeca- 11(18),12,14,16-tetraen-9-ylcarbamoyl)hexanoic acid;

(3R,9S)-3-Cyclobutylmethyl-N-(8-oxoAoxa-1,7- diazatricyclo[9.6.1.0]octadeca-1 1(18),12,14,16-tetraen-9-ylcarbamoyl)succinamic acid;

(3R,9S)-3-(8-Oxo oxa-1,7-diazatricyclo[9.6.1.0]octadeca- 11(18),12,14,16-tetraen-9-ylcarbamoyl)-5-phenoxy-pentanoic acid;

(3R,9S)-5-(4-Chlorophenoxy)-3-(8-oxo4oxa-1,7- diazatricyclo[9.6. 1 .0]octadeca-1 1(18),12,14,16-tetraen-9-ylcarbamoyl)pentanoic acid;

(3R,9S)-5-(4Chlorophenoxy)-3-(8-oxo4oxa-1,7- diazatricyclo[9.6.1.0]octadeca-I 1(18),12,14,16-tetraen-9-ylcarbamoyl)pentanoic acid ethyl ester;

(3R,9S)-3-(8-Oxo-1,7-diazatricyclo[9.6.1.O]octadeca-1 1(18),12,14,16- tetraen-9-ylcarbamoyl)pentanoic acid ethyl ester;

(3R,9S)-6-(4-Hydroxy-phenyl)-3-(8-oxo4oxa-1,7- diazatricyclo[9.6.1 .0]octadeca-1 1(18),12,14,16-tetraen-9-ylcarbamoyl)hexanoic acid;

(3R,9S)-3-(8-OxoA4oxa-1,7 diazatricyclo[9.6. 1.0]octadeca- 11(18),12,14,16-tetraen-9-y1carbamoyl)-6-pyridin y1-hexanoic acid;

(3R,9S)-6-[4-(3-Hydroxy-propoxy)-phenyl]-3-(8-oxo4oxa-1,7- diazatricyclo[9.6.1.0]octadeca-1 1(18),12,14,16-tetraen-9-ylcarbamoyl)hexanoic acid;

(3R,9S)-3-(8-Oxo4foxa-1,7-diazatricyclo[9.6.1.0]octadeca- 11(18),12,14,16-tetraen-9-ylcarbamoyl)-5-(4phenoxy-phenyl)pentanoic acid;

(3R,9S)-6-[4(2-Hydroxy-ethoxy)-phenyl]-3-(8-oxo4oxa-1,7- diazatricyclo[9.6.1.0]octadeca-11(18),12,14,16-tetraen-9-ylcarbamoyl)hexanoic acid;

(3R,9S)-3-(8-Oxofoxa-1,7-diazatricyclo[9.6.1.0]octadeca- 11(18),12,14,16-tetraen-9-ylcarbamoyl)-6-[4-(2-pyrrolidin-1-yl- ethoxyphenyl]hexanoic acid;

(3R,9S)-6-(4Methoxy-phenyl)-3-(8-oxo4oxa-1,7-diazatricyclo[9.6.1.0]- octadeca-1 1(18),12,14,1 &tetraen-9-ylcarbamoyl)hexanoic acid;

(3R,9S)4-[4-(2-Methoxy-ethoxy)phenyl]-3-(8-oxo4-oxa-1,7- diazatricyclo[9.6.1.0]octadeca-1 1(18),12,14,16-tetraen-9-ylcarbamoyl)hexanoic acid;

(3R,9S)-3-(8-Oxo4oxa-1,7-diazatricyclo[9.6.1.0]octadeca- 11(18),12,14,16-tetraen-9-ylcarbamoyl)-5-phenyl-pentanoic acid;

(3R,9S)3-(8-Oxo4oxa- 1,7-diazatricyclo[9.6. 1.0]octadeca- 11(18),12,14,16-tetraen-9-ylcarbamoyl)-6-phenyl-hexanoic acid;

(3R,9S)-6-(3-Hydroxy-pheny1l)3-(8-ox o4oxa-1,7-diazatricyclo[9.6.1.01- octadeca-1 1(18),12,14,16-tetraen-9-ylcarbamoyl)hexanoic acid;

(3R,9S)-3-(8-Oxooxa-1,7-diazatricyclo[9.6.1.0]octadeca- 11(18),12,14,16-tetraen-9-ylcarbamoyl)-6-[4-(3-piperidin-1-yl- propoxy)phenyl]hexanoic acid;

(3R,9S)-6-[4(3-Dimethylamino-propoxy)-phenyl]-3-(8exoA4oxa-1,7- diazatricyclo[9.6.1.0]octadeca-1 1(18),12,14,16-tetraen-9-ylcarbamoyl)hexanoic acid;

(3R,9S)-6-[4-(2-Dimethylamino-ethoxy)-phenyl]-3-(8oxo4oxa-1,7- diazatricyclo[9.6.1.0]octadeca-1 1(18),12,14,16-tetraen-9-ylcarbamoyl)hexanoic acid;

(3R,9S)-6-(4-Cyano-phenyl)-3-(8-oxo4-oxa-1,7-diazatricyclo[9.6.1.0]- octadeca-1 1(18),12,14,16-tetraen-9-ylcarbamoyl)hexanoic acid;

(3R,9S6-Naphthalen-2-yl-3-(8-oxo4xa-1,7-diazatricyclo[9.6.1.0]- octadeca-1 1(18),12,14,16-tetraen-9-ylcarbamoyl)hexanoic acid;

(3R,9S)-3-(8-Oxo4-oxa-1,7-diazatricyclo[9.6.1.0]octadeca- 11(18),12,14,16-tetraen-9-ylcarbamoyl)-6-(4-pyrolo-1-y1)hexanoic acid;

(3R,9S)-6-(4-Hydroxy-3-methyl-phenyl)-3-(8-oxo4-oxa-1,7- diazatricyclo[9.6.1.0]octadeca-1 1(18),12,14,16-tetraen-9-ylcarbamoyl)hexanoic acid;

(3R,9S6-(4Benzyloxy-phenyl)-3-(8-oxooxa-1,7- diazatricyclo[9.6.1.0]octadeca-1 1(18),12,14,16-tetraen-9-ylcarbamoyl)hexanoic acid;

(3R,9S)-6-[4(4Aminobutoxy-pheny1)]-3-(8-ox0o4-oxa-1,7- diazatricyclo[9.6.1.0]octadeca-1 1(18),12,14,16-tetraen-9-ylcarbamoyl)hexanoic acid;

(3R,9S)-5-(4Methoxy-phenyl)-3-(8-oxo-4-oxa-1,7-diazatricyclo[⁹.⁶.1.0]- octadeca-1 1(18),12,14,16-tetraen-9-ylcarbamoyl)pentanoic acid;

(3R,9S)-6-(4Amino-phenyl)-3-(8-oxo4oxa-1,7-diazatricyclo[⁹.⁶.1.0]- octadeca-1 1(18),12,14,16-tetraen-9-ylcarbamoyl)hexanoic acid;

(3R,9S)-3-(8-Oxofoxa-1,7-diazatricyclo[9.6.1.0]octadeca- 1 1(18),12,14,16-tetraen-9-ylcarbamoyl)-6-[4yridin4- ylmethoxy)phenyl]hexanoic acid;

(3R,9S)(4-Acetylaminophenyl)-3-(8-oxo4-oxa-1,7- diazatricyclo[9.6.1.0]octadeca- 11(18),12,14,16tetraen-9-ylcarbamoyl)hexanoic acid;

NcL[[3-(N-Hydroxycarbamoyl)-4-methylthio-2-propoxymethyl]butylyi]- N,O-dimethyltyrosine amide;

Na[[3-(N-Hydroxycarbamoyl)-4-isopropylthio2- propoxymethyl]butylyl]-N,O-dimethyltyrosine amide;

NQ-[[3-(N-Hydroxycarbamoyl)-2-propylthio]butylyl]-N,O- dimethyltyrosine amide;

N-[N-(l-Phosphono-3-phenylpropyl)-(S)-leucyl]-(S)-phenylalanine-N- methylamide;

N-[N-(1-Phosphono-3-(4-bromo-1,8-naphthalene-dicarboximido)propyl)- (S)-leucyl]-(S)phenylalanine methylamide;

N-[N-(1-Phosphono-3-(benzyloxycarbonylamino)propyl)-(S)-leucyl]-(S)- phenylalanine methylamide;

N-N-(l-Phosphono-3-(2-hydroxyphenyl)propyl)-(S)-leucyl]-(S)- phenylalanine methylamide;

N-[N-(1-Phosphono-3-(methylmercapto)propyl)-(S)-leucyl]-(S)- phenylalanine-N-methylamide;

N-[N-(1-Phosphono-3-(methylsulphinyl)propyl)-(S)-leucyl]-(S)- phenylalanine-N-methylamide;

N-[N-(1-Phosphono-3-(methylsulphonyl)propyl)-(S)-leucyl]-(S)- phenylalanine-N-methylamide;

N-[N-(l-Phosphono-3-(1,8-naphthalenedicarboximido)propyl)-(S)-leucyl]- (S)-tryptophan-N-methylamide;

N-[N-(I-Phosphono-3-(1,8-naphthalenedicarboximido)propyl)-(S)leucyl]- (S)-lysine-N-methylamide;

N-[N-(1-Phosphono-3-(1,8-naphthalenedicarboximido)propyl)-(S)leucyl]- (-)-aminoazacyclotridecan-2-one;

N-[N-(1-Phosphono-3-(1,8-naphthalenedicarboximido)propyl)-(S)leucyl]- (S)-lysine-N-(aminoethyl)amide;

N-[N-(1-Phosphono-3-(1,8-naphthalenedicarboximido)propyl)-(S)-leucyl]- (S)-lysine-N-(ethylpyrrolidine)amide;

N-(N-(1-Phosphono-3-(1,8-naphthalenedicarboximido)propyl)-(S)-leucyl]- (S)-lysine-N-(ethyl-N-methylpiperazine)amide;

N-[N-(1-Phosphono-3-[8-(7,9-dioxo-8-azaspiro[4,5]decyl)]propyl)-(S)- leucyl]-(S)-phenylalanine-N-methylamide; and

N-[N-(I-Phosphono-3-[8-(7,9-dioxo-8-azaspiro[4,5]decyl)]propyl)(S) leucyl]-(S)-lysine-N-methylamide.

As noted above, numerous inhibitors of matrix metalloproteinases are known. A large number of inhibitors are characterized as hydroxamic acid-based and/or carboxylic acid-based compounds. Typical of such compounds are those described in the following references, all of which are incorporated herein by reference, since all of the disclosed compounds can be used in the method of this invention.

| | |
|---|---|
| U.S. Pat. No. 4,599,361 | (Searle) |
| EP-A-2321081 | (ICI) |
| EP-A-0236872 | (Roche) |
| EP-A-0274453 | (Bellon) |
| WO 90/05716 | (British Biotechnology) |
| WO 90/05719 | (British Biotechnology) |
| WO 91/02716 | (British Biotechnology) |
| WO 92/09563 | (Glycomed) |
| U.S. Pat. No. 5,183,900 | (Glycomed) |
| U.S. Pat. No. 5,270,326 | (Glycomed) |
| WO 92/17460 | (Smith-Kline Beecham) |
| EP-A-0489577 | (Celltech) |
| EP-A-0489579 | (Celltech) |
| EP-A-0497192 | (Roche) |
| U.S. Pat. No. 5,256,657 | (Sterling Winthrop) |
| WO 92/13831 | (British Biotechnology) |
| WO 92/22523 | (Research Corporation Technologies) |
| WO 93/09090 | (Yamanouchi) |
| WO 93/09097 | (Sankyo) |
| WO 93/20047 | (British Biotechnology) |
| WO 93/24449 | (Celltech) |
| WO 93/244 | (Celltech) |
| EP-A-0574758 | (Roche) |
| WO 94/02447 | (British Biotechnology) |
| WO 94/02446 | (British Biotechnology) |
| WO 97/27174 | (Shionogi) |

An especially preferred group of compounds to be employed in the present method are those described in WO 95/35275 and WO 95/35276, both of which are incorporated herein by reference. Typical compounds from within these groups to be employed include:

N-Hydroxy-2-[[(2-(4methoxy-phenoxy)ethyl-(toluenewsulfonyl)-amino]-acetamide;

N-Hydroxy-2-[(4phenoxy-ethyl)-toluenesulfonyl) amino]-acetamide;

N-Hydroxy-2-[(4methoxy-benzenesulfonyl)-nonyl-amino]-acetamide;

2-[-Decyl-(toluenesulfonyl)-amino]-N-hydroxy-acetamide;

2-Benzyl-(octane-l-sulfonyl)-amino]-N-hydroxy-acetamide;

N-Hydroxy-2-[(2-methoxy-benzyl)-(octane- l-sulfonyl)-amino]-acetamide;

2-[(2-Ethoxy-benzyl)-(octane-1-sulfonyl)-amino]-N-hydroxy-acetamide;

N-Hydroxy-2-[(naphthalen-2-yl-methyl)-(octane-1-sulfonyl)-amino]- acetamide;

2-[(4-Chloro-benzyl)-(octane-1-sulfonyl)-amino]-N-hydroxy-acetamide, and salts, solvates, or hydrates thereof.

Another class of matrix metalloproteinase inhibitors are aryl sulfonamides of the formula

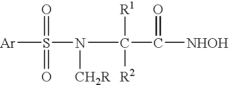

where Ar is carbocyclic or heterocyclic aryl, and R, R¹, and R² include hydrogen, alkyl, aryl, heteroaryl, amino, substituted and disubstituted amino. These compounds are disclosed in European Patent Number 0606046, incorporated herein by reference. Specific compounds to be employed in the present method include:

N-Hydroxy-2-[[4-methoxybenzenesulfonyl](isobutyl) amino]acetamide;

N-Hydroxy-2-[[4methoxybenzenesulfonyl](cyclo-hexylmethyl)amino]- acetamide;

N-Hydroxy-2-[[4methoxybenzenesulfonyll(cyclo hexyl)amino]acetamide;

N-Hydroxy-2-[[4methoxybenzenesulfonyl](phenethyl) amino]acetamide;

N-Hydroxy-2-[[4-methoxybenzenesulfonyl](3-methylbutyl)amino]acetamide;

N-Hydroxy-2-[[4methoxybenzenesulfonyl](sec-butyl)amino]acetamide;

N-Hydroxy-2-[[4methoxybenzenesulfonyl](tert-butyl)amino]acetamide;

N-Hydroxy-2-[[4-methoxybenzenesulfonyl)(4-fluorobenzyl)amino]acetamide;

N-Hydroxy-2-[[4-methoxybenzenesulfonyl](4 chlorobenzyl)amino]acetarnide;

N-Hydroxy-2-[[4-methoxybenzenesulfonyll (isopropyl)-amino]acetamide;

N-Hydroxy-2-[[4-methoxybenzenesulfonyl](4-methylbenzyl)amino]acetamide;

4-N-Hydroxy-carbamoyl]-4-[[4-methoxybenzene-sulfonyl(benzyl)-amino]-l-[dimethylaminoacetyl]-piperidine hydrochloride;

4-N-Hydroxy-carbamoyl]4-[[methoxybenzene-sulfonyl(benzyl)- amino]-1-[3-picolyl]-piperidine dihydrochloride;

4-N-Hydroxy-carbamoyl]l[[4-methoxybenzene-sulfonyl(benzyl)-amino]-1-[carbomethoxymethyl]-piperidine hydrochloride;

4N-Hydroxy-carbamoyl][[4-methoxybenzene-sulfonyl(benzyl)-amino]-l-piperidine trifluoroacetate;

4N-Hydroxy-carbamoyl]-4-[[4-methoxybenzene-sulfonyl(benzyl)-amino]-1-[t-butoxycarbonyl]-piperidine;

4-N-Hydroxycarbamoyl]+[[4-methoxybenzene-sulfonyl(benzyl)-amino]-1-[methylsulfonyl]-piperidine;

N-Hydroxycarbamoyl]A[[4methoxybenzene-sulfonyl(benzyl)-amino]-l-[4-picoly]-piperidine hydrochloride;

N-Hydroxycarbamoyl]4[[4methoxybenzene-sulfonyl(benzyl)amino]-1-[morpholinocarbonyl]-piperidine hydrochloride;

N-(t-Butyloxy)-2-[[4-methoxybenzenesulfonyl (benzyl)amino]-2-[2-(4-morpholino)ethyl]acetamide;

N-Hydroxy-2-[[4-methoxybenzenesulfonyl](isobutyl) amino-2-(2-(4-morpholinoethyl]acetamide;

N-Hydroxy-2-[[4-methoxybenzenesulfonyl](2-picoly)- amino-2-(2-(4-morpholinoethyl]acetamide dihydro-chloride;

N-Hydroxy-2-[[4-methoxybenzenesulfonyl) (3-picolyl)amino]-2-[2-(4-morpholino)ethyl]acetamide dihydrochloride;

N-Hydroxy-2-[[4-methoxybenzenesulfonyl](2-methyl-thiazol-4-ylmethyl)amino]-2-[2-(4-morpholino) ethyl]acetamide dihydrochloride;

N-Hydroxy-2-[[4-methoxybenzenesulfonyl] benzyl)amino]-2-[2-(4 thiomorpholino]ethyl]acetamide;

N-Hydroxy-2-[[4methoxybenzenesulfonyl] (benzyl)aminoJ-2-[2-(4-methylthiazol4iylmethyl] acetamide;

N-Hydroxy-2-[[4methoxybenzenesulfonyl (benzyl)amino]-2-[(6-chloropiperonyl]acetamide;

N-Hydroxy-2-[[4methoxybenzenesulfonyl (benzyl)amino]-2-((1-pyrazolyl)methyl]acetamide;

N-Hydroxy-2-[[4methoxybenzenesulfonyl (3-picolyl)amino]-2-[3-picolyl]acetamide;

N-Hydroxy-2-[[4methoxybenzenesulfonyl(benzyl)-amino]-2-[(1-methyl-4-imidazolyl)methyl]acetamide hydrochloride;

N-Hydroxy-2-[[4methoxybenzenesulfonyl(isobutyl) amino]-2-[(1-methyl-4-imidazolyl)methyl]acetamide hydrochloride;

N-Hydroxy-2-[[4methoxybenzenesulfonyl](3-picolyl) aminoi-2-[(1-methylimidazolyl) methyl]acetamide hydrochloride;

N-Hydroxy-2-[[4-methoxybenzenesulfonyl(2-picolyl) amino]-2-[(1-methylimidazolyl)methyl]-acetamide hydrochloride; and

N-Hydroxy-2-([4-methoxybenzenesulfonyl] (2-methylthiazol4-ylmethyl)amino-2-[(1-methyl4imidazolyl)methyl]acetamide hydrochloride.

Another group of small peptide matrix metalloproteinase inhibitors are described in U.S. Pat. Nos. 5,270,326, 5,530,161, 5,525,629, and 5,304,604 (incorporated herein by reference). The compounds are hydroxamic acids, for example compounds of the formula

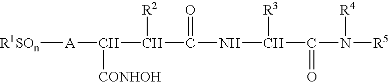

where R¹, R², R³, R⁴, and R⁵ can be alkyl, A includes a hydrocarbon chain, and n is 0 to 2. Typical compounds to be employed in the instant method include the following:

N-[2-Isobutyl-3-(N′-hydroxycarbonylamido)-propanoyl]-D-tryptophan methylamide;

N-[2-Isobutyl-3-(N′-hydroxycarbonylamido)-propanoyl]-N-methyl-L-tryptophan methylamide;

5 N-[2-Isobutyl-3-(N-hydroxycarbonylamido)-propanoyl]-L3-(2-naphthyl)-alanine methylamide;

N-[2-Isobutyl-3-(N′-hydroxycarbonylamido)-propanoyl]-L-tryptophan 2-hydroxyethylamide;

N-[2-Isobutyl-3-(N′-hydroxycarbonylamido)-propanoyl]-L-tryptophan amylamide;

N-[2-Isobutyl-3-(N′-hydroxycarbonylamido)-propanoyl]-L-tryptophan piperidinamide;

N-[2-Isobutyl-3-(N′-hydroxycarbonylamido)-propanoyl-L-tryptophan dodecylamide;

N-[2-Isobutyl-3-(N′-hydroxycarbonylamido)-propanoyl]-Ltryptophan(S)-methylbenzylamide;

N-[L2-Isobutyl-3-(N′-hydroxycarbonylamido)-propanoyl]-L-tryptophan(6-phenylmethoxycarbonyl-amino-hexyl-1)amide;

2S-Hydroxy-3R-[1S-(3-methoxy-2,2-dimethyl-propylcarbamoyl)-2,2-dimethyl-propylcarbamoyl]-5-methyl-hexanohydroxamic acid;

2S-Hydroxy-3R-[1S-(methylcarbamoyl)-2,2-dimethyl-propylcarbamoyl]-6-(4chloro)phenyl-hexanohydroxamic acid;

2S-Hydroxy-3R-[1S-(methylcarbamoyl)-2,2-dimethyl-propyl-carbamoyl]octanohydroxamic acid;

2S-Hydroxy-3R-[IS-(pyridin-2-ylmethylcarbamoyl)-2,2-dimethyl-propyl-carbamoyl]-5-methyl-hexanohydroxamic acid;

2S-Hydroxy-3R-[1S-(pyridin-3-ylmethylcarbamoyl)2,2-dimethyl-propyl-carbamoyl]-5-methyl-hexanohydroxamic acid;

2S-Hydroxy-3R-[1S-(pyridinAylmethylcarbamoyl)-2,2-dimethyl-propyl-carbamoyl]-5-methyl-hexanohydroxamic acid;

2S-Hydroxy-3R-[1S-(methylcarbamoyl)-2,2-dimethyl-propylcarbamoyl]-4-methoxy-butanohydroxamic acid;

2S-Hydroxy-3R-[1S-(methylcarbamoyl)-2,2-dimethyl-propylcarbamoyl]-4-benzyloxy-butanohydroxamic acid;

2S-Hydroxy-3R-[1S-(methylcarbamoyl)-2,2-dimethyl-propylcarbamoyl]-4-benzylthio-butanohydroxamic acid;

2S-Hydroxy-3R-[IS-(methylcarbamoyl)2,2-dimethyl-buten-3-ylcarbamoyl]-5-methyl-hexanohydroxamic acid;

2S-Hydroxy-3R-[1S-(tert-butylcarbamoyl)-2,2dimethyl-propyl-carbamoyl]-5-methyl-hexanohydroxamic acid;

2S-Hydroxy-3R-[1S-(N,Ndimethylarbamoyl)-2,2-dimethyl-propyl-carbamoyl]-5-methyl-hexanohydroxamic acid;

2S-Hydroxy-3R-[1 S-(3-hydroxy-2,2-dimethyl-propylcarbamoyl)-2,2-dimethyl-propyIcarbanoyl]-5-methyl-hexanohydroxamic acid;

2S-Hydroxy-3R-[IS-(methylcarbamoyl)-2,2-dimethyl-propylcarbamoyl]-6-phenyl-hexanohydroxamic acid;

2S-Hydroxy-3R-[IS-(methylcarbamoyl)-2,2-dimethyl-butylcarbamoyl]-5-methyl-hexanohydroxamic acid;

[″(-Hydroxyamino)2R-isobutylsuccinyl]-L-phenylalanine-N-(2-hydroxyethyl)-amide;

[″(-Hydroxyarnino)2R-isobutylsuccinyl]-L-phenylalaninyl-proline;

[4-(N-Hydroxyamino)2R-isobutylsuccinyl]-Lphenylalanine-N-(2-hydroxyethyl)-N-methylamide;

[4-(N-Hydroxyarnino)-2R-isobutylsuccinyl]-L-phenylalaninyl-D-prolinol;

[4(N-Hydroxyamino)2R-isobutylsuccinyl] -L-phenylalaninyl-L-prolinol;

[4-(N-Hydroxyamino)-2R-isobutylsuccinyl]-L-phenylalanine-N-(5-N-methyl-pentylcarboxamide)amide;

[″(-Hydroxyarnino)-2R-isobutylsuccinyl]-L-phenyIalanine-N-(2-ethylthioethyl)amide;

[4-(N-Hydroxyamino)-2R-isobutylsuccinyl]-L-phenylalanine-N-(2-methoxyethyl)amide;

[4-(N-Hydroxyarnino)-2R-isobutylsuccinyl]-L-phenylalanine-N-(2-N-acetylethyl)amide;

[4-(N-Hydroxyamino)-2R-isobutylsuccinyl]-L-phenylalanine-N-(3-(2-pyrrolidone)propyl)amide;

[4-(N-Hydroxyamino)-2R-isobutylsuccinyl]-L-phenylalanine-N-(3-(2-pyrrolidone)propyl)amide sodium salt;

[4-(N-Hydroxyamino)-2R-isobutylsuccinyl]-L-phenylalanine-N-(2-acetoxyethyl)amide;

[4(-Hydroxyamino)-2R-isobutyl-3S-methylsuccinyl]-L-phenylalanine- N-(3-(2-pyrrolidone)propyl)amide;

[4-(N-Hydroxyamino)-2R-isobutyl-3S-methylsuccinyl]-L-phenylalanine-N-methyl-N-(2-hydroxyethyl)amide;

[4-(N-Hydroxyamino)-2R-isobutyl-3S-methylsuccinyl]-I-phenylalanine-N-(2-hydroxyethyl)amide;

[4-(N-Hydroxyamino)-2R-isobutyl-3S-methylsuccinyl] -L-phenylalaninyl-D-prolinol;

[4(N-Hydroxyamino)-2R-isobutyl-3S-methylsuccinyl]-L-phenylalanine-N-(3-(2-pyrrolidone)propyl)amide sodium salt;

[4-(N-Hydroxyamino)-2R-isobutyl-3S-methylsuccinyl]-L-phenylalanine-N-(3-(2-pyrrolidone)propyl)amide;

[4(N-Hydroxyamino)-2R-isobutyl-3S-methylsuccinyl]-L-phenylalanine- N-(3-(2-pyrrolidone)propyl)amide or a salt thereof;

N²[4-(N-Hydroxyamino)-3S-(4-hydroxyphenylthiomethyl)-2R- isobutylsuccinyll-N⁶tert-butyloxycarbonyl-L-lysine-NImethylamide;

N²-[4(N-Hydroxyamino)-3S-(4-hydroxyphenylthiomethyl)-2R- isobutylsuccinyl]-N6tert-butyloxycarbonyl-N⁶-(4-hydroxyphenylthiotnethyl)-L-lysine-N¹-methylamide;

N²-[4-(N-Hydroxyamino)-3S-(2-thienylthiomethyl)2R-isobutylsuccinyl]-N6tert-butyloxycarbonyl-L-lysine-Nlmethylamide;

N²-[4-(N-Hydroxyamino)-3S-(4-hydroxyphenylthiomethyl)-2R- isobutylsuccinyll-O-tert-butyl-L-threonine-N¹-methylamide;

N²-[4-(N-Hydroxyamino)-3S-(4-hydroxyphenylthiomethyl)-2R-isobutylsuccinyl]-L-glutamine-NI,N⁵-dimethylamide;

N²-[4(N-Hydroxyamino)-3S-(4hydroxyphenylsulphonylmethyl)-2R-isobutylsuccinyl]-N⁶-acetyl-L-lysine-N¹-methylamide;

3R-(3-Methoxycarbonyl-1S-methylcarbamoyl-propylcarbamoyl}5-methyl-2S-2-propenyl-hexanohydroxamic acid;

3R-(1S-Methylcarbamoyl-2-thien-2-yl-ethylcarbamoyl)-5-methyl-2S-2-propenyl-hexanohydroxamic acid;

3R-(3-Methyl-IS-methylcarbamoyl-butylcarbamoyl)-5-methyl-2S-2- propenyl-hexanohydroxamic acid;

2S-[1S-Methylcarbamoyl-2-oxadiazol-5-yl-ethylcarbamoyl)-5-methyl-2S- 2-propenyl-hexanohydroxamic acid;

[6(N-Hydroxyamino)-2R-isobutylsuccinyl]-L-(4-oxymethylcarboxylic acid)phenylalanine-N-methylamide;

[4(N-Hydroxyamino)-2R-isobutylsuccinyl]-L-(4-oxymethylcarboxy-N- methylamide)phenylalanine-N-methylamide;

[4(N-Hydroxyamino)-2R-isobutylsuccinyl]-L-(4-oxymethylcarboxy-beta-alanine)phenylalanine-N-methylamide;

[((N-Hydroxyamino)-2R-isobutylsuccinyl]-L-(4-oxymethylcarboxyglycine)phenylalanine-N-methylamide;

[4(N-Hydroxyamino)-2R-isobutylsuccinyl]-L-(4-oxymethylcarboxy-N-benzylamide)phenylalanine-N-methylamide;

[4(N-Hydroxyamino)-2R-isobutylsuccinyl]-L(4-cyano)phenylaanine-N-methyl amide;

f[(-Hydroxyarnino)-2R-isobutylsuccinyl]-L-(4-acetamido)phenylalanine-N-methylamide;

[4-(N-Hydroxyamino)-2R-isobutylsuccinyl]-L-(4-oxymethylcarboxamide)-henylalanine-N-methylamide;

[4-(N-Hydroxyamnino)-2R-isobutyl-3S-(2-thienylthiomethylsuccinyl]-L-(4-N-acetylamino)-henylalanine-N-methylamide;

[4-(N-Hydroxyamino)2R-isobutyl-3S-(2-thienylthiomethylsuccinyl]-L(4 N-methylsuccinylamide)phenylalanine-N-methylamide;

[4(N-Hydroxyamino)-2R-isobutyl-3S-(4-aminophenylthiomethyl) succinyt]-L-(4N-(methylsuccinylamide)phenylalanine-N-methylamide;

[4-(N-Hydroxyamino)-2R-isobutyl-3S-(4 aminophenylthiomethylsuccinyl]-L-(4N-(4(4oxobutanoic acid)aminophenylalanine-N-methylamide;

[4(N-Hydroxyamino)-2R-isobutyl-3S-(4-hydroxyphenylthiomethyl)-succinyl]-L-(4-N-methylsuccinylamido)phenylalanine-N-methylamide;

[4-(N-Hydroxyamino)-2R-isobutyl-3S-(4hydroxyphenylthiomethyl) succinyl]-L,(4-N-(4(4-oxobutanoic acid)aminophenylalanine-N-methylamide;

[4(N-Hydroxyamino)-2R-isobutyl-3S-(2-thienylthiomethyl)-succinyl]-L (4-oxymethylcarboxymethyl)phenylalanine-N-methylamide;

(4-(N-Hydroxyamino)-2R-isobutyl-3S -(2-thienylthiomethyl)-succinyl]-L (4-N-(oxymethylcarboxylic acid)phenylalanine-N-methylamide;

[4(N-Hydroxyamino)-2R-isobutyl-3S-(2-thienylthiomethyl)-succinyl]-L 4-oxymethylcarboxyglycyl methyl ester)-phenylalanine-N-methylamide;

[4(N-Hydroxyamino)-2R-isobutyl-3S-(2-thienylthiomethyl)-succinyl]-L 4-oxymethylcarboxyglycine)phenylalanine-N-methylamide;

[4-(N-Hydroxyamino)-2R-isobutyl-3S-methyl-succinyl]-IA (oxymethylcarboxyglycyl methyl ester)-phenylalanine-N-methylamide;

[s(-Hydroxyamino)-2R-isobutyl-3S-methyl)-succinyl]-L4- (oxymethylcarboxyglycine)-phenylalanine-N-methylamide;

[4(-Hydroxyamino)-2R-isobutylsuccinyl]I-LA-oxymethylnitrile) phenylalanine-N-methylamide;

[s(N-Hydroxyamino)2R-isobutylsuccinyl]-L-3-(1-(2- methyloxycarbonyl)ethyl)4methoxyphenylalanine-N-methylamide;

[4-(N-Hydroxyamino)-2R-isobutylsuccinyl]-L3-(hydroxymethyl)4 methoxyphenylalanine-N-methylamide;

[s(N-Hydroxyamino)-2R-isobutylsuccinyl]-L-3-methyl4 methoxyphenylalanine-N-methylamide;

2-[Benzyl-(octane-l-sulfonyl)-amino]-N-hydroxy-acetamide;

N-Hydroxy-2-[(2-methoxy-benzyl)(octane-1-sulfonyl)amino]-acetamide;

2-[(2-Ethoxy-benzyl)-(octane-1-sulfonyl)-amino]-N-hydroxy-acetamide;

N-Hydroxy-2-[(naphtialen-2-yl-methyl)-(octane-1-sulfonyl)-amino]-acetamide;

2-[(4Chloro-benzyl)-(octane-1-sulfonyl)-amino]-N-hydroxy-acetamide;

N²[3S-Hydroxy4-(Nhydroxyamino)-2R-isobutylsuccinyl]-L-leucine-NI-methylamide;

N²-[3S-Hydroxy4(N-hydroxyamino)-2R-isobutylsuccinyl)]-5-methyl-L-glutamnic acid-N¹-methylamide;

N²-[3S-Hydroxy4-(N-hydroxyamino)-2R-isobutylsuccinyl)]-L-phenylalanine-N¹-methylamide;

[4-(N-Hydroxyamino)-2R-isobutyl-3S-(thienylthiomethyl)succinyl]-L-phenylalanine-N-methylamide;

[4-(N-Hydroxyamnino)-2R-isobutyl-3S-phenylthiomethyl)succinyl]-L phenylalanine-N-methylamide;

2S-(4Methoxyphenylsulfanylmethyl)-3R-(2-phenyl-lS-methylcarbamoyl-ethylcarbamoyl)-5-methyl-hexanohydroxamic acid;

2S-(3-Chlorophenylsulfanylmethyl)-3R-(2-phenyl-1S-methylcarbamoyl-ethylcarbamoyl)-5-methyl-hexanohydroxamic acid;

2S-(Phenylsulfanylmethyl)-3R-(2-phenyl-1S-(pyrid-3- ylmethylcarbamoyl)-ethylcarbamoyl)-5-methyl-hexanohydroxamic acid;

2S-(3-Methylphenylsulfanylmethyl)3R-(2-phenyl-lS-methylcarbamoyl-ethylcarbamoyI)5-methyl-hexanohydroxamic acid;

2S-(Ibien-2-ylsulfanylmethyl)-3R-(2-(4-carboxymethoxyphenyl)-1S-methylcarbamoyl-ediylcarbamoyl)-5-methyl-hexanohydroxamic acid;

2S-(Tien-2-ylsulfanylmethyl)-3R-(2-phenyl-1S-(pyrid-3-yhmethylcarbamoyl)-ethylcarbamoyl)5-methyl-hexanohydroxamic acid;

2S-(4-Hydroxyphenylsulfanylmethyl)-3R-(2-phenyl-1S-(pyrid-3-ylmethylcarbamoyl)-ethylcarbamoyl)-5-methyl-hexanohydroxamic acid;

2S-(Thien-2-ylsulfanylmethyl)-3R-(2-naph-2-yl-1S-methylcarbamoyl-ethylcarbamoyl)-5-methyl-hexanohydroxamic acid;

2S-(4Hydroxyphenylsulfanylmethyl)-3R-(2R-hydroxy-IS- methylcarbamoyl-propylcarbamoyl)-5-methyl-hexanohydroxamic acid;

2S-(4-Hydroxyphenylsulfanylmethyl)-3R-(5-acetamido1S-methylcarbamoyl-pentylcarbamoyl)-5-methyl-hexanohydroxamic acid;

2S-(4-Hydroxyphenylsulfanylmethyl)-3R-(3-[1,1-dimethylethoxycarbonyl]-lS-methylcarbamoyl-propylcarbamoyl)-5-methyl-hexanohydroxamic acid;

2S-(Thien-2-ylsulfonylmethyl)-3R-(2-phenyl-1S-methylcarbamoyl-ethylcarbamoyl)-5-methyl-hexanohydroxamic acid;

3S-(2-[4-Acetamido-phenyl]-1S-methylcarbamoyl-ethylcarbamoyl)-5-methyl-hexanohydroxamic acid;

2S-(4Phthalimido-butyl)-3R-(3-methyl-1 S-ethoxycarbonylmethylcarbamnoyl-butylcarbamoyl)-5-methyl-hexanohydroxainic acid;

3R-(2-[4-Methoxy-phenyl]-1S-methylcarbamoyl-ethylcarbamoyl)-2S,5-dimethyl-hexanohydroxamic acid;

3R-(2-Phenyl-lS-[2-oxo-pyrolid-1-yl]-propylcarbamoyl-ethylcarbamoyl)-5-methyl-hexanohydroxamic acid;

3R-(2-[4-Methoxy-phenyl]-lS-methylcarbamoyl-ethylcarbamoyl)-5-methyl-hexanohydroxamic acid;

3R-(2-Phenyl-1S-[pyrid-3-ylmethylcarbamoyl]-ethylcarbamoyl)-5-methyl-hexanohydroxamic acid;

3R-(2,2-Dimethyl-IS-methylcarbamoyl-propylcarbamoyl)-5-methyl- hexanohydroxamic acid;

Isobutylmalonoyl-L-alanine-furfurylamide hydroxamate;

2-Isobutyl-3-carbonyl-3′-(4acetylaniline)propionic acid;

N-Benzyloxycarbonyl-a-phosphonoglycyl-L-alanine furfurylamide;

[4(N-Hydroxyamino)-2R-isobutyl-3S-(phenylthiomethyl)succinyl]-L phenylalanine-N-methylamide;

[4(N-Hydroxyamino)-2R-isobutyl-3S-(4-methoxyphenylthiomethyl)-succinyl]-L-phenylalanine-N-methylamide;

[4(N-Hydroxyamino)-2R-isobutyl-3S-(4hydroxyphenylthiomethyl)- succinyl]-L-phenylalanine-N-methylamide;

[4-(N-Hydroxyamino)-2R-isobutyl-3S-(2,Sdimethylphenylthiomethyl)-succinyl]-L-phenylalanine-N-methylamide;

[4(N-Hydroxyamino)-2R-isobutyl-3S-(3-bromophenylthiomethyl)-succinyl]-L-phenylalanine-N-methylamide;

[s(N-Hydroxyamino)2R-isobutyl-3S-(3chlorophenylthiomethyl)-succinyl]-L-phenylalanine-N-methylamide;

[s(-Hydroxyamino)-2R-isobutyl-3S-(3-methylphenylthiomethyl)-succinyl]-L-phenylalanine-N-methylamide;

[s(N-Hydroxyamino)-2R-isobutyl-3S-(4-(N-acetyl)-amino-phenylthiomethyl)succinyl]-L-phenylalanine-N-methylamide;

[4-(N-Hydroxyamino)-2R-isobutyl-3S-phenyisulphinylmethylsuccinyl]-LD phenylalanine-N-methylamide;

3R-(3-Methoxycarbonyl-1S-methylcarbamoyl-propylcarbamoyl)-5-methyl-2S-phenylsulfanylmethyl- hexanohydroxamic acid;

3R-(3-Methoxycarbonyl-IS-methylcarbamoyl-propylcarbamoyl)-5-methyl-2S-(thien-2-ylsulfanylmethyl)-hexanohydroxamic acid;

2S-(4-Methoxy-phenylsulfanylmethyl)-3R-(3-methoxycarbonyl-1S-methylcarbamoyl-propylcarbamoyl)-5-methyl-hexanohydroxamic acid;

2S-(4-Amino-phenylsulfanylmethyl)-3R-(3-methoxycarbonyl-1S-methylcarbamoyl-propylcarbamoyl)-5-methyl-hexanohydroxamic acid;

2S-(Ethylsulfanylmethyl)-3R-(3-methoxycarbonyl-IS-methylcarbamoyl-propylcarbamoyl)-5-methyl-hexanohydroxamic acid;

2S-(Acetylsulfanylmethyl)-3R-(3-methoxycarbonyl-1S-methylcarbamoyl-propylcarbamoyl)-5-methyl-hexanohydroxamic acid;

2S-(Benzylsulfanylmethyl)-3R-(3-methoxycarbony-1IS-methylcarbamoyl-propylcarbamoyl)5-methyl-hexanohydroxamic acid;

2S-(tert-Butylsulfanylmethyl)-,3R-(3-methoxycarbonyl-1S- methylcarbamoyl-propylcarbamoyl)-5-methyl-hexanohydroxamic acid;

2S-Thiomethyl-3R-(3-methoxycarbonyl-1S-methylcarbamoyl-propylcarbamoyl)-5-methyl-hexanohydroxamic acid;

2S-(4-Hydroxy-phenylsulfanylmethyl)-3R-(2-tert-butoxycarbonyl-IS-methylcarbamoyl-ethylcarbamoyl)-5-methyl-hexanohydroxamic acid;

2S-(4-Hydroxy-phenylsulphinylmethyl)-3R-(3-methoxycarbonyl-1S-methylcarbamoyl-propylcarbamoyl)-5-methyl-hexanohydroxamic acid;

2S-(4-Hydroxy-phenylsulphonylmethyl)-3R-(3-methoxycarbonyl-1S-methylcarbamoyl-propylcarbamoyl)-5-methyl-hexanohydroxamic acid;

[s(N-Hydroxyamino)-2R-isobutylsuccinyl]-L-phenylalanine-N-[1-(2-amninoethyl)-pylfolidine]amide;

[4(N-Hydroxyamino)-2R-isobutylsuccinyl)-L-phenylalanine-N-[1-(3-aminopropyl)-2(RS)-methylpiperidine]amide;

[4-(N-Hydroxyamino)-2R-isobutylsuccinyl]-Iphenylalanine-N-[2-(2-amninoethyl)-1-methylpyrrole]amide;

[s(N-Hydroxyamino)-2R-isobutylsuccinyl]-L-phenylalanine-N-(3-aminomethylpyridine)amide;

[4(N-Hydroxyamino)-2R-isobutylsuccinyl]-L-phenylalanine-N-(2-aminomethylpyridine)amide;

[s(N-Hydroxyamino)-2(RS)-isobutylsuccinyl]-Lphenylalanine-N-(4 aminomethylpyridine)amide;

[s(N-Hydroxyamino)2(RS)-isobutylsuccinyl]-Lphenylalanine-N-(1-(3-aminopropyl)-imidazole)amide;

[4(N-Hydroxyamino)2(RS)-isobutylsuccinyl]-L-phenylalanine-N-(2-aminomethylbenzimdazole)amide;

[4(N-Hydroxyamino)-2R-isobutyl-3S-methylsuccinyl]-L-phenylalanine-N-[4(2-aminoethyl)-morpholino]amide;

[l(N-Hydroxyamino)-2R-isobutylsuccinyl]-L-phenylalanine-N-[4(2-aminoethyl)-morpholine]amide;

[s(-Hydroxyamino)-2(R,S)-isobutylsuccinyl]-L-phenylalanine-N-[2-(2-aminoethyl)-pyridine]amide;

[4(N-Hydroxyamino)-2(R ,S)isobutylsuccinyl]-L-phenylalanine-N-[4(2-aminopropyl)-morpholine]amide;

[4(N-Hydroxyamino)-2R-isobutylsuccinyl]-L-phenylalanine-N-(3-aminomethylpyridine)amide hydrochloride; and

[4(N-Hydroxyamino)-2R-isobutylsuccinyl]-L-phenylalanine-N-[4(2-aminoethyl)-morpholine]amide hydrochloride.

In a preferred embodiment, tricyclic butyric acid derivatives which are inhibitors of matrix metalloprotienases are employed to treat or prevent heart failure and ventricular dilatation according to this invention. A preferred group of tricyclic butyric acid derivatives are defined by the formula:

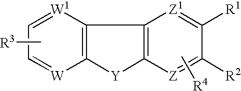

wherein one of R¹ or R² is

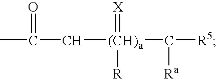

wherein X is O,

N-OR⁶ wherein R⁶ is hydrogen,

—(CH₂)ₙ-aryl wherein n is zero or an integer of 1 to 5, alkyl, or

—(CH₂)ₙ-cycloalkyl wherein n is as defined above, or

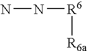

wherein R⁶ and R^{6a} are each the same or different and each is as defined above for R⁶;

R and R^{a} are each the same or different and each is hydrogen,

—(CH₂)ₙ-aryl wherein n is as defined above,

—(CH₂)ₙ-heteroaryl wherein n is as defined above,

—(CH₂)ₚ-R⁷—(CH₂)_{q}-aryl wherein R⁷ is 0 or S and p or q is each zero or an integer of 1 to 5 and the sum of p+q equals an integer of 5,

—(CH₂)ₚ-R⁷—(CH₂)_{q}-heteroaryl

wherein p, q, and R⁷ are as defined above,

alkyl,

—(CH₂)ₙ-cycloalkyl wherein n is as defined above, or

—(CH₂)—NH₂ wherein r is an integer of 1 to 9;

a is zero or an integer of 1 to 3;

R⁵ is OH,

OR⁶ wherein R⁶ is as defined above,

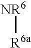

wherein R⁶ and R^{6a} are each the same or different and are as defined above for R⁶, or

NH-OR⁶ wherein R⁶ is as defined above;

R³ and R⁴ are each the same or different and each is hydrogen,

alkyl,

NO₂,

halogen,

OR⁶ wherein R⁶ is as defined above,

CN,

CO₂R⁶ wherein R⁶ is as defined above,

SO₃R⁶ wherein R⁶ is as defined above,

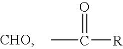

wherein R is as defined above,

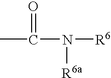

wherein R⁶ and R^{6a} are each the same or different and are as defined above for R⁶, or

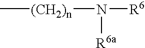

wherein R⁶ and R^{6a} are each the same or different and are as defined above for R⁶;

W, W¹, Z, and Z¹ are each the same or different and each is CR³ wherein R³ is as defined above, or

N providing only one of W or W¹ is

N and/or only one of Z or Z¹ is N; and Y is

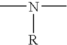

wherein R is as defined above,

—S—(O)ₘ- wherein m is zero or an integer of 1 or 2,

—CH₂,

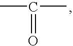

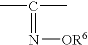

wherein R⁶ is as defined above,

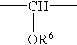

wherein R⁶ is as defined above,

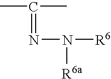

wherein R⁶ and R^{6a} are the same or different and are as defined above for R⁶,

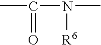

wherein R⁶ is as defined above,

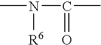

wherein R⁶ is as defined above,

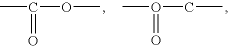

—CH₂—O—,

—O—CH₂—,

—CH2—S(O)ₘ— wherein m is as defined above,

—S(O)ₘ—CH₂— wherein m is as defined above,

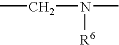

wherein R⁶ is as defined above,

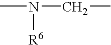

wherein R⁶ is as defined above,

—CH═N—, or

—N═CH—;

with the proviso that when X is O, and R⁵ is not NH—OR⁶, at least one of R or R^{a} is not hydrogen; and corresponding isomers thereof; or a pharmaceutically acceptable salt thereof. Typical compounds from this class include:

4-Dibenzofuran-2-yl-4-hydroxyimino-butyric acid; 2-(2-Dibenzofuran-2-yl-2-hydroxyimino-ethyl)- 4-methyl-pentanoic acid;

2-(2-Dibenzofuran-2-yl-2-hydroxyimino-ethyl)-5-phenyl-pentanoic acid;

4-Dibenzofuran-2-yl-4-hydroxyimino-2-phenethyl-butyric acid;

5-(4-Chloro-phenyl)-2-(2-dibenzofuran-2-yl-2-hydroxyimino-ethyl)-pentanoic acid;

2-(2-Dibenzofuran-2-yl-2-hydroxyimino-ethyl)-5-(4-fluoro-phenyl)-pentanoic acid;

2-(2-Dibenzofuran-2-yl-2-hydroxyimino-ethyl)-5-(4-methoxy-phenyl)-pentanoic acid;

2-(2-Dibenzofuran-2-yl-2-hydroxyimino-ethyl)-5-p-tolyl-pentanoic acid;

3-(Dibenzofuran-2-yl-hydroxyimino-methyl)-5-methyl-hexanoic acid;

3-(Dibenzofuran-2-yl-hydroxyimino-methyl)-6-phenyl-hexanoic acid;

3-(Dibenzofuran-2-yl-hydroxyimino-methyl)-5-phenyl-pentanoic acid;

6-(4-Chloro-phenyl)-3-(dibenzofuran-2-yl-hydroxyimino-methyl)-hexanoic acid;

3-(Dibenzofuran-2-yl-hydroxyimino-methyl)-6-(4-fluoro-phenyl)-hexanoic acid;

3-(Dibenzofuran-2-yl-hydroxyimino-methyl)-6-(4-methoxyphenyl)-hexanoic acid; and

3-(Dibenzofuran-2-yl-hydroxyimino-methyl)-6-p-tolyl-hexanoic acid; and corresponding isomers thereof; or a pharmaceutically acceptable salt thereof.

Tricyclic butyric acids having an a-amino substituent are defined by the formula:

wherein X is O, NOR₉, S, OH, SH, or;

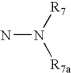

R₇ and R₇ₐ independently are

hydrogen,

C₁-C₂₀ alkyl or substituted C₁-C₂₀ alkyl,

(CH₂)₀₋₆₋aryl,

(CH₂)₀₋₆₋heteroaryl, or

(CH₂)₀₋₆₋cycloalkyl;

R₁ and R₂ independently are

hydrogen,

C₁-C₂₀ alkyl or substituted C₁-C₂₀ alkyl,

halo,

NO₂,

CN,

CHO,

COR⁶,

COOR₆,

SO₃R₆,

OR₆,

CONR4R₅,

(CH₂)₀₋₆-aryl,

(CH₂)₀₋₆-heteroaryl, or

(CH₂)₀₋₆-cycloalkyl;

R₆ is hydrogen,

C₁-C₂₀ alkyl or substituted C₁-C₂₀ alkyl;

aryl is phenyl or substituted phenyl;

R₃ is hydroxy,

O—C₁-C₂₀ alkyl or substituted O—C₁-C₂₀ alkyl,

O—(CH₂)₁₋₃ aryl, or

NHOR6;

R₄ and R₅ independently are hydrogen,

C₁-C₂₀ alkyl or substituted C₁-C₂₀ alkyl,

(CH₂)₀₋₆-aryl,

(CH₂)₀₋₆-heteroaryl; or one of R₄and R₅ is hydrogen and the other is:

COR₈,

CSR₈,

CONR₈R₉,

CSNR₈R₉,

COOR₈,

COSR₈,

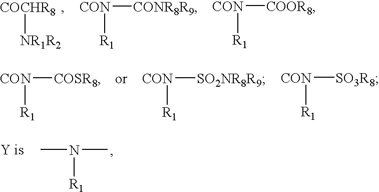

—O—,

—S(O)₀, 1 or 2,

—CH₂₋,

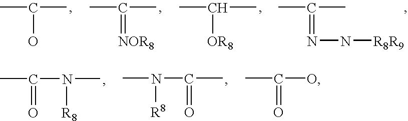

—CH₂—O—,

—O—CH₂—,

—CH₂S(O)₀, 1 or 2,

—S(O)_{0, 1 or 2-}CH₂—,

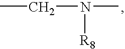

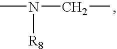

—CH═N, or

—N═CH—;

R₈ and R₉ independently are

hydrogen

C₁-C₂₀ alkyl or substituted C₁₋C₂₀ alkyl,

(CH₂)₀₋₆-aryl,

(CH₂)₀₋₆-heteroaryl, or

(CH₂)₀₋₆-cycloalkyl;

W, W¹, Z, and Z¹ independently are CR₁ or N;

and the pharmaceutically acceptable salts, isomers, stereoisomers, and solvates thereof.

Specific examples of compounds to be employed in the present method include:

(S)-4-Dibenzofuran-2-yl4-oxo-2-(2,2,2-trifluoroacetylamino)-butyric acid;

(R)-4-Dibenzofuran-2-yloxo-2-(2,2,2-trifluoroacetylamino)-butyric acid;

(S)-2-Aminodibenzofuran-2-yl4-oxo-butyric acid

(S)-2-Acetylamino-4-dibenzofuran-2-yl-4-oxo-butyric;

(S)-4-Dibenzofuran-2-yl-2-[3-(2,6-diisopropyl-phenyl)-ureido]-4-oxo-butyric acid;

(S)-2-BenzoylaminoA-4-dibenzofuran-2-yl-4-oxo-butyric acid

(S)-4-Dibenzofuran-2-yl-4-oxo-2-phenylacetylamino-butyric acid;

(S)-4-Dibenzofuran-2-yl-4-oxo-2-(3-phenyl-propionylamino)-butyric acid;

(S)-4-Dibenzofuran-2-yl-4-oxo-2-(7-phenyl-heptanoylamino)-butyric acid;

(S)-2-[(Biphenyl-4-carbonyl)-amino]-4-dibenzofuran-2-yl-4-oxo-butyric acid;

(S)-4-Dibenzofuran-2-yl-4-oxo-2-(dodecanoylamino)-butyric acid;

(S)-4-Dibenzofuran-2-yl-4-oxo-2-(dodecanoyl-amino)-butyric acid;

(S)-4-Dibenzofuran-2-yl-4-oxo-2-(2,2,2-trifluoroacetylamino)-butyric acid;

(R)-4-Dibenzofuran-2-yl-4-oxo-2-(2,2,2-trifluoroacetylamino)-butyric acid;

(S)-2-Amino-4-dibenzofuran-2-yl-4-oxo-butyric acid;

(S)-2-Acetylamino-4dibenzofuran-2-yl-4-oxo-butyric acid;

(S)-4-Dibenzofuran-2-yl-2-[3-(2,6-diisopropyl-phenyl)-ureido]-b 4-oxo-butyric acid;

(S)-2-Benzoylamino-4-dibenzofuran-2-yl-4-oxo-butyric acid;

(S)-4-Dibenzofuran-2-yl-4-oxo-2-phenylacetylaminobutyric acid;

(S)-4-Dibenzofuran-2-yl-4-oxo-2-(3-phenyl-propionylamino)-butyric acid;

(S)-4-Dibenzofuran-2-yl-4-oxo-2-(7-phenyl-heptanoylamino)-butyric acid;

(S)-2-[(Biphenyl-4 -carbonyl)-amino]-4-dibenzofuran-2-yl4-oxo-butyric acid;

(S)-4-Dibenzofuran-2-yl-4-oxo-2-(octanoylamino)-butyric acid; and

(S)-4-Dibenzofuran-2-yl-4-oxo-2-(dodecanoylamino)-butyric acid.

Tricyclic sulfonamide matrix metalloproteinase inhibitors include compounds of the formula

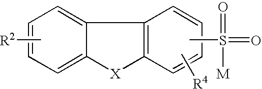

wherein M is a natural (L) alpha amino acid derivative having the structure

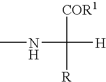

X is O, S, S(O)ₙ, CH₂, CO, or NH;

R is a side chain of a natural alpha amino acid;

R¹ is C₁-C₅ alkoxy, hydroxy, or —NHOR⁵;

R² and R⁴ are independently hydrogen, -C₁-C₅ alkyl, —NO₂, halogen, —OR⁵, —CN, —CO₂R⁵, —SO₃R⁵, —CHO, —COR⁵, —CONR⁵R⁶, —(CH₂)ₙNR⁵R⁶, —CF₃, or —NHCOR⁵;

each R⁵ and R⁶ are independently hydrogen or C₁-C₅ alkyl; and n is 0 to 2, and the pharmaceutically acceptable salts, ester, amides, and prodrugs thereof.

Specific compounds from this class to be employed include:

(L)-2-(Dibenzofuran-2-sulfonylamino)-4-methyl-pentanoic acid;

(L)-2-(Dibenzofuran-2-sulfonylamino)3-methyl-pentanoic acid

(L)-2-(Dibenzofuran-2-sulfonylamino)-3-phenyl-propionic acid;

(L)-2-(Dibenzofuran-2-sulfonylamino)-propionic acid;

(L)-2-(Dibenzofuran-2-sulfonylamino)3-methyl-butyric acid;

(Dibenzofuran-2-sulfonylamino)-acetic acid;

(L)-2-(Dibenzofuran-2-sulfonylamino)succinic acid;

(L)-2-(Dibenzofuran-2-sulfonylamino)-3-tritylsulfanyl-propionic acid;

(L)-2-(Dibenzofuran-2-sulfonylamino)-3-mercapto-propionic acid;

(L)-2-(Dibenzofuran-2-sulfonylamino)-3-methyl-pentanoic acid hydroxyamide;

(L)-2-(Dibenzofuran-2-sulfonylamino)-methyl-pentanoic acid;

(L)-2-(Dibenzofuran-2-sulfonylamino)-3-methyl-pentanoic acid;

(L)-2-(Dibenzofuran-2-sulfonylamino)-3-phenyl-propionic acid;

(L)-2-(Dibenzofuran-2-sulfonylamino)-propionic acid;

(L)-2-(Dibenzofuran-2-sulfonylamino)-3-methyl-butyric acid;

(Dibenzofuran-2-sulfonylamino)acetic acid;

(L)-2-(Dibenzofuran-2-sulfonylamino)-succinic acid;

(L)-2-(Dibenzofuran-2-sulfonylamino)-3-tritylsulfanyl-propionic acid;

(L)-2-(Dibenzofuran-2-sulfonylamino)-3-mercapto-propionic acid; and

(L)-2-(Dibenzofuran-2-sulfonylamino)-3-methyl-pentanoic acid hydroxyamide.

Additional tricyclic sulfonamides are defined by the formula:

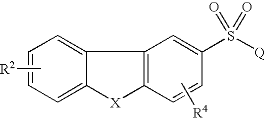

wherein Q is an unnatural amino acid;

X is O, S, S(O)ₙ, CH₂, CO, or NH;

R² and R⁴ are independently hydrogen, C₁-C₅ alkyl, —NO₂, halogen, —OR⁵, —CN, —CO₂R⁵, —SO₃R⁵, —CHO, —COR⁵, —CONR⁵R⁶, —(CH₂)ₙNR⁵R⁶, —CF₃, or —NHCOR⁵;

each R⁵ and R⁶ are independently hydrogen or C₁ -C₅ alkyl; and

n is 0 to 2, and the pharmaceutically acceptable salts, esters, amides, and prodrugs thereof.

Specific examples of such compounds include:

(S2-(Dibenzofuran-2-sulfonylamino)-4-phenyl-butyric acid;

2 (S)-3-[(Dibenzofuran-2-sulfonylamino)-methyl]-5-methyl-hexanoic acid;

(S)-2-(Dibenzofuran-2-sulfonylamino)4-phenyl-butyric acid; and

2 (S)-3-[(Dibenzofuran-2-sulfonylamino)methyl]-5-methyl-hexanoic acid.

Another general class of matrix metalloproteinase inhibitors, which are useful to combine with statins to treat and prevent heart failure and ventricular dilatation, are biphenyl butyric acid derivatives, including compounds of the formula:

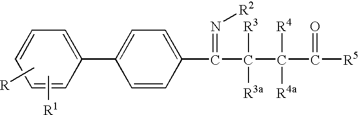

wherein R and R¹ are the same or different and are

hydrogen,

alkyl,

halogen,

nitro,

cyano,

trifluoromethyl,

—OR⁶ wherein R⁶ is hydrogen,

alkyl,

aryl,

arylalkyl,

heteroaryl, or

cycloalkyl,

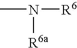

wherein R⁶ and R^{6a} are the same or different and are as defined above for R⁶,

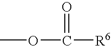

wherein R⁶ is as defined above,

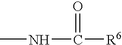

wherein R⁶ is as defined above,

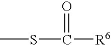

wherein R⁶ is as defined above, —SR⁶ wherein R⁶ as defined above,

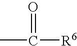

wherein R⁶ is as defined above, —CH₂-OR⁶ wherein R⁶ is as defined above,

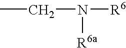

wherein R⁶ and R^{6a} are the same or different and are as defined above for R⁶,

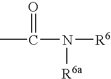

wherein R⁶ and R^{6a} are the same or different and are as defined above for R⁶,

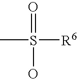

wherein R⁶ is as defined above,

cycloalkyl, or

heteroaryl, with the proviso that R and R¹ are not both hydrogen; R² is —OR⁶ wherein R⁶ is as defined above, or

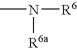

wherein R⁶ and R^{6a} are the same or different and are as defined above for R⁶;

R³, R^{3a}, R⁴, and R^{4a} are the same or different and are

hydrogen,

fluorine,

alkyl,

—(CH₂)ₙ-aryl wherein n is an integer from 1 to 6,

—(CH₂)ₙ-heteroaryl wherein n is as defined above,

—(CH₂)ₙ-cycloalkyl wherein n is as defined above,

—(CH₂)ₚ-X-(CH₂)_{q}-aryl wherein X is O, S, SO, SO₂, or NH, and p and q are each zero or an integer of 1 to 6, and the sum of p+q is not greater than six,

—(CH₂)ₚ-X-(CH₂)_{q}-heteroaryl wherein X, p, and q are as defined above, or

—(CH₂)ₙ-R⁷ wherein R⁷ is

N-phthalimido,

N-2,3-naphthyimido,

—OR⁶ wherein R⁶ is as defined above,

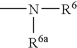

wherein R⁶ and R^{6a} are the same or different and are as defined above for R⁶,

—SR⁶ where R⁶ is as defined above,

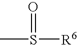

wherein R⁶ is as defined above,

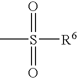

wherein R⁶ is as defined above,

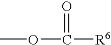

wherein R⁶ is as defined above,

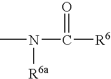

wherein R⁶ and R^{6a} are the same or different and are as defined above for R⁶,

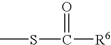

wherein R⁶ is as defined above,

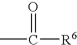

wherein R⁶ is as defined above,

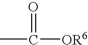

wherein R⁶ is as defined above, or

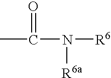

wherein R⁶ and R^{6a} are the same or different and are as defined above for R⁶, and

n is as defined above;

R⁵ is OH or SH; with the proviso that R³, R^{3a} , R⁴, and R^{4a} are hydrogen or at least one of R³, R^{3a}, R⁴, or R^{4a} is fluorine; and corresponding isomers thereof; or a pharmaceutically acceptable salt thereof.

Typical compounds from this class that are routinely utilized in combination with a statin to treat and prevent heart failure include:

4-(4-Chloro-biphenyl-4-yl)-4-hydroxyimino-butyric acid;

4-(4′-Bromo-biphenyl-4-yl)-4-hydroxyimino-butyric acid;

4-(4′-Chloro-biphenyl)-4-(dimethylhydrazono)-butyric acid;

4-(4′-Fluoro-biphenyl)-4-yl)-4-hydroxyimino-butyric acid;

(±)-4-(4′-Chloro-biphenyl-4-yl)-4-hydroxy-butyric acid;

4-(4′-Bromo-2′-fluoro-biphenyl)-4-yl)-4-hydroxyimino-butyric acid;

(±)-4-(4′-Chloro-biphenyl-4-yl)-3-fluoro-4-oxo-butyric acid;

4-(2′,4′-Dichloro-biphenyl-4-yl)-4-hydroxyimino-butyric acid;

4-(2′,4′-Difluoro-biphenyl-4-yl)-4-hydroxyimino-butyric acid;

(±)-4-(4′-Chloro-biphenyl-4-yl)-4-hydroxyimino-2-fluoro-2-(3-phenylpropyl)-butyric acid;

(±)-4-(4′-Chloro-biphenyl-4-yl)-4-hydroxyimino-2-fluoro-2-(2-phenylethyl)-butyric acid;

(±)-4-(4′-Chloro-biphenyl-4-yl)-4-hydroxyimino-2-fluoro-2-(3-phthalimidopropyl)-butyric acid;

(±)-4-(4′-Chloro-biphenyl-4-yl)-4-hydroxyimino-2-fluoro-2-(phenylthiomethyl)-butyric acid;

4-(4′-Chloro-2′-fluoro-biphenyl-4-yl)-4-hydroxyimino-butyric acid;

4-Hydroxyimino-4-(4′-trifluoromethyl-biphenyl-4-yl)-butyric acid;

4-(4′-Chloro-biphenyl-4-yl)-4-methoxyimino-butyric acid;

(±)-4-(4′Chloro-biphenyl-4-yl)-2-fluoro-2-[2-(1,3-dioxo-1,3-dihydroisoindol-2-yl)-ethyl]-4-hydroxyimino-butyric acid;

(±)-4-(4′-Chloro-biphenyl-4-yl)-4-hydroxyimino-2-fluoro-2-(1H-indol-3-yl)methyl-butyric acid;

(±)-4-(4′-Chloro-biphenyl-4-yl)-4-hydroxyimino-2-fluoro-2-methylbutyric acid;

(±)-2-[2-(4′-Chloro-biphenyl-1-yl)-2-hydroxyiminoethyl]-2-fluoro-6-phenyl-hexanoic acid;

(±)-4-(4′-Chloro-biphenyl-4-yl)-2-fluoro-2-[2-(1,3-dioxo-1,3-dihydrobenzo[F]isoindol-2-yl)-ethyl]-4-hydroxyimino-butyric acid;

(±)-2-[2-(4′-Chloro-biphenyl-4-yl)-2-hydroxyiminoethyl]-6-(1,3-dioxo-1,3-dihydro-isoindol-2-yl)-2-fluoro-hexanoic acid;

(±)-4-(4′-Chloro-biphenyl-4-yl)-4-hydroxyimino-2-fluoro-2-[2-(phenylethylcarbamoyl)-ethyl]-butyric acid;

4-(4′-Chloro-biphenyl-4-yl)-3,3-difluoro-4-hydroxyimino-butyric acid;

(±)-4-(4′-Chloro-biphenyl-4-yl)-3,3-dimethyl-2-fluoro-4-hydroxyimino-butyric acid;

(±)-4-(4′-Chloro-biphenyl-4-yl)-2,2-dimethyl-3-fluoro-4-hydroxyimino-butyric acid;

4-(4′-Chloro-biphenyl-4-yl)-2,2-difluoro-4-hydroxyimino-butyric acid; and

4-(4′-Chloro-biphenyl-4-yl)-2,2,3,3-tetrafluoro-4-hydroxyimino-butyric acid.

A compound selected from the group consisting of:

4-(4′-Chloro-biphenyl-4-yl)-4-hydroxyimino-butyric acid;

4-(4′-Bromo-biphenyl-4-yl)-4-hydroxyimino-butyric acid;

4-(4′-Chloro-biphenyl4-yl)-4-(dimethylhydrazono)-butyric acid;

4-(4′-Fluoro-biphenyl-4-yl)-4-hydroxyimino-butyric acid;

(±)-4-(4′-Chloro-biphenyl-4-yl)-4-hydroxy-butyric acid; 4-(4′-Bromo-2′-fluoro-biphenyl-4-yl)-4-hydroxyimino-butyric acid;

(±)-4-(4′-Chloro-biphenyl-4-yl)-3-fluoro-4-oxo-butyric acid;

4-(2′,4′-Dichloro-biphenyl-4-yl)-4-hydroxyimino-butyric acid;

4-(2′,4′-Difluoro-biphenyl-4-yl)-4-hydroxyimino-butyric acid;

(±)-4-(4′-Chloro-biphenyl-4-yl)-4-hydroxyimino-2-fluoro-2-(3-phenylpropyl)-butyric acid;

(±)-4-(4′-Chloro-biphenyl-4-yl)-4-hydroxyimino-2-fluoro-2-(2-phenylethyl)-butyric acid;

(±)-4-(4′-Chloro-biphenyl-4-yl)-4-hydroxyimino-2-fluoro-2-(3-phthalimidopropyl)-butyric acid;

(±)-4-(4′-Chloro-biphenyl-4-yl)-4-hydroxyimino-2-fluoro-2-(phenylthiomethyl)-butyric acid;

4-(4′-Chloro-2′-fluoro-biphenyl-4-yl)-4-hydroxyimino-butyric acid;

4-hydroxyimino-4-(4′-trifluoromethyl-biphenyl-4-yl)-butyric acid;

4-(4′-Chloro-biphenyl-4-yl)-4-methoxyimino-butyric acid;

(±)-4-(4′-Chloro-biphenyl-4-yl)-2-fluoro-2-[2-(1,3-dioxo-1,3-dihydroisoindol-2-yl)-ethyl]-4-hydroxyimino-butyric acid;

(±)-4-(4′-Chloro-biphenyl-4-yl)-4-hydroxyimino-2-fluoro-2-(1H-indol-3-yl)methyl-butyric acid;

(±)-4-(4′-Chloro-biphenyl-4-yl)-4-hydroxyimino-2-fluoro-2-methyl-butyric acid;

(±)-2-[2-(4′-Chloro-biphenyl-4-yl)-2-hydroxyiminoethyl]-2-fluoro-6-phenyl-hexanoic acid;

(±)-4-(4′-Chloro-biphenyl-4-yl)-2-fluoro-2-[2-(1,3-dioxo-1,3-dihydro-benzo[F]isoindol-2-yl)-ethyl]-4-hydroxyimino-butyric acid;

(±)-2-[2-(4′-Chloro-biphenyl-4-yl)-2-hydroxyiminoethyl]-6-(1,3-dioxo-1,3-dihydro-isoindol-2-yl)-2-fluoro-hexanoic acid;

(±)-4-(4′-Chloro-biphenyl-4-yl)-4-hydroxyimino-2-fluoro-2-[2-(phenylethylcarbamoyl)-ethyl]-butyric acid;

4-(4′-Chloro-biphenyl-4-yl)-3,3-difluoro-4-hydroxyimino-butyric acid;

(±)-4-(4′-Chloro-biphenyl-4-yl)-3,3-dimethyl-2-fluoro4-hydroxyimino-butyric acid;

(±)-4-(4′-Chloro-biphenyl-4-yl)-2,2-dimethyl-3-fluoro4-hydroxyimino-butyric acid;

4-(4′-Chloro-biphenyl-4-yl)-2,2-difluoro-4-hydroxyimino-butyric acid; and

4-(4′-Chloro-biphenyl-4-yl)-2,2,3,3-tetrafluoro-4-hydroxyimino-butyric acid.

Biphenyl sulfonamides are also particularly good in the present method. Such compounds include those of the formula:

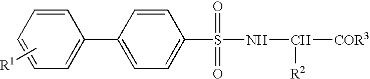

wherein:

R¹ is C₁ -C₆ alkyl, halo, nitro, NR⁴R⁵, cyano, OR⁴, and COOR⁴;

R² is C₁ -C₆ alkyl, optionally substituted by phenyl, substituted phenyl, NR⁴R⁵, OR⁶,

carboxy, carboxamido,

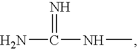

thio, methylthio, indole, imidazole, phthalimido, phenyl, and substituted phenyl;

R³ is OH, OC₁-C₆ alkyl, or NHOH;

R⁴ is hydrogen, C₁-C₆ alkyl, or C₁-C₆ alkanoyl;

R⁵ is hydrogen or C₁-C₆ alkyl; and

R⁶ is hydrogen, C₁-C₆ alkyl, C₁-C₆ alkanoyl, phenyl, or substituted phenyl.

Specific compounds which can be employed include a compound of the above formula wherein R¹ is at the 4′ position.

Another class of matrix metalloproteinase inhibitors useful in the present method are the heterocyclic substituted phenyl butyric acid derivatives, for example those defined by the formula:

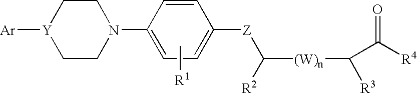

Ar is selected from phenyl,

phenyl substituted with

alkyl,

NO₂,

halogen,

OR⁵ wherein R⁵ is hydrogen or alkyl,

CN,

CO₂R⁵ wherein R⁵ is as defined above,

SO₃R⁵ wherein R⁵ is as defined above,

CHO,

COR⁵ wherein R⁵ is as defined above,

CONHR⁵ wherein R⁵ is as defined above, or

NHCOR⁵ wherein R⁵ is as defined above,

2-naphthyl, or

heteroaryl;

R¹ is selected from hydrogen,

methyl,

ethyl,

NO₂,

halogen,

OR⁵ wherein R⁵ is as defined above,

CN,

CO₂R⁵ wherein R⁵ is as defined above,

SO₃R⁵ wherein R⁵ is as defined above,

CHO, or

COR⁵ wherein R⁵ is as defined above;

R² and R³ are the same or different and independently selected from hydrogen,

alkyl,

—(CH₂)ᵥ-aryl wherein v is an integer from 1 to 5,

—(CH₂)ᵥ-heteroaryl wherein v is as defined above,

—(CH₂)ᵥ-cycloalkyl wherein v is as defined above,

—(CH₂)ₚ-X—(CH₂)_{q}-aryl wherein X is O or S and p and q is each zero or an integer of 1 to 5, and the sum of p+q is not greater than an integer of 5,

—(CH₂)ₚ-X—(CH₂)_{q}-heteroaryl wherein X, p, and q are as defined above,

—(CH₂)ₜNR⁶R^{6a}, wherein t is zero or an integer of from 1 to 9 and R⁶ and R^{6a} are each the same or different and are as defined above for R⁵,

—(CH₂)ᵥSR⁵, wherein v and R⁵ are as defined above,

—(CH₂)ᵥCO₂R⁵, wherein v and R⁵ are as defined above, or

—(CH₂)ᵥCONR⁶R^{6a}, wherein R⁶ and R^{6a} are the same or different and are as defined above for R⁵ and v is as defined above;

R³ is additionally —(CH₂)ᵣR⁷ wherein r is an integer from 1 to 5 and R⁷ is 1,3-dihydro-1,3-dioxo-2H-isoindol-2-yl, or 1,3,-dihydro-1,3-dioxobenzo[f]isoindol-2-yl;

Y is CH or N;

Z is

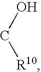

wherein R¹⁰ is as defined above for R² and R³, and is independently the same or different from R² and R³ provided that

when Z is

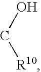

then R⁴ must be OH,

C═O,

C═NOR⁵ wherein R⁵ is as defined above, or

C═N-NR⁶R^{6a} wherein R⁶ and R^{6a} are the same or different and are as defined above for R⁵;

W is —CHR⁵ wherein R⁵ is as defined above;

n is zero or an integer of 1;

R⁴ is OH,

NR⁶R^{6a} wherein R⁶ and R^{6a} are the same or different and are as defined above for R⁵, when R⁴ is NR⁶R^{6a} then Z must be C═O or

NHOR⁹ wherein R⁹ is hydrogen, alkyl, or benzyl; and corresponding isomers thereof; or a pharmaceutically acceptable salt thereof.

Especially preferred MMP inhibitors have the formula

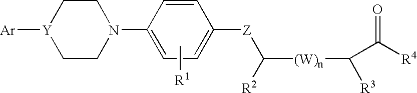

Ar is selected from phenyl,

phenyl substituted with

alkyl,

NO₂,

halogen,

OR⁵ wherein R⁵ is hydrogen or alkyl,

CN,

CO₂R⁵ wherein R⁵ is as defined above,

SO₃R⁵ wherein R⁵ is as defined above,

CHO,

COR⁵ wherein R⁵ is as defined above,

CONHR⁵ wherein R⁵ is as defined above, or

NHCOR⁵ wherein R⁵ is as defined above,

2-naphthyl, or

heteroaryl;

R¹ is selected from hydrogen,

methyl,

ethyl,

NO₂;

halogen,

OR⁵ wherein R⁵ is as defined above,

CN,

CO₂R⁵ wherein R⁵ is as defined above,

SO₃R⁵ wherein R⁵ is as defined above,

CHO, or

COR⁵ wherein R⁵ is as defined above;

R² and R³ are the same or different and independently selected from hydrogen,

alkyl,

—(CH₂)ᵥ-aryl wherein v is an integer from 1 to 5,

—(CH₂)ᵥ-heteroaryl wherein v is as defined above,

—(CH₂)ᵥ-cycloalkyl wherein v is as defined above,

—(CH₂)ₚ-X—(CH₂)_{q}-aryl wherein X is O or S and p and q is each zero or an integer of 1 to 5, and the sum of p+q is not greater than an integer of 5,

—(CH₂)ₚ-X—(CH₂)_{q}-heteroaryl wherein X, p, and q are as defined above,

—(CH₂)ₜNR⁶R^{6a} , wherein t is zero or an integer of from 1 to 9 and R⁶ and R^{6a} are each the same or different and are as defined above for R⁵,

—(CH₂)ᵥSR⁵, wherein v and R⁵ are as defined above,

—(CH₂)ᵥCO₂R⁵, wherein v and R⁵ are as defined above, or

—(CH₂)ᵥCONR⁶R^{6a} , wherein R⁶ and R^{6a} are the same or different and are as defined above for R⁵ and v is as defined above;

R³ is additionally —(CH₂)ᵣR⁷ wherein r is an integer from 1 to 5 and R⁷ is 1,3-dihydro-1,3-dioxo-2H-isoindol-2-yl, or 1,3,-dihydro-1,3-dioxo-benzo[f]isoindol-2-yl;

Y is CH or N;

z is

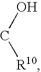

wherein R¹⁰ is as defined above for R² and R³, and is independently the same or different from R² and R³ provided that

when Z is

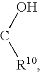

then R⁴ must be OH,

C═O,

C═NOR⁵ wherein R⁵ is as defined above, or

C═N-NR⁶R^{6a} wherein R⁶ and R^{6a} are the same or different and are as defined above for R⁵;

W is —CHR5 wherein R⁵ is as defined above;

n is zero or an integer of 1;

R⁴ is OH,

NR⁶R^{6a} wherein R⁶ and R^{6a} are the same or different and are as defined above for R⁵, when R⁴ is NR⁶R^{6a} then Z must be C═O or

NHOR⁹ wherein R⁹ is hydrogen, alkyl, or benzyl; and corresponding isomers thereof; or a pharmaceutically acceptable salt thereof.

Preferred compounds to be employed include:

4Oxo-4-[4-(4-phenyl-piperidin-1-yl)-phenyl]-butyric acid;

4Oxo-4-[4-(4phenyl-piperidin-1-yl)-phenyl]-butyric acid, potassium salt;

N-Hydroxy4-oxo-4-[4-(4-phenyl-piperidin-1-yl)-phenyl]-butyramide;

E/Z-4-Hydroxyimino-4-[4-(4-phenyl-piperidin-1-yl)-phenyl]-butyric acid;

E/Z-4-Benzyloxyimino-4-[4-(4-phenyl-piperidin-1-yl)-phenyl]-butyric acid;

4-Oxo-4-[4-(4-phenyl-piperazin-1-yl)-phenyl]-butyric acid;

(±)3-Methyl-5-oxo-5-[4-(4-phenyl-piperidin-1-yl)-phenyl]-pentanoic acid;

4-Oxo-4-[4-(4-phenyl-piperidin-1-yl)-phenyl]-butyric acid;

4-Oxo-4-[4-(4phenyl-piperidin-1-yl)-phenyl]-butyric acid, potassium salt;

N-Hydroxy-4-oxo-4-[4-(4-phenyl-piperidin-1-yl)-phenyl]-butyramide;

E/Z-4-Hydroxyimino-4-[4-(4phenyl-piperidin-1-yl)-phenyl]-butyric acid;

E/Z-4-Benzyloxyimino-4-[4-(4-phenyl-piperidin-1-yl)-phenyl]-butyric acid;

4-OxoA-4-[4-(4-phenyl-piperazin-1-yl)-phenyl]-butyric acid; and

(±)3-Methyl-5-oxo-5-[4-(4-phenyl-piperidin-1-yl)-phenyl]-pentanoic acid.

A compound which is 4-oxo-4-[4-(4 -phenyl-piperidin-1-yl)-phenyl]-butyric acid.

Similar compounds, which are sulfonamide derivatives, have the formula:

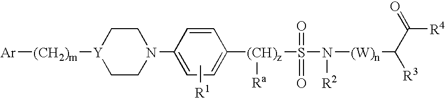

wherein:

Ar is selected from phenyl;

phenyl substituted with alkyl, —NO₂, halogen, —OR⁵, —CN, —CO₂R⁵, —SO₃R⁵, —CHO, —COR⁵, —CONHR⁵, —NHR⁵, or —NHCOR⁵;

heteroaryl; or

2-naphthyl;

R¹ is hydrogen, methyl, —NO2, —Cl, —NH₂, —NHCO₂CH₃, —OH, or —CO₂H;

R² and R³ are the same or different and are independently selected from hydrogen, alkyl, —(CH₂)ᵥ-aryl, —(CH₂)ᵥ-heteroaryl, —(CH₂)ᵥ-cycloalkyl, —(CH₂)ₚ-X—(CH₂)_{q}-aryl, —(CH₂)ₚ-X—(CH₂)_{q}-heteroaryl, —(CH₂)ₜNR⁶R^{6a}, —(CH₂)ᵥR⁷, —(CH₂)ᵥCO₂R⁵, —(CH₂)ᵥCONR⁶R6a, or —(CH₂)_{V}SR⁵;

m is zero or 1;

Y is CH or N; provided that when m=1, Y does not=N;

z is zero or 1;

W is —CHR⁸;

n is zero or 1;

R⁴ is —OH, —NR⁶R^{6a}, or —NHOR⁹;

R⁵ is hydrogen or alkyl;

v is 1 to 5;

X is O or S;

p and q are independently 1 to 5, provided that p+q is not greater than 5; t is 1 to 9;

R⁶ and R^{6a} are each the same or different and are hydrogen or alkyl;

R⁷ is 1,3dihydro-1,3-dioxo-2H-isoindol-2-yl, or 1,3-dihydro-1,3-dioxobenzo[f]isoindol-2-yl;

R⁸ is hydrogen or alkyl; and

R⁹ is hydrogen, alkyl, or benzyl; or

a pharmaceutically acceptable salt thereof.

Specific sulfonamide derivatives to be employed in the present method include:

[4-(4-Phenyl-piperidin-1-yl)-benzenesulfonylamino]-acetic acid;

N-Hydroxy-2-[4-(4phenyl-piperidin-1-yl)-benzenesulfonylamino]-acetamide;

3-[4-(4-Phenyl-piperidin-1-yl)-benzenesulfonylamino)-propionic acid;

(R)-4-Methyl-2-[4-(4-phenyl-piperidin-1-yl)-benzenesulfonylamino]-pentanoic acid;

(S)-4-Methyl-2-[4-(4-phenyl-piperidin-1-yl)-benzenesulfonylamino]-pentanoic acid;

(S)-3-Phenyl-2-[4-(4-phenyl-piperidin-1-yl)-benzenesulfonylamino]-propionic acid;

(R)-3-Phenyl-2-[4-(4-phenyl-piperidin-1-yl)-benzenesulfonylamino]-propionic acid;

(S)-3-(1H-Indol-3-yl)-2-[4-(4-phenyl-piperidin-1-yl)-benzenesulfonylamino]-propionic acid;

(±)-5-Phenyl-2-[4-(4-phenyl-piperidin-1-yl)-benzenesulfonylamino]-pentanoic acid;

[4-(4-Phenyl-piperazin-1-yl)-benzene-sulfonylamino]-acetic acid;

{Isobutyl-[4-(4-phenyl-piperidin-1-yl)-benzenesulfonyl]amino}-acetic acid;

(S)-4-Phenyl-2-[4-(4-phenyl-piperidin-1-yl)-benzenesulfonylamino]-butyric acid;

(R)-2-[4-(4Phenyl-piperidin-1-yl)-benzenesulfonylamino]-3-tritylsulfanyl-propionic acid, sodium salt;

(R)-3-(1H-Indol-3-yl)-2-[4-(4-phenyl-piperidin-1-yl)-benzenesulfonylamino]-propionic acid, disodium salt, monohydrate;

(S)-2-{4-[-4-(4-Hydroxy-phenyl)-piperazin-1-yl]-benzenesulfonylamino}-3-phenyl-propionic acid;

(S)-2-{4-[-4-(4-Chloro-phenyl)-piperazin-1-yl]-benzenesulfonylamino}-3-phenyl-propionic acid, hydrochloride;

(R)-3-Mercapto-2-[4-(4-phenyl-piperidin-1-yl)-benzenesulfonylamino]-propionic acid, trifluoracetic acid salt;

(S)-2-[4-(4-Benzyl-piperidin-1-yl)-benzenesulfonylamino]-3-phenyl-propionic acid;

(S)-3-(4-Benzyloxy-phenyl)-2-[4-(4phenyl-piperidin-1-yl)-benzenesulfonylamino]-propionic acid;

(S)-3-(4-Hydroxy-phenyl)-2-[4-(4phenyl-piperidin-1-yl)-benzenesulfonylamino]-propionic acid;

(S)-3-Phenyl-2-[4-(4-phenyl-piperazin-1-yl)-benzenesulfonylamino]-propionic acid;

(S)-2-{4-[-4-(3-Methoxy-phenyl)-piperazin-1-yl]-benzenesulfonylamino)-3-phenyl-propionic acid;

(S)-2-{4-[-4-(3-Hydroxy-phenyl)-piperazin-1-yl]-benzenesulfonylamnino}-3-phenyl-propionic acid hydrobromide;

(S)-2-{4-[-4-(4-Methoxy-phenyl)-piperazin-1-yl]-benzenesulfonylamino}-3-phenyl-propionic acid;

(R)-4-Methyl-2-[4-(4-phenyl-piperidin-1-yl)-benzenesulfonylamino]-pentanoic acid;

(S)-4-Methyl-2-[4-(4-phenyl-piperidin-1-yl)-benzenesulfonylamino]-pentanoic acid;

(S)-3-Phenyl-2-[4-(4-phenyl-piperidin-1-yl)-benzenesulfonylamino]-propionic acid;

(R)-3-Phenyl-2-[4-(4-phenyl-piperidin-1-yl)-benzenesulfonylamino]-propionic acid;

(S)-3-(1H-Indol-3-yl)-2-[4-(4-phenyl-piperidin-1-yl)-benzenesulfonylamino]-propionic acid;

[4-(4--Phenyl-piperidin-1-yl)-benzenesulfonylamino]-acetic acid;

N-Hydroxy-2-[4-(4-phenyl-piperidin-1-yl)-benzenesulfonylamino]-acetamide;

3-[4-(4-Phenyl-piperidin-1-yl)-benzenesulfonylamino]-propionic acid;

(R)-4-Methyl-2-[4-(4-phenyl-piperidin-1-yl)-benzenesulfonylamino]-pentanoic acid;

(S)-4-Methyl-2-[4-(4-phenyl-piperidin-1-yl)-benzenesulfonylamino]-pentanoic acid;

(S)-3-Phenyl-2-[4-(4-phenyl-piperidin-1-yl)-benzenesulfonylamino]-propionic acid;

(R)-3-Phenyl-2-[4-(4-phenyl-piperidin-1-yl)-benzenesulfonylamino]-propionic acid;

(S)-3-(1H-Indol-3-yl)-2-[4-(4-phenyl-piperidin-1-yl)-benzenesulfonylamino]-propionic acid;

(±)-5-Phenyl-2-[4-(4-phenyl-piperidin-1-yl)-benzenesulfonylamino]-pentanoic acid;

[4-(4-Phenyl-piperazin-1-yl)-benzene-sulfonylamino]-acetic acid;

{Isobutyl-[4-(4-phenyl-piperidin-1-yl)-benzenesulfonyl]amino}-acetic acid;

(S)-4-Phenyl-2-[4-(4phenyl-piperidin-1-yl)-benzenesulfonylamino]-butyric acid;

(R)-2-[4-(4-Phenyl-piperidin-1-yl)-benzenesulfonylamino]-3-tritylsulfanyl-propionic acid, sodium salt;

(R)-3-(1H-Indol-3-yl)-2-[4-(4-phenyl-piperidin-1-yl)-benzenesulfonylamino]-propionic acid, disodium salt, monohydrate;

(S)-2-{4-[-4-(4-Hydroxy-phenyl)-piperazin-1-yl]-benzenesulfonylamino)-3-phenyl-propionic acid;

(S)-2-{4-[-4-(4-Chloro-phenyl)-piperazin-1-yl]-benzenesulfonylamino}-3-phenyl-propionic acid, hydrochloride;

(R)-3-Mercapto-2-[4-(4-phenyl-piperidin-1-yl)-benzenesulfonylamino]-propionic acid, trifluoracetic acid salt;

(S)-2-[4-(4-Benzyl-piperidin-1-yl)-benzenesulfonylamino]-3-phenyl-propionic acid;

(S)-3-(4-Benzyloxy-phenyl)-2-[4-(4-phenyl-piperidin-1-yl)-benzenesulfonylamino]-propionic acid;

(S)-3-(4-Hydroxy-phenyl)-2-[4-(4-phenyl-piperidin-1-yl)-benzenesulfonylamino]-propionic acid;

(S)-3-Phenyl-2-[4-(4phenyl-piperazin-1-yl)-benzenesulfonylamino]-propionic acid;

(S)-2-{4-[-4-(3-Methoxy-phenyl)-piperazin-1-yl]-benzenesulfonylamino)-3-phenyl-propionic acid;

(S)-2-{4-[-4-(3-Hydroxy-phenyl)-piperazin-1-yl]-benzenesulfonylamino)-3-phenyl-propionic acid hydrobromide;

(S)-2-{4-[-4-(4-Methoxy-phenyl)-piperazin-1-yl]-benzenesulfonylamino)-3-phenyl-propionic acid;

(R)-4-Methyl-2-[4-(4-phenyl-piperidin-1-yl)-benzenesulfonylamino]-pentanoic acid;

(S)-4-Methyl-2-[4-(4-phenyl-piperidin-1-yl)-benzenesulfonylamino]-pentanoic acid;

(S)-3-Phenyl-2-[4-(4-phenyl-piperidin-1-yl)-benzenesulfonylamino]-propionic acid;

(R)-3-Phenyl-2-[4-(4-phenyl-piperidin-1-yl)-benzenesulfonylamino]-propionic acid; and

(S)-3-(1H-Indol-3-yl)-2-[4-(4-phenyl-piperidin-1-yl)-benzenesulfonylamino]-propionic acid.

Additional specific compounds, which can be used, include:

2-(Dibenzofuran-2-sulfonylamino)-3-(4-fluoro-phenyl)-propionic acid;

2-(Dibenzofuran-2-sulfonylamino)-3-phenyl-propionic acid;

3-(4-tert-Butoxy-phenyl)-2-(dibenzofuran-2-sulfonylamino)-propionic acid;

(Dibenzofuran-2-sulfonylamino)-phenyl-acetic acid;

3-tert-Butoxy-2-(dibenzofuran-2-sulfonylamino)-propionic acid;

2-(Dibenzofuran-2-sulfonylamino)-3-(1H-imidazol-4-yl)-propionic acid;

2-(Dibenzofuran-2-sulfonylamino)-3-hydroxy-propionic acid;

3-Benzyloxy-2-(dibenzofuran-2-sulfonylamino)-propionic acid;

6-Benzyloxycarbonylamino-2-(dibenzofuran-2-sulfonylamino)-hexanoic acid;

5-Benzyloxycarbonylamino-2-(dibenzofuran-2-sulfonylamino)-pentanoic acid;

(Dibenzofuran-2-sulfonylamino)-(4methoxy-phenyl)-acetic acid;

3-Chloro-2-(dibenzofuran-2-sulfonylamino)-propionic acid;

3-(4-Benzyloxy-phenyl)-2-(dibenzofuran-2-sulfonylamino)-propionic acid;

2-(Dibenzofuran-2-sulfonylamino)-5-p-tolyl-sulfanylamino-pentanoic acid;

2-(Dibenzofuran-2-sulfonylamino)-4-mercapto-butyric acid;

3-(4-Bromo-phenyl)-2-(dibenzofuran-2-sulfonyl-amino)-propionic acid;

2-(Dibenzofuran-2-sulfonylamino)-butyric acid;

1-(Dibenzofuran-2-sulfonylamino)-cyclopropane-carboxylic acid;

3-(4-Chloro-phenyl)-2-(dibenzofuran-2-sulfonyl-amino)-propionic acid;

2-(Dibenzofuran-2-sulfonylamino)-3-(1H-indol-3-yl)-propionic acid;

2-(4′-Bromo-biphenyl-4-sulfonylamino)-6-(4-fluorobenzenesulfonylamino)-hexanoic-acid;

2-(4′-Bromo-biphenyl-4-sulfonylamino)-6-(4methoxybenzenesulfonylamino)-hexanoic acid;

6-(4-Bromo-benzenesulfonylamino)-2-(4′-bromo-biphenyl-4-sulfonylamino)-hexanoic-acid;

6-(2-Acetylamino-thiazole-5-sulfonylamino)-2-(4′-bromo-biphenyl-4-sulfonylamino)-hexanoic-acid;

6-(4-Acetylamino-benzenesulfonylamino)-2-(4′-bromo-biphenyl-4-sulfonylamino)-hexanoic-acid;

6-Benzenesulfonylamino-2-(4′-bromo-biphenyl-4-sulfonylamino)-hexanoic acid;

2-(4′-Bromo-biphenyl-4-sulfonylamino)-6-(pentane-1-sulfonylamino)-hexanoic acid;

2-(4′-Bromo-biphenyl-4-sulfonylamino)-6-(naphthalene-2-sulfonylamino)-hexanoic-acid;

2-(4′-Bromo-biphenyl-4-sulfonylamino)-6-(naphthalene-1-sulfonylamino)-hexanoic-acid;

2-(4′-Bromo-biphenyl-4-sulfonylamino)-6-(2-phenyl-ethenesulfonylamino)-hexanoic-acid;

2-(4′-Bromo-biphenyl-4-sulfonylamino)-6-phenyl-acetylaminohexanoic acid;

2-(4′-Bromo-biphenyl-4-sulfonylamino)-6-[2-(4-chloro-phenoxy)-cetylamino]-hexanoic acid;

2-(4′-Bromo-biphenyl-4-sulfonylamino)-6-[2-(4-chloro-phenoxy)-2-methyl-propionylamino]-hexanoic acid;

2-(4′-Bromo-biphenyl-4-sulfonylamino)-6-[2-(pyridin-4-ylsulfanyl)-acetylamino]-hexanoic acid;

2-(4′-Bromo-biphenyl-4-sulfonylamino)-6-[2-(2,4dichlorophenoxy)-acetylamino]-hexanoic acid;

2-(4′-Bromo-biphenyl-4-sulfonylamino)-6-(2-thiophen-2-yl-acetylamino)-hexanoic acid;

2-(4′-Bromo-biphenyl-4-sulfonylamino)-6-(3-phenyl-acryloylamino)-hexanoic acid;

2-(4′-Bromo-biphenyl-4-sulfonylamino)-6-(7-phenyl-heptanoylamino)-hexanoic acid;

2-(4′-Bromo-biphenyl-4-sulfonylamino)-6-[2-(2-trifluoromethyl-phenyl)-acetylamino]-hexanoic acid;

2-(4′-Bromo-biphenyl-4-sulfonylamino)-6-(2-phenoxy-butyrylamino)-hexanoic acid;

2-(4′-Bromo-biphenyl-4-sulfonylamino)-6-(2-phenyl-sulfanyl-acetylamino)-hexanoic acid;

2-(4′-Bromo-biphenyl-4-sulfonylamino)-6-(2-phenoxy-acetylamino)-hexanoic acid;

2-(4′-Bromo-biphenyl-4-sulfonylamino)-6-[2-(3,4-dimethoxy-phenyl)-acetylamino]-hexanoic acid;

2-(4′-Bromo-biphenyl-4-sulfonylamino)-6-[2-(4-tert-butyl-phenoxy)-acetylamino]-hexanoic acid;

2-(4′-Bromo-biphenyl-4-sulfonylamino)-6-[3-(3,4-dimethoxy-phenyl)-propionylamino]-hexanoic acid;

2-(4′-Bromo-biphenyl-4-sulfonylamino)-6-(2-cyclopent-1-enyl-acetylamino)-hexanoic acid;

2-(4′-Bromo-biphenyl-4-sulfonylamino)-6-[2-(4-methoxy-phenoxy)-acetylamino]-hexanoic acid;

2-(4′-Bromo-biphenyl-4-sulfonylamino)-6-[2-(naphthalen-1-yloxy)-acetylamino]-hexanoic acid;

2-(4′-Bromo-biphenyl-4-sulfonylamino)-6-[2-(4-nitro-phenoxy)-acetylamino]-hexanoic acid;

2-(4′-Bromo-biphenyl-4-sulfonylamino)-6-[4-(4-chloro-3-methyl-phenoxy)-butyrylamino]-hexanoic acid;

2-(4′-Bromo-biphenyl-4-sulfonylamino)-6-[3-(4-methoxy-phenyl)-propionylamino]-hexanoic acid;

2-(4′-Bromo-biphenyl-4-sulfonylamino)-6-(2-pyridin-3-yl-acetylamino-hexanoic acid;

6-(2-Benzo[1,3]dioxol-5-yl-acetylamino)-2-(4′-bromo-biphenyl-4-sulfonylamino)-hexanoic acid;

2-(4′-Bromo-biphenyl-4-sulfonylamino)-6-(2-pyridin-2-yl-acetylamino)-hexanoic acid;

2-(4′-Bromo-biphenyl-4-sulfonylamino)-6-[2-(4-tert-butyl-phenoxy)-acetylamino]-hexanoic acid;

2-(4′-Bromo-biphenyl-4-sulfonylamino)-6-[3-(3,4-dimethoxy-phenyl)-propionylamino]-hexanoic acid;

2-(4′-Bromo-biphenyl-4-sulfonylamino)-6-(2-cyclopent-1-enyl-acetylamino)-hexanoic acid;

2-(4′-Bromo-biphenyl-4-sulfonylamino)-6-[2-(4-methoxy-phenoxy)-acetylamino]-hexanoic acid;

2-(4′-Bromo-biphenyl-4-sulfonylamino)-6-[2-(naphthalen-1-yloxy)-acetylamino]-hexanoic acid;

2-(4′-Bromo-biphenyl-4-sulfonylamino)-6-[2-(4-nitro-phenoxy)-acetylamino]-hexanoic acid;

2-(4′-Bromo-biphenyl-4-sulfonylamino)-6-[4-(4chloro-3-methyl-phenoxy)-butyrylamino]-hexanoic acid;

2-(4′-Bromo-biphenyl-4-sulfonylamino)-6-[3-(4-methoxy-phenyl)-propionylamino]-hexanoic acid;

2-(4′-Bromo-biphenyl-4-sulfonylamino)-6-(2-pyridin-3-yl-acetylamino)-hexanoic acid;

6-(2-Benzo[1,3]dioxol-5-yl-acetylamino)-2-(4′-bromo-biphenyl-1-sulfonylamino)hexanoic acid;

2-(4′-Bromo-biphenyl-4-sulfonylamino)-6-(2-pyridin-2-yl-acetylamino)-hexanoic acid;

2-(4′-Bromo-biphenyl-4-sulfonylamino)-6-[4-(4-nitro-phenyl)-butyrylamino]-hexanoic acid;

2-(4′-Bromo-biphenyl-4-sulfonylamino)-6-[2-(4-tert-butyl-phenoxy)-acetylamino]-hexanoic acid;

2-(4′-Bromo-biphenyl-4-sulfonylamino)-6-[3-(3,4-dimethoxy-phenyl)-propionylamino]-hexanoic acid;

2-(4′-Bromo-biphenyl-4-sulfonylamino)-6-(2-cyclopent-1-enyl-acetylamino)-hexanoic acid;

2-(4′-Bromo-biphenyl-4-sulfonylamino)-6-[2-(4-methoxy-phenoxy)-acetylamino]-hexanoic acid;

2-(4′-Bromo-biphenyl-4-sulfonylamino)-6-(4-phenyl-butyrylamino)-hexanoic acid;

2-(4′-Bromo-biphenyl-4-sulfonylamino)-6-[4-(4-chloro-3-methyl-phenoxy)-butyrylamino]-hexanoic acid;

2-(4′-Bromo-biphenyl-4-sulfonylamino)-6-[3-(4-chloro-phenyl)-propionylamino]-hexanoic acid;

2-(4′-Bromo-biphenyl-4-sulfonylamino)-6-[3-(4-methoxy-phenyl)-propionylamino]-hexanoic acid;

2-(4′-Bromo-biphenyl-4-sulfonylamino)-6-(2-pyridin-3-yl-acetylamino)-hexanoic acid;

6-(2-Benzo[1,3]dioxol-5-yl-acetylamino)-2-(4′-bromo-biphenyl-4-sulfonylamino)-hexanoic acid;

2-(4′-Bromo-biphenyl-4-sulfonylamino)-6-(2-naphthalen-1-yl-acetylamino)-hexanoic acid;

2-(4′-Bromo-biphenyl-4-sulfonylamino)-6-[3-(4-chloro-phenoxy)-propionylamino]-hexanoic acid;

2-(4′-Bromo-biphenyl-4-sulfonylamino)-6-(6-phenyl-hexanoylamino)-hexanoic acid;

2-(4′-Bromo-biphenyl-4-sulfonylamino)-6-(4-thiophen-2-yl-butyrylamino)-hexanoic acid;

2-(4′-Bromo-biphenyl-4-sulfonylamino)-6-(2,4,6-triisopropylenzoylamino)-hexanoic acid;

2-(4′-Bromo-biphenyl-4-sulfonylamino)-6-isobutoxycarbonylamino-hexanoic acid;

2-(4′-Bromo-biphenyl-4-sulfonylamino)-6-(9H-fluoren-9-ylmethoxycarbonylamino)-hexanoic acid;

6-(Adamantan-1-yloxycarbonylamino)-2-(4′-bromo-biphenyl-4-sulfonylamino)-hexanoic acid; and

6-Allyloxycarbonylamino-2-(4′-bromo-biphenyl-4-sulfonylamino)-hexanoic acid.

Numerous succinamide MMP inhibitors are known and can be utilized in the method of this invention. Typical succinamides include:

2S,N¹-Dihydroxy-3R-isobutyl-N⁴-{1-([2-(2-methoxyethoxymethoxy)ethylcarbamoyl]-2,2-dimethyl-propyl }-succinamide;

2S-Allyl-N¹-hydroxy-3R-isobutyl-N⁴-{1S-[2-(2-methoxyethoxymethoxy)ethylcarbamoyl]-2-phenyl-ethyl}-succinamide;

2S-Allyl-N¹-hydroxy-3R-isobutyl-N⁴-{1S-[2-(2-methoxyethoxymethoxy)ethylcarbamoyl]-2,2-dimethyl-propyl}-succinamide;

2S-Allyl-N¹-hydroxy-3R-isobutyl-N⁴-(1S-{2-[2-(2-methoxy-ethoxy)-ethoxy]-ethylcarbamoyl]-2,2 -dimethyl-propyl}-succinamide;

2S-Allyl-N⁴-{1S-[2,2-di-(methoxymethyl)-propylcarbamoyl}-2,2-dimethyl-propyl]-N¹-hydroxy-3R-isobutyl-succinamide;

2S-Allyl-N⁴-{1S-[2,2-di-(methoxymethyl)-butylcarbamoyl]-2,2-dimethyl-propyl}-N¹-hydroxy-3R-isobutyl-succinamide;

N⁴-Hydroxy-2R-isobutyl-N^{l}-{1S-[2-(2-methoxy-ethoxy)-ethylcarbamoyl]-2,2-dimethyl-propyl}-3S-(thiophen-2-yl-sulfanylmethyl)-succinamide;

N⁴-Hydroxy-2R-isobutyl-N¹-(1S-{2-[2-(2-methoxy-ethoxy)-ethoxy]-ethylcarbamoyl )-2,2-dimethyl-propyl)-3S-(thiophen-2-yl-sulfanylmethyl)-succinamide;

N¹-{1S-[2,2-Di-(methoxymethyl)-propylcarbamoyl]-2,2-dimethyl-propyl}-N⁴hydroxy-3R-isobutyl-3S-(thiophen-2-yl-sulfanylmethyl)-succinamide;

N⁴-Hydroxy-2R-isobutyl-N¹-{1S-[2-(2-methoxy-ethoxy)-ethylcarbamoyl]-2,2-dimethyl-propyl)-3S-propyl-succinamide;

N⁴-(1S-Cyclobutylcarbamoyl-2,2dimethyl-propyl)-2S,N¹-dihydroxy-3R-isobutyl-succinamide;

N⁴-(1S-Cyclopropylcarbamoyl-2,2-dimethyl-propyl)-2S,N^{l}-dihydroxy-3R-isobutyl-succinanide;

N⁴-(1S-Cyclopentylcarbamoyl-2,2-dimethyl-propyl)-2S,N¹-dihydroxy-3R-isobutyl-succinamide;

N⁴-(1S-Cyclohexylcarbamoyl-2,2-dimethyl-propyl)-2S,N¹-dihydroxy-3R-isobutyl-succinamide;

N⁴-(1S-Cycloheptylcarbamoyl-2,2-dimethyl-propyl)-2S,N¹-dihydroxy-3R-isobutyl-succinamide;

N⁴-(1S-Cyclopropylcarbamoyl-2-mercapto-2-methyl-propyl)-2S,N¹-dihydroxy-3R-isobutyl-succinamide;

N⁴-(1S-Cyclopropylcarbamoyl-2,2-dimethyl-propyl)-2S,N¹-dihydroxy-3R-(3-phenyl-propenyl)-succinamide;

N⁴-(1S-Cyclopropylcarbamoyl-2,2-dimethyl-propyl)-2S,N¹-dihydroxy-3R-(3-phenyl-propyl)-succinamide;

N⁴-[2,2-Dimethyl-1S-(2-phenyl-cyclopropylcarbamoyl)-propyl]-2S,N¹-dihydroxy-3R-isobutyl-succinamide;

2S-Allyl-N⁴-(1-cyclopropylcarbamoyl-2,2-dimethyl-propyl)-N¹-hydroxy-3R-isobutyl-succinamide;

2S-Allyl-N⁴-(1S-cyclopropylcarbamoyl-2-mercapto-2-methyl-propyl)-N¹-hydroxy-3R-isobutyl-succinamide;

N⁴-(1S-Cyclopropylcarbamoyl-2,2-dimethyl-propyl)-N¹-hydroxy-3R-isobutyl-2S-(thiophen-2-ylsulfanylmethyl)-succinamide;

N⁴-(1S-Cyclopropylcarbamoyl-2,2-dimethyl-propyl)-N¹-hydroxy-2S-(4-hydroxy-phenylsulfanylmethyl)-3R-isobutyl-succinamide; and

N⁴-(1S-Cyclopropylcarbamoyl-2,2-dimethyl-propyl)-2S-(1,3-dioxo-1,3-dihydro-isoindol-2-ylmethyl)-N¹-hydroxy-3R-isobutyl-succinamide.

Another especially preferred group of MMP inhibitors to be utilized in combination with a statin according to the method of this invention are the sulfonated amino acid derivatives described in WO 97/27174, incorporated herein by reference. Those compounds have the general structure

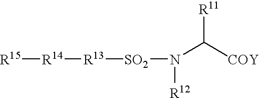

where R¹¹ is substituted or unsubstituted lower alkyl, substituted or unsubstituted aryl, substituted or unsubstituted aralkyl, substituted or unsubstituted heteroaryl, or substituted or unsubstituted heteroaryl alkyl;

R¹² is hydrogen, or a group as defined for R¹¹1;

R¹³ is a single bond, substituted or unsubstituted arylene, or substituted or unsubstituted heteroarylene;

R¹⁴ is a single bond, —(CH₂)_{1 or 2-}, —CH═CH—, —C≡C—, —CO—, —CONH—, —N═N—, NH, N-alkyl, —NHCONH—, —NHCO—, —O—, —S—, —SO₂NH—, —SO₂NH-N═CH—, or tetrazoldiyl;

R¹⁵ is substituted or unsubstituted aryl, substituted or unsubstituted heteroaryl, or substituted or unsubstituted non-aromatic heterocyclic group; and

Y is NHOH or OH.

Especially preferred compounds to be employed in the method of this invention have the above formula wherein R¹³ is phenylene or substituted phenylene. Typical of such compounds that can be employed have the formula

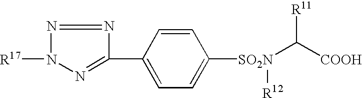

where R¹¹ and R¹² are as defined above, and R¹⁷ is substituted or unsubstituted aryl or substituted or unsubstituted heteroaryl.

Especially preferred are compounds of the formula R¹⁸

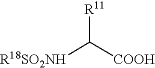

wherein R¹¹ and R¹⁸ are as follows:

| R¹¹ | R¹⁸ |
|---|---|
| 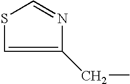 | 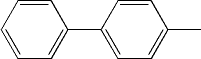 |
| 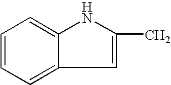 | 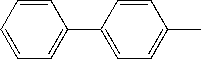 |
| 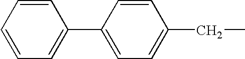 | 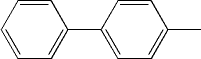 |
| CF₃CH₂— | 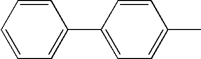 |
| 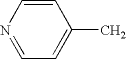 | 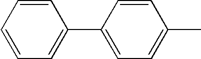 |
| HOOC—CH₂— | 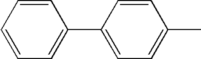 |
| 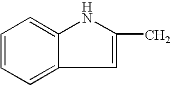 | 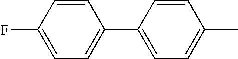 |
| (CH₂)₂CH— | 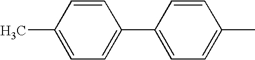 |
| (CH₃)₂CH— | 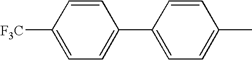 |
| 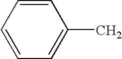 | 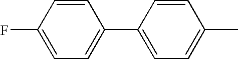 |
| (CH₃)₂CH— | 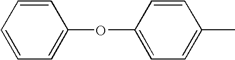 |
| 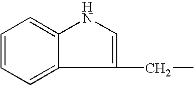 | 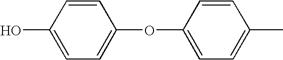 |
| 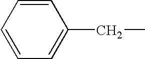 | 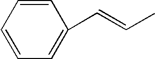 |
| 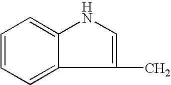 | 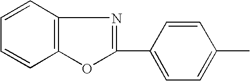 |
| 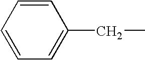 | 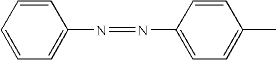 |
| (CH₃)₂CH— | 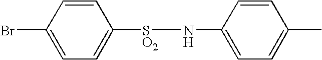 |
| (CH₃)₂CH— | 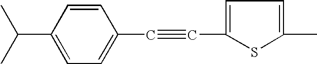 |
| 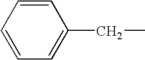 | 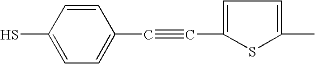 |
| (CH₃)₂CH— | 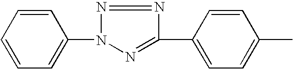 |

Especially preferred are the MMP inhibitors currently in clinical development, for example batimastat (2).

MMP compounds in clinical development include batimastat (2) for the treatment of malignant pleural effusion, and marimastat (1) for the treatment of pancreatic cancer. Galardin (3) is for the treatment of corneal ulcers, and a specific MM-1 inhibitor is RO 31-9790 -(4).

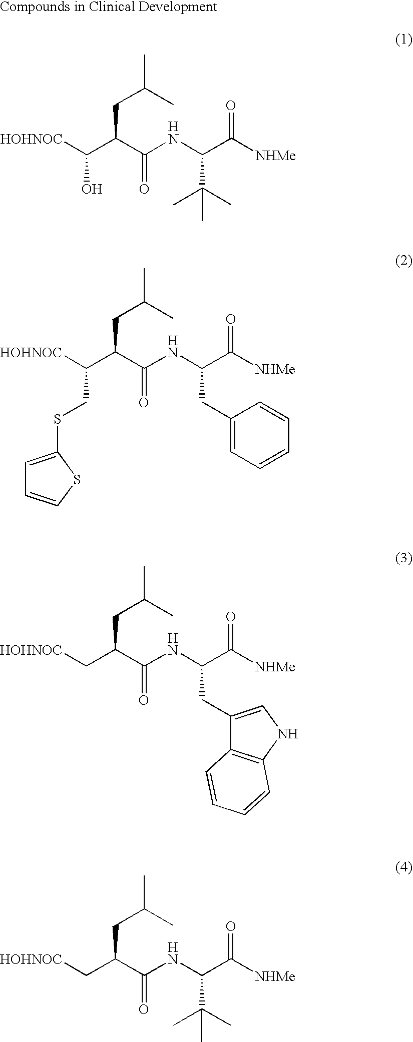

All that is required to practice the present invention is to administer to a mammal suffering from a vascular disorder, including heart failure or ventricular dilatation, or at risk of developing any such vascular disorder, an effective amount of a matrix metalloprotainase inhibitor in combination with an effective amount of a statin. Compounds which can inhibit the actions of matrix metalloproteinase enzymes can be identified utilizing routine in vitro and in vivo assays. Several compounds from within the foregoing classes have been evaluated in such standard assays and determined to be potent matrix metalloproteinase inhibitors. The assays measure the amount by which a test compound reduces the hydrolysis of a thiopeptolide substrate caused by a matrix metalloproteinase enzyme. Such assays are described in detail by Ye, et al., in Biochemistry, Vol. 31, No 45, 1992, (11231-11235), which is incorporated herein by reference.

Thiopeptolide substrates show virtually no decomposition or hydrolysis in the absence of a matrix metalloproteinase enzyme. A typical thiopeptolide substrate commonly utilized for assays is Ac-Pro-Leu-Gly-thioester-Leu-Leu-Gly-OEt. A 100 μl assay mixture will contain 50 mM of 2-morpholinoethane sulfonic acid monohydrate (MES, pH 6.0) 10 mM CaCl₂, 100 μM thiopeptolide substrate, and 1 mM 5,5′-dithio-bis-(2-nitro-benzoic acid) (DTNB). The thiopeptolide substrate concentration is varied from 10 to 800 μM to obtain Km and Kcat values. The change in absorbance at 405 nm is monitored on a Thermo Max microplate reader (moleucular Devices, Menlo Park, Calif.) at room temperature (22° C.). The calculation of the amount of hydrolysis of the thiopeptolide substrate is based on E₄₁₂=13600 m⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Several representative compounds have been evaluated for their ability to inhibit various matrix metalloproteinase enzymes. Table I below presents inhibitory activity for compounds from various classes. In the table, MMP-1 refers to interstitial collagenase; MMP-2 refers to Gelatinase A; MMP-3 refers to stromelysin; MMP-7 refers to matrilysin; and MB-9 refers to Gelatinase B. Test compounds were evaluated at various concentrations in order to determine their respective IC₅₀ values, the micromolar concentration of compound required to cause a 50% inhibition of the hydrolytic activity of the respective enzyme.

TABLE I
(IC₅₀ μM)

| | MMP1 | MMP2 | MMP3 | MMP7 | MMP9 |
|---|---|---|---|---|---|
| Batimastat is N⁴⁻Hydroxy-N¹⁻[2-(methylamine)-2-oxo--1- | 0.005 | 0.004 | 0.02 | | |
| (phenylmethyl)ethyl]-2-(2-methylpropyl)-3-[(2-thienylthio)methyl]- | | | | | |
| butanediamide | | | | | |
| CDP-845 (Celltech) | 0.303 | 0.0015 | 0.01 | | |
| CGS 27023A (Ciba-Giegy) | 0.033 | 0.01 | 0.01 | | 0.008 |
| Galardin is N⁴⁻Hydroxy-N¹⁻[2-(methylamine)-2-oxo-1-(3- | 0.0004 | 0.0005 | 27 | | 0.0002 |
| indolylmethyl)ethyl]-2-(2-methylpropyl)-butanediamide | | | | | |
| U24522 (Merck) | | 0.05 | 0.02 | | |
| RO-31-9790 (Roche) | 0.0055 | 0.006 | 0.47 | | |
| 4-Oxo-4-[4-(4-phenyl-piperidin-1-yl)-phenyl]-butyric acid | | 1.3 | 0.14 | | |
| N-Hydroxy-4-oxo-4-[4-(4-phenyl-piperidin-1-yl)-phenyl]-butyramide | | 0.04 | 0.02 | | |
| 4-Oxo-4-[4-(4-phenyl-piperazin-1-yl)-phenyl]-butyric acid | | 1.6 | 0.25 | | |
| [4-(4-Phenyl-piperidin-1-yl)-benzenesulfonylamino]-acetic acid | | 0.21 | 0.02 | | |
| N-Hydroxy-2-[4-(4-phenyl-piperidin-1-yl)-benzene-sulfonylamino]-acetamide | | 0.81 | 0.019 | | |
| (S)-3-Phenyl-2-[4-(4-phenyl-piperidin-1-yl)-benzene-sulfonylamino]- | | 0.22 | 0.014 | | |
| propionic acid | | | | | |
| (S)-2-[4-(4-Benzyl-piperidin-1-yl)-benzenesulfonyl-amino]-3-phenyl- | | 0.088 | 0.021 | | |
| propionic acid | | | | | |
| (S)-2-{4-[-4-(4-Methoxy-phenyl)-piperazin-1-yl]-benzenesulfonylamino}-3- | | 0.033 | 0.014 | | |
| phenyl-propionic acid | | | | | |
| (S)-2-(4′-Bromo-biphenyl-4-sulfonylamino)-3-methyl-butyric acid | 3.24 | 0.025 | 0.012 | | |
| (S)-3-Methyl-2-(4′-nitro-biphenyl-4-sulfonylamino)-butyric acid; | | 0.013 | 0.10 | | |
| (S)-2-(4′-Amino-biphenyl-4-sulfonylamino)-3-methyl-butyric acid | | 0.044 | 0.067 | | |
| (S)-2-(4′-Bromo-biphenyl-4-sulfonylamino)-3-phenyl-propionic acid | | 0.026 | 0.026 | | |
| 4-(4′-Chloro-biphenyl-4-yl)-4-hydroxyimino-butyric acid | | 0.39 | 0.12 | | |
| 4-(4′-Bromo-biphenyl-4-yl)-4-hydroxyimino-butyric acid | | 0.058 | 0.11 | | |
| 4-(4′-Chloro-biphenyl-4-yl)-4-(dimethylhydrazono)-butyric acid | | 0.73 | 0.93 | | |
| (±)-4-(4′-Chloro-biphenyl-4-yl)-4-hydroxy-butyric acid | | 0.15 | 0.28 | | |
| (S)-2-(Dibenzofuran-2-sulfonylamino)-4-phenyl-butyric acid | | 0.265 | 0.46 | | |
| (L)-2-(Dibenzofuran-2-sulfonylamino)-4-methyl-pentanoic acid | | 0.32 | 1.18 | | |
| (L)-2-(Dibenzofuran-2-sulfonylamino)-3-phenyl-propionic acid | | 0.89 | 0.72 | | |
| (L)-2-(Dibenzofuran-2-sulfonylamino)-3-methyl-butyric acid | | 0.084 | 0.23 | | |
| (L)-2-(Dibenzofuran-2-sulfonylamino)-3-tritylsulfanyl-propionic acid | | 9.4 | 14.4 | | |
| (L)-2-(Dibenzofuran-2-sulfonylamino)-3-mercapto-propionic acid | | 4.45 | 0.69 | | |
| (S)-4-Dibenzofuran-2-yl-4-oxo-2-(2,2,2-trifluoroacetylamino)-butyric acid | | 0.72 | 1.33 | | |
| (S)-2-Amino-4-dibenzofuran-2-yl-4-oxo-butyric acid | | 3.8 | 33.0 | | |
| (S)-2-Acetylamino-4-dibenzofuran-2-yl-4-oxo-butyric acid | | 0.16 | 1.55 | | |
| (S)-4-Dibenzofuran-2-yl-4-oxo-2-phenylacetylamino-butyric acid | | 0.084 | 0.33 | | |
| (S)-4-Dibenzofuran-2-yl-4-oxo-2-(3-phenyl-propionylamino)-butyric acid | | 0.096 | 0.28 | | |

The pharmaceutical combinations and methods of this invention are all adapted to therapeutic use as agents in the prevention and treatment of atherosclerosis, angina pectoris, and a condition characterized by the presence of both hypertension and hyperlipidemia in mammals, particularly humans. Further, since these diseases and conditions are closely related to the development of cardiac disease and adverse cardiac conditions, these combinations and methods, by virtue of their action as antiatherosclerotics, antianginals, antihypertensives, and antihyperlipidemics, are useful in the management of cardiac risk in subjects at risk of developing adverse cardiac conditions and in subjects at risk of suffering adverse cardiac events and heart failure such as CoF and ventricular dilatation.

The utility of the compositions of the present invention as medical agents in the treatment of vascular disorders such as atherosclerosis and CHF in mammals (e.g., humans) is demonstrated by the activity of the compounds of this invention in conventional assays and in a clinical protocol such as that described below. The following examples are illustrative only, and are not intended to limit the invention in any way.

### EXAMPLE 1

The following animal assay established the ability of an MMP inhibitor together with a statin to treat vascular disorders.

Four groups of male New Zealand White rabbits were fed a chow diet (Ralston Purina) combining 2% cholesterol, 3% peanut oil, 3% coconut oil diet for 1 week prior to administering test compounds, and throughout the test. Water was available ad libitum. One group of 10 animals served as untreated controls. Ten milligrams per kilograms of PD166793 (CI-1026) was administered daily to one group of 10 animals for 8 weeks. Five milligrams per kilogram atorvastatin calcium was administered daily to a group of 10 animals for 8 weeks. The combination of both agents was administered at the same doses to a group of 10 animals for 8 weeks. At necropsy, blood was collected for plasma cholesterol measurements, aortic arch was removed for morphologic, morphometric and biochemical analysis. The gross surface extent of atherosclerosis within the aortic arch, lesion cross-sectional area and monocyte-macrophage content were measured as indices of antiatherosclerotic activity.

Biochemical Methods. Plasma total cholesterol and triglyceride levels were measured enzymatically throughout the study on an Abbott VP Series II Bichromatic Analyzer (Chicago, Ill.), using the Boehringer-Mannheim total cholesterol reagent (Indianapolis, Ind.) and the Abbott triglyceride reagent (Chicago, Ill.). The lipid measurements were made monthly or biweekly throughout the study on plasma samples collected 24 hours post-meal. The aortic arch was assayed for its cholesteryl ester (CE), free cholesterol, and total phospholipid content. The lipids were extracted in chloroform: methanol (2:1 v/v) and 300-500 μL of an internal standard, i.e., 200 mg/mL of a solution of 4-hydroxy-cholesterol in ethyl acetate: acetone (2:1 v/v), was added to the extracts of the aortic samples. After extraction, the organic phase was dried under nitrogen and redissolved in isooctane/tetrahydrofuran (97:3 v/v). The lipid content and composition of the aortic arch were measured using an HPLC method.

Cytochemical Methods. For histologic evaluation of the aortic arch lesions and for quantification of aortic arch cross-sectional lesion area, a 1 cm segment of the ascending aorta distal to the aortic valves was fixed in 10% neutral buffered formalin for 24 hours. The vessels were dehydrated, cleared in xylene, and infiltrated with molten paraffin (<60EC) using a Tissue Tek VIP autoprocessor (Miles Scientific, Elkhart, Ind.). The tissue segments were embedded in paraffin and sectioned at 5 urn with a Reichert-Jung microtome (Baxter, McGraw Park, Ill.). In order to obtain a thorough representation of the histologic appearance of the aortic arch lesions, 3 ribbons of 20 sections each were cut. Each ribbon of sections was spaced approximately 100 μm apart. Three pairs of sections, i.e., 1 pair from each ribbon, were affixed to cleaned 3-aminopropyltriethoxy-silane coated glass slides and stored until stained. The general histologic character was evaluated in Verhoeff's elastica stained sections.

Morphometric Methods. Gross extent of atherosclerosis within the aortic arch was measured. The area of the aortic arch distal to the 1 cm segment taken for histologic evaluation to the first intercostal ostia was removed from the animal, opened longitudinally, and images of the surface of the vessel were collected using a digital camera. The lesions were identified as raised, opaque areas, and their area was deternined using the Image Pro Plus image analysis software. The area of the entire aortic arch was also determined. The percentage of aortic arch covered by atherosclerotic lesions was calculated.

The cross-sectional area and macrophage content of lesions located distal to the aortic valve ring were also measured. Sections of the aortic arch, a site of hypercholesterolemia-induced lesions, stained using the Verhoeff's elastica procedure were used for quantification of lesion cross-sectional area. The internal elastic lamina (IEL) was identified as a blue-black ring and images of that region were collected using a digital camera. The area within the IEL was quantified using the Image Pro Plus image analysis software. The area of the lumen of the aortic arch was also quantified in a similar fashion. Lesion area was defined as the difference between the area circumscribed by the internal elastic lamina and the lumen area. Contiguous sections stained for monocyte-macrophages using antibodies specific for rabbit macrophages were used for quantification of macrophage area. The red- or brown-stained regions within the aortic arch lesions were identified, and images were collected using a digital camera. Since the macrophages were darker than the surrounding lesion area, a histogram of grey levels was used to identify an inflection point in the image grey level intensities, which arbitrarily delineated the grey levels associated with macrophages. After delineation of the macrophages, their area was quantified using the Image Pro Plus image analysis software.

The data thus generated established that MMP inhibitor-statin combinations are surprisingly useful for treating vascular disorders.

### EXAMPLE 2

### Effect of MMP Inhibitor and a Statin, Alone and in Combination, on the Treatment of Atherosclerosis

This study is a prospective randomized evaluation of the effect of a combination of an MMP inhibitor or a pharmaceutically acceptable salt thereof and a statin on the progression/regression of coronary and carotid artery disease. The study is used to show that a combination of an MMP inhibitor, e.g., 2-(4′-bromophenyl-4-sulfonylamino)-3-methyl-butyric acid, or a pharmaceutically acceptable acid addition salt, and a statin, e.g. atorvastatin calcium, is effective in slowing or arresting the progression or causing regression of existing coronary artery disease (CAD) as evidenced by changes in coronary angiography or carotid ultrasound, in subjects with established disease.

This study is an angiographic documentation of coronary artery disease carried out as a double blind, placebo-controlled trial of a minimum of about 500 subjects and preferably of about 780 to about 1200 subjects. It is especially preferred to study about 1200 subjects in this study. Subjects are admitted into the study after satisfying certain entry criteria set forth below.

Entry criteria: Subjects accepted for entry into this trial must satisfy certain criteria. Thus, the subject must be an adult, either male or female, aged 18 to 80 years of age in whom coronary angiography is clinically indicated. Subjects will have angiographic presence of a significant focal lesion such as 30% to 50% on subsequent evaluation by quantitative coronary angiography (QCA) in a minimum of one segment (non-PTCA, non-bypassed, or non-MI vessel) that is judged not likely to require intervention over the next 3 years. It is required that the segments undergoing analysis have not been interfered with. Since percutaneous transluminal cardiac angioplasty (PTCA) interferes with segments by the insertion of a balloon catheter, non-PTCA segments are required for analysis. It is also required that the segments to be analyzed have not suffered a thrombotic event, such as a myocardial infarct (MI). Thus, the requirement for non-MI vessels. Segments that will be analyzed include: left main, proximal, mid and distal left anterior descending, first and second diagonal branch, proximal and distal left circumflex, first or largest space obtuse marginal, proximal, mid and distal right coronary artery. Subjects will have an ejection fraction of greater than 30% determined by catheterization or radionuclide ventriculography or ECHO cardiogram at the time of the qualifying angiogram or within the previous 3 months of the acceptance of the qualifying angiogram provided no intervening event such as a thrombotic event or procedure such as PTCA has occurred.

Generally, due to the number of patients and the physical limitations of any one facility, the study is carried out at multiple sites. At entry into the study, subjects undergo quantitative coronary angiography as well as B-mode carotid artery ultrasonography and assessment of carotid arterial compliance at designated testing centers. This establishes baselines for each subject. Once admitted into the test, subjects are randomized to receive the MMP inhibitor (200 mg) and placebo or a statin (dose is dependent upon the particular statin used; however, generally 80 mg will be used at first) and placebo or MMP inhibitor (200 mg) and a statin (80 mg). It will be recognized by a skilled person that the free base form or other salt forms of MMP inhibitors or the free base form or other salt forms of the statin may be used in this invention. Calculation of the dosage amount for these other forms of the statin and MMP inhibitor is easily accomplished by performing a simple ratio relative to the molecular weights of the species involved. The amount of MMP inhibitor may be varied as required. Generally, a subject will start out taking 200 mg, and the amount will be titrated down to as little as 50 mg as determined by the clinical physician. The amount of the statin will similarly be titrated down from 80 mg if it is determined by the physician to be in the best interests of the subject. The subjects are monitored for a 1- to 3-year period, generally 3 years being preferred. B-mode carotid ultrasound assessment of carotid artery atherosclerosis and compliance are performed at regular intervals throughout the study.

Generally, 6-month intervals are suitable. Typically this assessment is performed using B-mode ultrasound equipment. However, a person skilled in the art may use other methods of performing this assessment. Coronary angiography is performed at the conclusion of the 1- to 3-year treatment period. The baseline and posttreatment angiograms and the intervening carotid artery B-mode ultrasonograms are evaluated for new lesions or progression of existing atherosclerotic lesions. Arterial compliance measurements are assessed for changes from baseline and over the 6-month evaluation periods.

The primary objective of this study is to show that the combination of MMP inhibitor or a pharmaceutically acceptable acid addition salt and a statin reduces the progression of atherosclerotic lesions as measured by quantitative coronary angiography (QCA) in subjects with clinical coronary artery disease. QCA measures the opening in the lumen of the arteries measured.

The primary endpoint of the study is the change in the average mean segment diameter of the coronary artery tree. Thus, the diameter of an arterial segment is measured at various portions along the length of that segment. The average diameter of that segment is then determined. After the average segment diameter of many segments has been determined, the average of all segment averages is determined to arrive at the average mean segment diameter. The mean segment diameter of subjects taking a statin and MMP inhibitor or a pharmaceutically acceptable acid addition salt will decline more slowly, will be halted completely, or there will be an increase in the mean segment diameter. These results represent slowed progression of atherosclerosis, no change in the progression of atherosclerosis, and regression of atherosclerosis, respectively.

The secondary objective of this study is that the combination of MMP inhibitor or a pharmaceutically acceptable acid addition salt and a statin reduces the rate of progression of atherosclerosis in the carotid arteries as measured by the slope of the maximum intimal-medial thickness measurements averaged over 12 separate wall segments (Mean Max) as a function of time, more than does the MMP inhibitor or a pharmaceutically acceptable acid addition salt or a statin alone. The intimal-medial thickness of subjects taking a statin and an MMP inhibitor or a pharmaceutically acceptable salt thereof will increase more slowly, will cease to increase, or will decrease. These results represent slowed progression of atherosclerosis, halted progression of atherosclerosis, and regression of atherosclerosis, respectively. Further, these results may be used to facilitate dosage determinations.

The utility of the compounds of the present invention as medical agents in the treatment of angina pectoris in mammals (e.g., humans) is demonstrated by the activity of the compounds of this invention in conventional assays and the clinical protocol described below.

### EXAMPLE 3

### Effect of MMP Inhibitor and a Statin, Alone and in Combination, on the Treatment of Angina

This study is a double blind, parallel-arm, randomized study to show the effectiveness of MMP inhibitor or a pharmaceutically acceptable acid addition salt thereof and a statin given in combination in the treatment of symptomatic angina.

Entry criteria: Subjects are males or females between 18 and 80 years of age with a history of typical chest pain associated with one of the following objective evidences of cardiac ischemia: (1) stress test segment elevation of about one millimeter or more from the ECG; (2) positive treadmill stress test; (3) new wall motion abnormality on ultrasound; or (4) coronary angiogram with a significant qualifying stenosis. Generally a stenosis of about 30% to 50% is considered to be significant.

Each subject is evaluated for about 10 to 32 weeks. At least 10 weeks are generally required to complete the study. Sufficient subjects are used in this screen to ensure that about 200 to 800 subjects and preferably about 400 subjects are evaluated to complete the study. Subjects are screened for compliance with the entry criteria, set forth below, during a 4-week run-in phase. After the screening criteria are met, subjects are washed out from their current anti-anginal medication and stabilized on a long acting nitrate such as nitroglycerine, isosorbide-5-mononitrate or isosorbide dinitrate. The term “washed out”, when used in connection with this screen, means the withdrawal of current anti-anginal medication so that substantially all of said medication is eliminated from the body of the subject. A period of 8 weeks is preferably allowed for both the washout period and for the establishment of the subject on stable doses of said nitrate. Subjects having one or two attacks of angina per week while on stable doses of long acting nitrate are generally permitted to skip the washout phase. After subjects are stabilized on nitrates, the subjects enter the randomization phase provided the subjects continue to have either one or two angina attacks per week. In the randomization phase, the subjects are randomly placed into one of the four arms of the study set forth below. After completing the washout phase, subjects in compliance with the entry criteria undergo 24-hour ambulatory electrocardiogram (ECG) such as Holter monitoring, exercise stress testing such as a treadmill, and evaluation of myocardial perfusion using photon emission tomography (PET) scanning to establish a baseline for each subject. When conducting a stress test, the speed of the treadmill and the gradient of the treadmill can be controlled by a technician. The speed of the treadmill and the angle of the gradient are generally increased during the test. The time intervals between each speed and gradient increase is generally determined using a modified Bruce Protocol.

After the baseline investigations have been completed, subjects are initiated on one of the following four arms of the study: (1) placebo; (2) a statin (about 2.5 mg to about 160 mg); (3) MMP inhibitor (about 25 mg to about 200 mg); or (4) a combination of the above doses of MMP inhibitor and a statin together. The subjects are then monitored for 2 to 24 weeks. It will be recognized by a skilled person that the free base form or other salt forms of MMP inhibitor or the free base form or other salt forms of the statin may be used in this invention. Calculation of the dosage amount for these other forms of the statin and MMP inhibitor is easily accomplished by performing a simple ratio relative to the molecular weights of the species involved.

After the monitoring period has ended, subjects will undergo the following investigations: (1) 24-hour ambulatory ECG, such as Holter monitoring; (2) exercise stress testing (e.g., treadmill using said modified Bruce Protocol); and (3) evaluation of myocardial perfusion using PET scanning. Patients keep a diary of painful ischemic events and nitroglycerine consumption. It is generally desirable to have an accurate record of the number of anginal attacks suffered by the patient during the duration of the test. Since a patient generally takes nitroglycerin to ease the pain of an anginal attack, the number of times that the patient administers nitroglycerine provides a reasonably accurate record of the number of anginal attacks.

To demonstrate the effectiveness and dosage of the drug combination of this invention, the person conducting the test will evaluate the subject using the tests described. Successful treatment will yield fewer instances of ischemic events as detected by ECG, will allow the subject to exercise longer or at a higher intensity level on the treadmill or to exercise without pain on the treadmill, or will yield better perfusion or fewer perfusion defects on PET.

The utility of the compounds of the present invention as medical agents in the treatment of hypertension and hyperlipidemia in mammals (e.g., humans) suffering from a combination of hypertension and hyperlipidemia is demonstrated by the activity of the compounds of this invention in conventional assays and the clinical protocol described below.

### EXAMPLE 4

### Effects of MMP Inhibitor and a Statin, Alone and in Combination, on the Treatment of Subjects Having Both Hypertension and Hyperlipidemia

This study is a double blind, parallel-arm, randomized study to show the effectiveness of an MMP inhibitor or a pharmaceutically acceptable acid addition salt thereof and a statin given in combination in controlling both hypertension and hyperlipidemia in subjects who have mild, moderate, or severe hypertension and hyperlipidemia.

Each subject is evaluated for 10 to 20 weeks and preferably for 14 weeks. Sufficient subjects are used in this screen to ensure that about 400 to 800 subjects are evaluated to complete the study.

Entry criteria: Subjects are male or female adults between 18 and 80 years of age having both hyperlipidemia and hypertension. The presence of hyperlipidemia is evidenced by evaluation of the LDL cholesterol level of the subject relative to certain positive risk factors. If the subject has no coronary heart disease (CHD) and has less than two positive risk factors, then the subject is considered to have hyperlipidemia, which requires drug therapy if the LDL of the subject is ≧190 mg/dL. If the subject has no CHD and has two or more positive risk factors, then the subject is considered to have hyperlipidemia, which requires drug therapy if the LDL of the subject is ≧160 mg/dL. If the subject has CHD, then the subject is considered to have hyperlipidemia if the LDL of the subject is ≧130 mg/dL.

Positive risk factors include: (1) male over 45, (2) female over 55 wherein said female is not undergoing hormone replacement therapy (HRT), (3) family history of premature cardiovascular disease, (4) the subject is a current smoker, (5) the subject has diabetes, (6) an HDL of less than 45 mg/d and (7) the subject has hypertension. An HDL of >60 mg/dL is considered a negative risk factor and will offset one of the above-mentioned positive risk factors.

The presence of hypertension is evidenced by a sitting diastolic blood pressure (BP) of >90 mmHg or sitting systolic BP of >140 mmHg. All blood pressures are generally determined as the average of Free measurements taken 5 minutes apart.

Subjects are screened for compliance with the entry criteria set forth above. After all screening criteria are met, subjects are washed out from their current antihypertensive and lipid lowering medication and are placed on the NCEP ATP II Step 1 diet The NCEP ATP II (adult treatment panel, 2nd revision) Step 1 diet sets forth the amount of saturated and unsaturated fat which can be consumed as a proportion of the total caloric intake. The term “washed out”, where used in connection with this screen, means the withdrawal of current antihypertensive and lipid-lowering medication so that substantially all of said medication is eliminated from the body of the subject. Newly diagnosed subjects generally remain untreated until the test begins. These subjects are also placed on the NCEP Step 1 diet. After the 4week washout and diet stabilization period, subjects undergo the following baseline investigations: (1) blood pressure and (2) fasting lipid screen. The fasting lipid screen determines baseline lipid levels in the fasting state of a subject. Generally, the subject abstains from food for 12 hours, at which time lipid levels are measured.

After the baseline investigations are performed, subjects are started on one of the following: (1) a fixed dose of MMP inhibitor, generally about 25 to 200 mg; (2) a fixed dose of a statin, generally about 2.5 mg to about 160 mg; or (3) a combination of the above doses of MMP inhibitor and a statin together. It will be recognized by a skilled person that the free base form or other salt forms of MMP inhibitor or the free base form or other salt forms of the statin may be used in this invention. Calculation of the dosage amount for these other forms of the statin and MMP inhibitor is easily accomplished by performing a simple ratio relative to the molecular weights of the species involved. Subjects remain on these doses for a minimum of 6 weeks, and generally for no more than 8 weeks. The subjects return to the testing center at the conclusion of the 6 to 8 weeks so that the baseline evaluations can be repeated. The blood pressure of the subject at the conclusion of the study is compared with the blood pressure of the subject upon entry. The lipid screen measures the total cholesterol, LDL-cholesterol, HDL-cholesterol, triglycerides, apoB, very low-density lipoprotein (VLDL) and other components of the lipid profile of the subject. Improvements in the values obtained after treatment relative to pretreatment values indicate the utility of the drug combination.

The utility of the compounds of the present invention as medical agents in the management of cardiac risk in mammals (e.g., humans) at risk for an adverse cardiac event is demonstrated by the activity of the compounds of this invention in conventional assays and the clinical protocol described below.

### EXAMPLE 5

### Effects of an MMP Inhibitor and a Statin, Alone and in Combination, on Subjects at Risk of Future Cardiovascular Events

This study is a double-blind, parallel-arm, randomized study to demonstrate the effectiveness of carboxyalkylether or a pharmaceutically acceptable acid addition salt and a statin given in combination in reducing the overall calculated risk of future events in subjects who are at risk for having future cardiovascular events. This risk is calculated by using the Framingham Risk Equation. A subject is considered to be at risk of having a future cardiovascular event if that subject is more than one standard deviation above the mean as calculated by the Framingham Risk Equation. The study is used to evaluate the efficacy of a fixed combination of carboxylalkylether or a pharmaceutically acceptable acid addition salt and a statin in controlling cardiovascular risk by controlling both hypertension and hyperlipidemia in patients who have both mild to moderate hypertension and hyperlipidemia.

Each subject is evaluated for 10 to 20 weeks and preferably for 14 weeks. Sufficient subjects are recruited to ensure that about 400 to 800 subjects are evaluated to complete the study.

Entry criteria: Subjects included in the study are male or female adult subjects between 18 and 80 years of age with a baseline 5-year risk, which risk is above the median for said subject's age and sex, as defined by the Framingham Heart Study, which is an ongoing prospective study of adult men and women showing that certain risk factors can be used to predict the development of coronary heart disease. The age, sex, systolic and diastolic blood pressure, smoking habit, presence or absence of carbohydrate intolerance, presence or absence of left ventricular hypertrophy, serum cholesterol, and HDL of more than one standard deviation above the norm for the Framingham Population are all evaluated in determining whether a patient is at risk for adverse cardiac event. The values for the risk factors are inserted into the Framingham Risk Equation and calculated to determine whether a subject is at risk for a future cardiovascular event.

Subjects are screened for compliance with the entry criteria set forth above. After all screening criteria are met, patients are washed out from their current antihypertensive and lipid lowering medication and any other medication which will impact the results of the screen. The patients are then placed on the NCEP ATP II Step 1 diet, as described above. Newly diagnosed subjects generally remain untreated until the test begins. These subjects are also placed on the NCEP ATP II Step 1 diet. After the 4week washout and diet stabilization period, subjects undergo the following baseline investigations: (1) blood pressure; (2) fasting; (3) lipid screen; (4) glucose tolerance test; (5) ECG; and (6) cardiac ultrasound. These tests are carried out using standard procedures well known to persons skilled in the art. The ECG and the cardiac ultrasound are generally used to measure the presence or absence of left ventricular hypertrophy.

After the baseline investigations are performed, patients will be started on one of the following: (1) a fixed dose of MMP inhibitor (about 25 to 200 mg); (2) a fixed dose of a statin (about 2.5 mg to about 160 mg); or (3) the combination of the above doses of MMP inhibitor and a statin. Patients are kept on these doses and are asked to return in 6 to 8 weeks so that the baseline evaluations can be repeated. At this time, the new values are entered into the Framingham Risk Equation to determine whether the subject has a lower, greater, or no change in the risk of future cardiovascular event.

The above assays demonstrating the effectiveness of an MMP inhibitor or pharmaceutically acceptable acid addition salts thereof and a statin such as atorvastatin or pharmaceutically acceptable salts thereof in the treatment of angina pectoris, atherosclerosis, hypertension and hyperlipidemia together, and the management of cardiac risk, also provide a means whereby the activities of the compounds of this invention can be compared between themselves and with the activities of other known compounds. The results of these comparisons are useful for determining dosage levels in mammals, including humans, for the treatment of such diseases.

The following dosage amounts and other dosage amounts set forth elsewhere in this specification and in the appendant claims are for an average human subject having a weight of about 65 kg to about 70 g. The skilled practitioner will readily be able to determine the dosage amount required for a subject whose weight falls outside the 65 to 70 kg range, based upon the medical history of the subject and the presence of diseases, e.g., diabetes, in the subject. All doses set forth herein, and in the appendant claims, are daily doses.

In general, in accordance with this invention, the MMP inhibitor is generally administered in a dosage of about 25 mg to about 500 mg. Preferably, MMP inhibitor is administered in a dosage of about 5 mg to about 100 mg. It will be recognized by a skilled person that the free base form or other salt forms of MMP inhibitor may be used in this invention. Calculation of the dosage amount for these other forms of or the free base form or other salt forms of MMP inhibitor is easily accomplished by performing a simple ratio relative to the molecular weights of the species involved.

In general, in accordance with this invention, the above statins are administered in the following dosage amounts:

Simvastatin, generally about 2.5 mg to about 160 mg and preferably about 10 mg to about 40 mg;

Pravastatin, generally about 2.5 mg to about 160 mg and preferably about 10 mg to about 40 mg;

Cerivastatin, generally about 25 μg to about 5 mg and preferably about 1 mg to about 3.2 mg;

Fluvastatin, generally about 2.5 mg to about 160 mg and preferably about 20 mg to about 80 mg;

Lovastatin, generally about 2.5 mg to about 160 mg and preferably about 10 mg to about 80 mg; and

Atorvastatin, generally about 2.5 mg to about 160 mg and preferably about 10 mg to about 80 mg.

It will be recognized by a skilled person that the free base form or other salt forms of the above statins may be used in this invention. Calculation of the dosage amount for these other forms of or the free base form or other salt forms said statins is easily accomplished by performing a simple ratio relative to the molecular weights of the species involved.

The compounds of the present invention are generally administered in the form of a pharmaceutical composition comprising at least one of the compounds of this invention together with a pharmaceutically acceptable carrier or diluent. Thus, the MMP inhibitors and the statins can be administered either individually or together in any conventional oral, parenteral, or transdermal dosage form.

For oral administration, a pharmaceutical composition can take the form of solutions, suspensions, tablets, pills, capsules, powders, and the like. Tablets containing various excipients such as sodium citrate, calcium carbonate, and calcium phosphate are employed along with various disintegrants such as starch and preferably potato or tapioca starch and certain complex silicates, together with binding agents such as polyvinylpyrrolidone, sucrose, gelatin, and acacia. Additionally, lubricating agents such as magnesium stearate, sodium lauryl sulfate, and talc are often very useful for tableting purposes. Solid compositions of a similar type are also employed as fillers in soft- and hard-filled gelatin capsules; preferred materials in this connection also include lactose or milk sugar, as well as high molecular weight polyethylene glycols. When aqueous suspensions and/or elixirs are desired for oral administration, the compounds of this invention can be combined with various sweetening agents, flavoring agents, coloring agents, emulsifying agents, and/or suspending agents, as well as such diluents as water, ethanol, propylene glycol, glycerin, and various like combinations thereof.

The combinations of this invention may also be administered in a controlled release formulation such as a slow release or a fast release formulation. Such controlled release formulations of the combination of this invention may be prepared using methods well known to those skilled in the art. The method of administration will be determined by the attendant physician or other person skilled in the art after an evaluation of the subject's condition and requirements. The generally preferred formulation of atorvastatin calcium is Lipitor® as described in U.S. Pat. No. 5,686,104 incorporated herein by reference.

For purposes of parenteral administration solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous, and intraperitoneal injection purposes. In this connection, the sterile aqueous media employed are all readily obtainable by standard techniques well known to those skilled in the art.

Methods of preparing various pharmaceutical compositions with a certain amount of active ingredient are known, or will be apparent in light of this disclosure, to those skilled in this art. For examples, see Remington's Pharmcaceutical Sciences, Mack Publishing Company, Easter, Pennsylvania., 15th Edition (1975).

Pharmaceutical compositions according to the invention may contain 0.1% to 95% of the compound(s) of this invention, preferably 1% to 70%. In any event, the composition or formulation to be administered will contain a quantity of a compound(s) according to the invention in an amount effective to prevent or treat the condition or disease of the subject being treated, namely a vascular disorder, including CHF.

Since the present invention relates to the treatment of diseases and conditions with a combination of active ingredients, which may be administered separately, the invention also relates to combining separate pharmaceutical compositions in kit form. The kit includes two separate pharmaceutical compositions: an MMP inhibitor or a pharmaceutically acceptable acid addition salt thereof, and a statin or a pharmaceutically acceptable salt thereof. The kit includes container means for containing the separate compositions such as a divided bottle or a divided foil packet; however, the separate compositions may also be contained within a single, undivided container. Typically, the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

It should be understood that the invention is not limited to the particular embodiments described herein, but that various changes and modifications may be made without departing from the spirit and scope of this novel concept as defined by the following claims.

1. (Cancelled).

2. (Cancelled).

3. (Cancelled).

4. A pharmaceutical composition of claim 1 comprising:
a. an amount of an MMP-1, MMP-2, or MMP-3 inhibitor or a pharmaceutically acceptable acid addition salt thereof having the formula 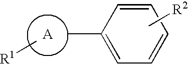
wherein:
A is phenyl or 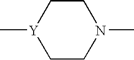
where Y is CH or N;
R¹ is a substituent such as alkyl, aryl, halo, amino, substituted and disubstituted amino, and alkoxy;
R² is carboxyalkyl ketone or 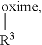
or a -SO₂NHCHCOOH
where R³ is alkyl, substituted alkyl, amino, substituted and disubstituted amino, and aryl;
b. an amount of atorvastatin or a pharmaceutically acceptable salt thereof; and
C. a pharmaceutically acceptable carrier or diluent.

5. (Cancelled).

6. (Cancelled).

7. (Cancelled).

8. (Cancelled).

9. (Cancelled).

10. (Cancelled).

11. (Cancelled).

12. (Cancelled).

13. (Cancelled).

14. (Cancelled).

15. (Cancelled).

16. (Cancelled).

17. (Cancelled).

18. (Cancelled).

19. (Cancelled).

20. (Cancelled).

21. (Cancelled).

22. (Cancelled).

23. (Cancelled).

24. (Cancelled).

25. (Cancelled).

26. (Cancelled).

27. (Cancelled).

28. (Cancelled).

29. The pharmaceutical composition of claim 4 wherein in the MMP-1, MMP-2, or MMP-3 inhibitor of formula I is 2-(4′-bromobiphenyl-4-sulfonylamino)-3-methylbutyric acid.
